(19) United States
(12) Patent Application Publication
(10) Pub. No.: US 2025/0090700 A1
(43) Pub. Date: Mar. 20, 2025

(54) HUMANIZED ANTIBODY THAT BINDS TO HEG1 PROTEIN AND COMPLEX OF ANTIBODY AND RADIONUCLIDE

(71) Applicants: NIHON MEDI-PHYSICS CO., LTD., Tokyo (JP); Kanagawa Prefectural Hospital Organization, Yokohama-shi (JP)

(72) Inventors: Takuya TAKEDA, Tokyo (JP); Minoru KAWATANI, Tokyo (JP); Gota TONOYA, Tokyo (JP); Kentaro FUJIWARA, Tokyo (JP); Shoutaro TSUJI, Yokohama-shi (JP)

(21) Appl. No.: 18/575,079

(22) PCT Filed: Jun. 30, 2022
(86) PCT No.: PCT/JP2022/026288

§ 371 Date: Nov. 5, 2024

(30) Foreign Application Priority Data
Jun. 30, 2021 (JP) 2021-108503

(51) Int. Cl. A61K 51/10; A61K 51/12

(52) U.S. Cl. CPC A61K 51/1054; A61K 51/12; A61K 2121/00; A61K 2123/00

(57) ABSTRACT

The present invention provides a humanized antibody that binds to a HEG1 protein and a complex of the antibody and a radionuclide. According to the present invention, there is a complex including an antibody, and a radionuclide, in which the antibody is a humanized antibody capable of binding a human HEG1 protein expressed on mesothelioma cells.

### TECHNICAL FIELD

The present disclosure provides a humanized antibody that binds to a HEG1 protein and a complex of the antibody and a radionuclide.

### BACKGROUND ART

Malignant mesothelioma is a disease that has become a major social problem as a malignant tumor that is primarily caused by exposure to asbestos. Early detection is difficult, and it is positioned as one of malignant tumors with poor prognosis. Malignant mesothelioma may be pathologically similar to metastatic adenocarcinoma, sarcoma, and reactive mesothelial cells, which are benign growths, and is often difficult to pathologically differentiate. In addition, it is not rare that various tissue types such as epithelial type and sarcoma type are taken, and diagnosis is difficult.

HEG1 protein has been shown to be effective as a marker for the diagnosis of malignant mesothelioma (Patent Literature 1). Patent Literature 1 discloses that mesothelioma can be detected with high sensitivity and specificity by reacting a mouse antibody that binds to a HEG1 protein with mesothelioma tissue.

### CITATION LIST

### Patent Literature

Patent Literature 1: WO2017/141604

### SUMMARY OF INVENTION

The present disclosure provides a humanized antibody that binds to a HEG1 protein and a complex of the antibody and a radionuclide.

The present inventors have developed a humanized antibody that binds to a HEG1 protein and a complex of the antibody and a radionuclide. The humanized antibodies are suitable for in vivo diagnostic or pharmaceutical applications.

An aspect of the present invention is a complex comprising an antibody or antigen-binding fragment thereof and a radionuclide, in which the antibody is a humanized antibody capable of binding a human HEG1 protein expressed on mesothelioma cells.

Another aspect of the present invention is a composition containing the complex described above.

### BRIEF DESCRIPTION OF DRAWINGS

FIG. 1 illustrates a binding activity of a humanized antibody including a combination of a humanized heavy chain variable region and a humanized light chain variable region to HEG1. Heavy and light chain information of the antibodies used accompanies each bar. Activity has been compared to murine antibodies (parental antibodies) with a murine heavy chain variable region (xiH) and a murine light chain variable region (xiL).

FIG. 2 illustrates the binding activity of the humanized antibody including the combination of the humanized heavy chain variable region and the humanized light chain variable region to HEG1. Heavy and light chain information of the antibodies used accompanies each bar. (-) indicates background in an absence of the antibodies.

FIG. 3 illustrates the binding activity of the humanized antibody including the combination of the humanized heavy chain variable region and the humanized light chain variable region to HEG1. Heavy and light chain information of the antibodies used accompanies each bar.

FIG. 4 illustrates the binding activity of the humanized antibody including the combination of the humanized heavy chain variable region and the humanized light chain variable region to HEG1. Heavy and light chain information of the antibodies used accompanies each bar.

FIG. 5 illustrates the binding activity of the humanized antibody including the combination of the humanized heavy chain variable region and the humanized light chain variable region to HEG1. Heavy and light chain information of the antibodies used accompanies each bar.

FIG. 6 illustrates the binding activity of the humanized antibody including the combination of the humanized heavy chain variable region and the humanized light chain variable region to HEG1. Heavy and light chain information of the antibodies used accompanies each bar.

FIG. 7 illustrates an influence of a signal peptide on a production amount of each humanized antibody.

FIG. 8 is a diagram illustrating a result of SDS-PAGE of each antibody purified from a culture supernatant by expressing each heavy chain and light chain in a CHO cell system.

FIG. 9 illustrates results of Biacore measurement of an affinity of each antibody of purified humanized SKM9-2 to a synthetic sugar epitope in a left column, and results of flow cytometry measurement of the binding of each antibody of purified humanized SKM9-2 to a mesothelioma cell line NCI-H226 in a right column.

FIG. 10 is a graph illustrating results of evaluating the binding and specificity of an ⁸⁹Zr complex labeled antibody to HEG1-positive and negative cells. A vertical axis represents a value (defined as Uptake ratio (% addition amount/mg)) obtained by correcting the proportion of the radioactivity bound to the cell from the radioactivity added to each well according to the protein mass in the well.

FIG. 11 is a graph illustrating the binding of a ²²⁵Ac complex labeled antibody to tumor sections.

FIG. 12 is a graph illustrating the binding of the ²²⁵Ac complex labeled antibody to NCI-H226, ACC-MESO4, and A549. Unlabeled SKM9-2 was added as a competing compound.

FIG. 13 is a graph illustrating a cell-killing effect of the ²²⁵Ac complex labeled antibody on NCI-H226 cells and ACC-MESO4 cells. As a comparative control, unlabeled SKM9-2 was added.

FIG. 14 is a representative example of PET images of tumor-bearing mice to which NCI-H226 cells had been transplanted and to which an ⁸⁹Zr complex labeled antibody had been administered.

### DESCRIPTION OF EMBODIMENTS

### Definitions

In the present specification, the “subject” may be a mammal, and examples thereof include primates such as humans, chimpanzees, and marmosets, experimental animals such as rats, mice, and rabbits, livestock animals such as pigs, cows, horses, sheep, and goats, and pet animals such as dogs and cats, but the “subject” is preferably a human. More preferably, the subject may be a “cancer patient” suffering from or at risk of a tumor or cancer. More preferably, the subject may be a subject suffering from or having the possibility of having mesothelioma. “Patient” means a subject suffering from cancer and is preferably, but not limited to, a human.

In the present specification, the “antibody” means an immunoglobulin, and refers to a protein having a structure in which two heavy chains (H chains) and two light chains (L chains) stabilized by disulfide bonds are associated with each other. The heavy chain consists of a heavy chain variable region VH, heavy chain constant regions CH1, CH2, and CH3, and a hinge region located between CH1 and CH2, and the light chain consists of a light chain variable region VL and a light chain constant region CL. Among them, the variable region fragment (Fv) composed of VH and VL is a region directly involved in antigen binding and giving diversity to the antibody. In addition, an antigen binding region composed of VL, CL, VH, and CH1 is referred to as a Fab region, and a region composed of a hinge region, CH2, and CH3 is referred to as an Fc region.

Among the variable regions, a region that comes into direct contact with an antigen has a particularly large variation, and is called a complementarity-determining region (CDR). A portion having a relatively small variation other than the CDR is referred to as a framework region (FR). Three CDRs exist in each of the variable regions of the light chain and the heavy chain, and are referred to as heavy chain CDRs 1 to 3 and light chain CDRs 1 to 3 in order from the N-terminal side. Each CDR is incorporated into a framework region. The heavy chain variable region of the antibody has, from the N-terminal side to the C-terminal side, a heavy chain framework region 1, a heavy chain CDR1, a heavy chain framework region 2, a heavy chain CDR2, a heavy chain framework region 3, a heavy chain CDR3, and a heavy chain framework region 4 in this order. The light chain variable region of the antibody has, from the N-terminal side to the C-terminal side, a light chain framework region 1, a light chain CDR1, a light chain framework region 2, a light chain CDR2, a light chain framework region 3, a light chain CDR3, and a light chain framework region 4 in this order. The antibody may be a recombinant protein (recombinant antibody) and may be produced in an animal cell, for example, a Chinese hamster ovary cell (CHO cell). The origin of the antibody is not particularly limited, and examples thereof include a non-human animal antibody, a non-human mammal antibody (for example, mouse antibody, rat antibody, camel antibody), and a human antibody. The antibody may also be a chimeric antibody, a humanized antibody, and a fully humanized antibody. The antibody may be a polyclonal antibody or a monoclonal antibody, and is preferably a monoclonal antibody. The “chimeric antibody” is an antibody in which a heavy chain constant region and a light chain constant region of different species are linked to a heavy chain variable region and a light chain variable region, respectively. The humanized antibody means an antibody in which the corresponding position of a human antibody is substituted with an amino acid sequence characteristic of a non-human-derived antibody, and examples thereof include an antibody having the heavy chain CDRs 1 to 3 and the light chain CDRs 1 to 3 of an antibody produced by immunizing a mouse or a rat, in which all other regions including four framework regions (FR) of each of the heavy chain and the light chain are derived from a human antibody. Such antibodies may also be referred to as CDR-grafted antibodies. A “humanized antibody” may also include a “human chimeric antibody”. A “human chimeric antibody” is an antibody in which the constant region of a non-human-derived antibody is substituted with the constant region of a human antibody in a non-human-derived antibody. In the present invention, an antibody is complexed with a radionuclide to form a conjugate complex of the antibody and the radionuclide. The radionuclide is preferably a metal radionuclide, for example a therapeutic metal radionuclide or a diagnostic metal radionuclide. In addition, the antibody may be a bispecific antibody. The antibody may be an isolated antibody or a purified antibody. The antibody can be, for example, IgG. The antibody can be, for example, IgG1, IgG2 (For example, IgG2a and IgG2b), IgG3, or IgG4.

The variable regions of immunoglobulin chains generally exhibit the same overall structure, including a relatively conserved framework region (FR) linked by three hypervariable regions (more often referred to as “complementarity determining regions” or CDRs). The CDRs obtained from the two chains of each of the above heavy/light chain pairs are typically juxtaposed by framework regions to form a structure that specifically binds a specific epitope on a target protein (for example, PCSK9). From the N-terminal side to the C-terminal side, any naturally occurring light and heavy chain variable regions typically conform to the following sequence of these elements. FR1, CDR1, FR2, CDR2, FR3, CDR3 and FR4. Numbering systems have been devised to assign numbers to the amino acids occupying positions in each of these domains. This numbering system is defined in “Kabat Sequences of Proteins of Immunological Interest (1987 and 1991, NIH, Bethesda, MD)” or “Chothia & Lesk, 1987, J. Mol. Biol. 196:901-917; Chothia et al., 1989, Nature 342:878-883”.

In the present specification, a “naked antibody” refers to an antibody prior to being subjected to a process of complexing with a linker or a radionuclide.

In the present specification, the term “antigen-binding fragment” means a part of an antibody that maintains binding to an antigen. The antigen-binding fragment may include a heavy chain variable region or a light chain variable region, or both, of an antibody of the present disclosure. The antigen-binding fragment may be chimerized or humanized. Examples of the antigen-binding fragment include Fab, Fab′, F(ab′)₂, and Fv. In addition, the antibody-binding fragment may also include recombinantly produced conjugates or functional equivalents (for example, some other antibodies having the form of scFv (single chain Fv), diabody, scDb, tandem scFv, leucine zipper type, sc(Fv)₂ (single chain (Fv)₂), or the like). The antigen-binding fragment of such an antibody is not particularly limited, but can be obtained, for example, by treating the antibody with an enzyme. For example, the antibody can be digested with papain to obtain yield Fab. Alternatively, the antibody can be digested with pepsin to yield F(ab′)₂, which can be further reduced to yield Fab′. In the present specification, the antigen-binding fragment of such antibodies can be used. In scFv, VL and VH can be linked with an artificial polypeptide linker to maintain the same antigen specificity as the original antibody. VL and VH may be linked from the N-terminal side in the order of VH and VL or VL and VH. The linker may have a length of the order of 10 to 25 amino acids. The linker may be rich in glycine and may contain an amino acid such as serine or threonine for the purpose of enhancing water solubility.

In the present specification, the “HEG1 protein” is a protein expressed in the membrane of mesothelioma cells (WO2017/141604). According to WO2017/141604, it is considered that the HEG1 protein is subjected to sugar modification on the membrane of mesothelioma cells. The sugar chain modification includes O-type sugar chain modification. The sugar chain modification is sialylated. The sugar chain modification may include α2,3 sialylation. The sugar modification is considered to be mesothelioma-specific. Therefore, according to WO2017/141604, mesothelioma cells can be detected by an antibody that binds to a HEG1 protein having this sugar modification. Examples of the human HEG1 proteins include proteins having a nucleic acid sequence registered as NM_020733.1 at the National Center for Biotechnology Information (NCBI) and the amino acid sequence encoded thereby. From the result of gene ontology analysis, it is predicted that in the HEG1 protein, a signal peptide moiety will be a domain corresponding to positions 1 to 29 of the amino acid sequence, an extracellular domain will be a domain corresponding to positions 30 to 1248 of the amino acid sequence, a transmembrane domain is a domain corresponding to positions 1249 to 1269 of the amino acid sequence, and an intracellular domain is a domain corresponding to positions 1270 to 1381 of the amino acid sequence. For example, the HEG1 protein may also include a protein having an amino acid sequence 90% or more, 95% or more, 98% or more, or 99% or more homologous to the above-described amino acid sequence. For example, the HEG1 protein may contain one or more amino acid substitutions, insertions, additions and/or deletions in the amino acid sequence represented by the amino acid sequence.

In the present specification, the amino acid sequence is represented by a one-letter code. That is, A represents alanine, R represents arginine, N represents asparagine, D represents aspartic acid, C represents cysteine, Q represents glutamine, E represents glutamic acid, G represents glycine, H represents histidine, I represents isoleucine, L represents leucine, K represents lysine, M represents methionine, F represents phenylalanine, P represents proline, S represents serine, T represents threonine, W represents tryptophan, Y represents tyrosine, and V represents valine.

In the present specification, “mesothelioma” means a tumor derived from mesothelial cells. As the mesothelioma, pleural mesothelioma, peritoneal mesothelioma, pericardial mesothelioma, and testicular sheath mesothelioma are known from the generation site thereof. In the present specification, mesothelioma means benign mesothelioma and/or malignant mesothelioma. Mesothelioma is roughly classified into epithelial mesothelioma, sarcomatous mesothelioma, biphasic mesothelioma, and other mesotheliomas (fibrogenic type, or the like) from the tissue type. In certain aspects, the mesothelioma can be malignant mesothelioma.

### <Complex (Conjugate) of Antibody and Radionuclide of Present Disclosure>

The complex of the antibody and the radionuclide may be used to target the radionuclide to a target antigen in a living body. In a case where the radionuclide is the diagnostic metal radionuclide, the distribution of the target antigen in the living body can be analyzed via a signal (radioactivity) emitted from the metal radionuclide. In a case where the radionuclide is the therapeutic metal radionuclide, the radionuclide is targeted to a target antigen in vivo, and delivered to the target antigen can kill cells (in particular, cells expressing the target antigen, such as cancer cells) in the vicinity of the target antigen. In this way, complexing the radionuclide with the antibody has utility.

According to the present disclosure, a complex of an antibody and a radionuclide (antibody-RI conjugate) is provided. In the complex of the antibody and the radionuclide according to the present disclosure, the following humanized antibodies can be used as the antibody. Accordingly, the present disclosure provides the complex of the humanized antibody and the radionuclide.

In the present disclosure, the antibody is an antibody that binds to a HEG1 protein. The HEG1 protein may be the HEG1 protein expressed in mesothelioma cells (for example, ACC-MESO4 cell line) (WO2017/141604). The HEG1 protein expressed in mesothelioma cells may include mesothelioma-specific sugar chain modifications. Since the sugar chain modification is decomposed by α2,3-neuraminidase treatment, it includes α2,3-sialylation (WO2017/141604). In addition, the sugar chain modification may be an O-type sugar chain modification because it is not degraded by N-glycanase (PNGase F) (WO2017/141604).

In certain preferred aspects, the antibodies of the present disclosure bind to the HEG1 protein expressed in the mesothelioma cells. In certain preferred aspects, the antibody binds to the HEG1 protein in a manner dependent on an O-type sugar chain modification containing α2,3 sialic acid. That is, the antibody can partially or completely lose binding to the HEG1 protein by α2,3 neuraminidase treatment. The antibody can also partially o completely lose binding to HEG1 protein by proteinase K treatment.

According to the present disclosure, the radionuclide may be directly linked to the antibody in the complex of the antibody and the radionuclide. According to the present disclosure, in the complex of the antibody and the radionuclide, the antibody and the radionuclide may be linked via a linker. In this case, the radionuclide is used as a metal radionuclide, and the metal radionuclide may form a chelate (complex) with a chelating agent, and the antibody and the chelating agent may be linked via a linker or without a linker. Here, the linkage is preferably a covalent linkage.

### (1) Humanized Antibody of Present Disclosure

According to the present disclosure, the humanized antibody that binds to a HEG1 (particularly human HEG1) protein is provided. In certain preferred aspects, the antibodies of the present disclosure bind to the HEG1 protein expressed in the mesothelioma cells. In certain preferred aspects, the antibody binds to the HEG1 protein in a manner dependent on an O-type sugar chain modification containing α2,3 sialic acid. That is, the antibody can partially or completely lose binding to the HEG1 protein by α2,3 neuraminidase treatment. The antibody can also partially or completely lose binding to HEG1 protein by proteinase K treatment.

In certain aspects, the antibody of the present disclosure is an antibody that binds to a HEG1 protein expressed in mesothelioma cells (for example, ACC-MESO4 cell line). Such an antibody can be obtained by a conventional method, for example, as described in WO2017/141604. Specifically, for example, the antibody binds to a HEG1 protein expressed in mesothelioma cells (for example, ACC-MESO4 cell line), but in certain aspects, the binding can be eliminated or reduced by protease K treatment of the HEG1 protein. For example, the antibody binds to the HEG1 protein expressed in mesothelioma cells (for example, ACC-MESO4 cell line), but in certain aspects, the binding can be eliminated or reduced by α2,3 neuraminidase treatment. In certain aspects, the binding is not eliminated by treatment with one or more or any selected from the group consisting of β-N-acetylglucosaminidase, N-glycanase (PNGaseF), lysozyme, and hyaluronidase. The processing is performed under conditions suitable for the individual processing. In certain aspects, the antibody can be a human antibody. The human antibody can be made by immunizing a non-human mammal (for example, mouse) incorporating human IgG loci with an immunogen. In certain aspects, a human heavy chain variable region is inserted upstream of the heavy chain constant region of mouse IgG, and a mouse having a human light chain variable region inserted upstream of the light chain constant region of mouse IgG is immunized with an immunogen to obtain an antibody as a human chimeric antibody, and the constant region can be replaced with a corresponding human constant region (see, for example, WO2002/066630A and WO2011/004192A.). The humanized antibody can be generated by removing the six CDRs of a human antibody and replacing them with the corresponding CDRs (six CDRs) of the resulting antibody.

In certain aspects, an antibody or antigen-binding fragment thereof of the present disclosure is a humanized antibody that binds to a partial peptide of a HEG1 (particularly human HEG1) protein. A partial peptide can be, for example, an antibody which binds to a peptide having an amino acid sequence set forth in SEQ ID NO: 182 (SKSPSLVSLPT). The partial peptide can be, for example, a peptide produced by mesothelioma cells (for example, ACC-MESO4 cell line). The peptide can be obtained as a fusion protein by being linked to the N-terminal side of a protein (SEQ ID NO: 183; hereinafter, referred to as “SLURPgpi”; the signal sequence is shown in SEQ ID NO: 184) in which a GPI anchor signal is linked to the N-terminal of human SLURP1, for example, and can be used for evaluation of binding to an antibody. In certain preferred aspects, in the peptide having an amino acid sequence set forth in SEQ ID NO: 182 (SKSPSLVSLPT), either or both of the first serine and the eighth serine have an O-type sugar chain modification. In certain preferred aspects, the O-type sugar chain modification can be a 2,3-sialyl T antigen (2,3-Sialyl T), or a disialyl T antigen (DiSialyl T). In a most preferred aspect, there is provided an antibody that is for an antigen having an amino acid sequence set forth in SEQ ID NO: 182 (SKSPSLVSLPT), in which either or both of the first serine and the eighth serine have a sugar chain modification, and the sugar chain modification is 2,3-sialyl T antigen (2,3-Sialyl T) or disialyl T antigen (DiSialyl T).

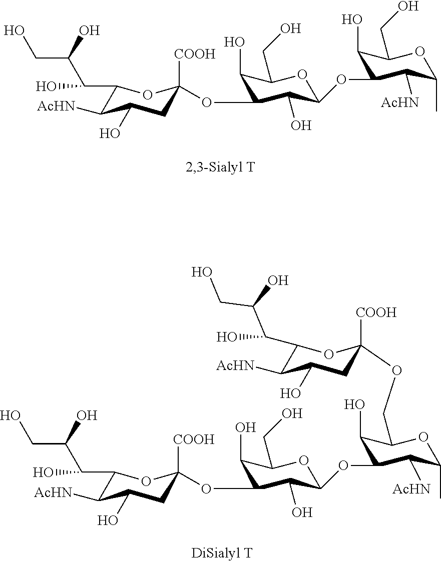

In certain aspects, a humanized antibody of the present disclosure may include a heavy chain variable region including a heavy chain CDR1 having an amino acid sequence set forth in SEQ ID NO: 37, a heavy chain CDR2 having an amino acid sequence set forth in SEQ ID NO: 43, and a heavy chain CDR3 having an amino acid sequence set forth in SEQ ID NO: 51, and a light chain variable region including a light chain CDR1 having an amino acid sequence set forth in SEQ ID NO: 62, a light chain CDR2 having an amino acid sequence set forth in SEQ ID NO: 75, and a light chain CDR3 having an amino acid sequence set forth in SEQ ID NO: 82.

In certain aspects, a humanized antibody of the present disclosure may include a heavy chain variable region including a heavy chain CDR1 having the amino acid sequence set forth in SEQ ID NO: 37, a heavy chain CDR2 having the amino acid sequence set forth in SEQ ID NO: 43, and a heavy chain CDR3 having the amino acid sequence set forth in SEQ ID NO: 51, and a light chain variable region including a light chain CDR1 having the amino acid sequence set forth in SEQ ID NO: 63, a light chain CDR2 having the amino acid sequence set forth in SEQ ID NO: 75, and a light chain CDR3 having the amino acid sequence set forth in SEQ ID NO: 82.

In certain aspects, the humanized antibody of the present disclosure may include a heavy chain variable region having any of the following heavy chain CDRs 1 to 3 sets (1A) to (8A):
(1A) a heavy chain CDR1 having the amino acid sequence set forth in SEQ ID NO: 89, a heavy chain CDR2 having the amino acid sequence set forth in SEQ ID NO: 90, and a heavy chain CDR3 having the amino acid sequence set forth in SEQ ID NO: 91;
(2A) a heavy chain CDR1 having the amino acid sequence set forth in SEQ ID NO: 92, a heavy chain CDR2 having the amino acid sequence set forth in SEQ ID NO: 93, and a heavy chain CDR3 having the amino acid sequence set forth in SEQ ID NO: 94;
(3A) a heavy chain CDR1 having the amino acid sequence set forth in SEQ ID NO: 95, a heavy chain CDR2 having the amino acid sequence set forth in SEQ ID NO: 96, and a heavy chain CDR3 having the amino acid sequence set forth in SEQ ID NO: 97;
(4A) a heavy chain CDR1 having the amino acid sequence set forth in SEQ ID NO: 98, a heavy chain CDR2 having the amino acid sequence set forth in SEQ ID NO: 99, and a heavy chain CDR3 having the amino acid sequence set forth in SEQ ID NO: 100;
(5A) a heavy chain CDR1 having the amino acid sequence set forth in SEQ ID NO: 101, a heavy chain CDR2 having the amino acid sequence set forth in SEQ ID NO: 102, and a heavy chain CDR3 having the amino acid sequence set forth in SEQ ID NO: 103;
(6A) a heavy chain CDR1 having the amino acid sequence set forth in SEQ ID NO: 104, a heavy chain CDR2 having the amino acid sequence set forth in SEQ ID NO: 105, and a heavy chain CDR3 having the amino acid sequence set forth in SEQ ID NO: 106;
(7A) a heavy chain CDR1 having the amino acid sequence set forth in SEQ ID NO: 110, a heavy chain CDR2 having the amino acid sequence set forth in SEQ ID NO: 111, and a heavy chain CDR3 having the amino acid sequence set forth in SEQ ID NO: 112; and
(8A) a heavy chain CDR1 having the amino acid sequence set forth in SEQ ID NO: 113, a heavy chain CDR2 having the amino acid sequence set forth in SEQ ID NO: 114, and a heavy chain CDR3 having the amino acid sequence set forth in SEQ ID NO: 115.

The humanized antibody of the present disclosure may include a heavy chain variable region including the heavy chain CDRs 1 to 3 of any of (1A) to (8A) above.

In certain aspects, the humanized antibody of the present disclosure may include a light chain variable region having any of the following light chain CDRs 1 to 3 sets (1B) to (18B):
(1B) a light chain CDR1 having the amino acid sequence set forth in SEQ ID NO: 116, a light chain CDR2 having the amino acid sequence set forth in SEQ ID NO: 117, and a light chain CDR3 having the amino acid sequence set forth in SEQ ID NO: 118;
(2B) a light chain CDR1 having the amino acid sequence set forth in SEQ ID NO: 119, a light chain CDR2 having the amino acid sequence set forth in SEQ ID NO: 120, and a light chain CDR3 having the amino acid sequence set forth in SEQ ID NO: 121;
(3B) a light chain CDR1 having the amino acid sequence set forth in SEQ ID NO: 122, a light chain CDR2 having the amino acid sequence set forth in SEQ ID NO: 123, and a light chain CDR3 having the amino acid sequence set forth in SEQ ID NO: 124;
(4B) a light chain CDR1 having the amino acid sequence set forth in SEQ ID NO: 128, a light chain CDR2 having the amino acid sequence set forth in SEQ ID NO: 129, and a light chain CDR3 having the amino acid sequence set forth in SEQ ID NO: 130;
(5B) a light chain CDR1 having the amino acid sequence set forth in SEQ ID NO: 131, a light chain CDR2 having the amino acid sequence set forth in SEQ ID NO: 132, and a light chain CDR3 having the amino acid sequence set forth in SEQ ID NO: 133;
(6B) a light chain CDR1 having the amino acid sequence set forth in SEQ ID NO: 134, a light chain CDR2 having the amino acid sequence set forth in SEQ ID NO: 135, and a light chain CDR3 having the amino acid sequence set forth in SEQ ID NO: 136;
(7B) a light chain CDR1 having the amino acid sequence set forth in SEQ ID NO: 137, a light chain CDR2 having the amino acid sequence set forth in SEQ ID NO: 138, and a light chain CDR3 having the amino acid sequence set forth in SEQ ID NO: 139;
(8B) a light chain CDR1 having the amino acid sequence set forth in SEQ ID NO: 140, a light chain CDR2 having the amino acid sequence set forth in SEQ ID NO: 141, and a light chain CDR3 having the amino acid sequence set forth in SEQ ID NO: 142;
(9B) a light chain CDR1 having the amino acid sequence set forth in SEQ ID NO: 143, a light chain CDR2 having the amino acid sequence set forth in SEQ ID NO: 144, and a light chain CDR3 having the amino acid sequence set forth in SEQ ID NO: 145;
(10B) a light chain CDR1 having the amino acid sequence set forth in SEQ ID NO: 146, a light chain CDR2 having the amino acid sequence set forth in SEQ ID NO: 147, and a light chain CDR3 having the amino acid sequence set forth in SEQ ID NO: 148;
(11B) a light chain CDR1 having the amino acid sequence set forth in SEQ ID NO: 149, a light chain CDR2 having the amino acid sequence set forth in SEQ ID NO: 150, and a light chain CDR3 having the amino acid sequence set forth in SEQ ID NO: 151;
(12B) a light chain CDR1 having the amino acid sequence set forth in SEQ ID NO: 152, a light chain CDR2 having the amino acid sequence set forth in SEQ ID NO: 153, and a light chain CDR3 having the amino acid sequence set forth in SEQ ID NO: 154;
(13B) a light chain CDR1 having the amino acid sequence set forth in SEQ ID NO: 155, a light chain CDR2 having the amino acid sequence set forth in SEQ ID NO: 156, and a light chain CDR3 having the amino acid sequence set forth in SEQ ID NO: 157;
(14B) a light chain CDR1 having the amino acid sequence set forth in SEQ ID NO: 158, a light chain CDR2 having the amino acid sequence set forth in SEQ ID NO: 159, and a light chain CDR3 having the amino acid sequence set forth in SEQ ID NO: 160;
(15B) a light chain CDR1 having the amino acid sequence set forth in SEQ ID NO: 161, a light chain CDR2 having the amino acid sequence set forth in SEQ ID NO: 162, and a light chain CDR3 having the amino acid sequence set forth in SEQ ID NO: 163;
(16B) a light chain CDR1 having the amino acid sequence set forth in SEQ ID NO: 173, a light chain CDR2 having the amino acid sequence set forth in SEQ ID NO: 174, and a light chain CDR3 having the amino acid sequence set forth in SEQ ID NO: 175;
(17B) a light chain CDR1 having the amino acid sequence set forth in SEQ ID NO: 176, a light chain CDR2 having the amino acid sequence set forth in SEQ ID NO: 177, and a light chain CDR3 having the amino acid sequence set forth in SEQ ID NO: 178; and
(18B) a light chain CDR1 having the amino acid sequence set forth in SEQ ID NO: 179, a light chain CDR2 having the amino acid sequence set forth in SEQ ID NO: 180, and a light chain CDR3 having the amino acid sequence set forth in SEQ ID NO: 181.

The humanized antibody of the present disclosure may include a light chain variable region including the light chain CDRs 1 to 3 of any of (1B) to (18B) above.

In certain aspects, an antibody of the present disclosure may include
a heavy chain variable region including a heavy chain CDR1 having the amino acid sequence set forth in SEQ ID NO: 37, a heavy chain CDR2 having the amino acid sequence set forth in SEQ ID NO: 43, and a heavy chain CDR3 having the amino acid sequence set forth in SEQ ID NO: 51, and
a light chain variable region including the set of light chain CDRs 1 to 3 of any of (1B) to (18B) above.

In certain aspects, an antibody of the present disclosure may include
a heavy chain variable region including the set of heavy chain CDRs 1 to 3 of any of (1A) to (8A) above; and
a light chain variable region including a light chain CDR1 having the amino acid sequence set forth in SEQ ID NO: 62, a light chain CDR2 having the amino acid sequence set forth in SEQ ID NO: 75, and a light chain CDR3 having the amino acid sequence set forth in SEQ ID NO: 82. Here, the light chain CDR1 may preferably have the amino acid sequence set forth in SEQ ID NO: 63.

In certain aspects, an antibody of the present disclosure may include:
a heavy chain variable region including the set of heavy chain CDRs 1 to 3 of any of (1A) to (8A) above; and
a light chain variable region including the set of light chain CDRs 1 to 3 of any of (1B) to (18B) above.

In certain aspects, an antibody of the present disclosure may include:
a heavy chain variable region including the set of heavy chain CDRs 1 to 3 of (1A) above; and
a light chain variable region including the set of light chain CDRs 1 to 3 of any of (1B) to (18B) above. In certain preferred aspects, the light chain variable region may include the set of light chain CDRs 1 to 3 of (1B) above. In certain preferred aspects, the light chain variable region may include the set of light chain CDRs 1 to 3 of any of (2B) to (18B) above. In certain preferred aspects, the light chain variable region may include the set of light chain CDRs 1 to 3 of any of (2B) to (10B) above. In certain preferred aspects, the light chain variable region may include the set of light chain CDRs 1 to 3 of any of (2B) to (7B) above and (10B) (particularly (4B) or (10B)). In certain preferred aspects, the light chain variable region can be (10B) above.

In certain aspects, an antibody of the present disclosure may include:
a heavy chain variable region including the set of heavy chain CDRs 1 to 3 of (2A) above; and
a light chain variable region including the set of light chain CDRs 1 to 3 of any of (1B) to (18B) above. In certain preferred aspects, the light chain variable region may include the set of light chain CDRs 1 to 3 of (1B) above. In certain preferred aspects, the light chain variable region may include the set of light chain CDRs 1 to 3 of any of (2B) to (18B) above. In certain preferred aspects, the light chain variable region may include the set of light chain CDRs 1 to 3 of any of (2B) to (10B) above. In certain preferred aspects, the light chain variable region may include the set of light chain CDRs 1 to 3 of any of (2B) to (7B) above and (10B) (particularly (4B) or (10B)). In certain preferred aspects, the light chain variable region can be (10B) above.

In certain aspects, an antibody of the present disclosure may include:
a heavy chain variable region including the set of heavy chain CDRs 1 to 3 of (3A) above; and
a light chain variable region including the set of light chain CDRs 1 to 3 of any of (1B) to (18B) above. In certain preferred aspects, the light chain variable region may include the set of light chain CDRs 1 to 3 of (1B) above. In certain preferred aspects, the light chain variable region may include the set of light chain CDRs 1 to 3 of any of (2B) to (18B) above. In certain preferred aspects, the light chain variable region may include the set of light chain CDRs 1 to 3 of any of (2B) to (10B) above. In certain preferred aspects, the light chain variable region may include the set of light chain CDRs 1 to 3 of any of (2B) to (7B) above and (10B) (particularly (4B) or (10B)). In certain preferred aspects, the light chain variable region can be (10B) above.

In certain aspects, an antibody of the present disclosure may include:
a heavy chain variable region including the set of heavy chain CDRs 1 to 3 of (4A) above; and
a light chain variable region including the set of light chain CDRs 1 to 3 of any of (1B) to (18B) above. In certain preferred aspects, the light chain variable region may include the set of light chain CDRs 1 to 3 of (1B) above. In certain preferred aspects, the light chain variable region may include the set of light chain CDRs 1 to 3 of any of (2B) to (18B) above. In certain preferred aspects, the light chain variable region may include the set of light chain CDRs 1 to 3 of any of (2B) to (10B) above. In certain preferred aspects, the light chain variable region may include the set of light chain CDRs 1 to 3 of any of (2B) to (7B) above and (10B) (particularly (4B) or (10B)). In certain preferred aspects, the light chain variable region can be (10B) above.

In certain aspects, an antibody of the present disclosure may include:
a heavy chain variable region including the set of heavy chain CDRs 1 to 3 of (5A) above; and
a light chain variable region including the set of light chain CDRs 1 to 3 of any of (1B) to (18B) above. In certain preferred aspects, the light chain variable region may include the set of light chain CDRs 1 to 3 of (1B) above. In certain preferred aspects, the light chain variable region may include the set of light chain CDRs 1 to 3 of any of (2B) to (18B) above. In certain preferred aspects, the light chain variable region may include the set of light chain CDRs 1 to 3 of any of (2B) to (10B) above. In certain preferred aspects, the light chain variable region may include the set of light chain CDRs 1 to 3 of any of (2B) to (7B) and (10B) above (in particular any of (4B), (7B), (10B) or (14B) to (16B)). In certain preferred aspects, the light chain variable region can be (10B) above.

In certain aspects, an antibody of the present disclosure may include:
a heavy chain variable region including the set of heavy chain CDRs 1 to 3 of (6A) above; and
a light chain variable region including the set of light chain CDRs 1 to 3 of any of (1B) to (18B) above. In certain preferred aspects, the light chain variable region may include the set of light chain CDRs 1 to 3 of (1B) above. In certain preferred aspects, the light chain variable region may include the set of light chain CDRs 1 to 3 of any of (2B) to (18B) above. In certain preferred aspects, the light chain variable region may include the set of light chain CDRs 1 to 3 of any of (2B) to (10B) above. In certain preferred aspects, the light chain variable region may include the set of light chain CDRs 1 to 3 of any of (2B) to (7B) and (10B) above (in particular any of (4B), (7B), (10B) or (14B) to (16B)). In certain preferred aspects, the light chain variable region can be (10B) above.

In certain aspects, an antibody of the present disclosure may include:
a heavy chain variable region including the set of heavy chain CDRs 1 to 3 of (7A) above; and
a light chain variable region including the set of light chain CDRs 1 to 3 of any of (1B) to (18B) above. In certain preferred aspects, the light chain variable region may include the set of light chain CDRs 1 to 3 of (1B) above. In certain preferred aspects, the light chain variable region may include the set of light chain CDRs 1 to 3 of any of (2B) to (18B) above. In certain preferred aspects, the light chain variable region may include the set of light chain CDRs 1 to 3 of any of (2B) to (10B) above. In certain preferred aspects, the light chain variable region may include the set of light chain CDRs 1 to 3 of any of (2B) to (7B) above and (10B) (particularly (4B) or (10B)). In certain preferred aspects, the light chain variable region can be (10B) above.

In certain aspects, an antibody of the present disclosure may include:
a heavy chain variable region including the set of heavy chain CDRs 1 to 3 of (8A) above; and
a light chain variable region including the set of light chain CDRs 1 to 3 of any of (1B) to (18B) above. In certain preferred aspects, the light chain variable region may include the set of light chain CDRs 1 to 3 of (1B) above. In certain preferred aspects, the light chain variable region may include the set of light chain CDRs 1 to 3 of any of (2B) to (18B) above. In certain preferred aspects, the light chain variable region may include the set of light chain CDRs 1 to 3 of any of (2B) to (10B) above. In certain preferred aspects, the light chain variable region may include the set of light chain CDRs 1 to 3 of any of (2B) to (7B) above and (10B) (particularly (4B) or (10B)). In certain preferred aspects, the light chain variable region can be (10B) above.

In certain preferred aspects, in the antibody of the present disclosure, the variable region of the antibody to which the heavy chain CDRs 1 to 3 and the light chain CDRs 1 to 3 are grafted may be a variable region of a human IgG1 antibody. In certain aspects, in the antibody of the present disclosure, the variable region of the antibody to which the heavy chain CDRs 1 to 3 and the light chain CDRs 1 to 3 are grafted may be a variable region of a human IgG2 antibody. In certain aspects, in the antibody of the present disclosure, the variable region of the antibody to which the heavy chain CDRs 1 to 3 and the light chain CDRs 1 to 3 are grafted may be a variable region of a human IgG3 antibody. In certain aspects, in the antibody of the present disclosure, the variable region of the antibody to which the heavy chain CDRs 1 to 3 and the light chain CDRs 1 to 3 are grafted may be a variable region of a human IgG4 antibody.

In certain preferred aspects, in the antibody of the present disclosure, the Fc region (that is, heavy chain constant regions 2 and/or 3) may be the Fc region of the human IgG1 antibody. In certain preferred aspects, in the antibody of the present disclosure, the Fc region (that is, heavy chain constant regions 2 and/or 3) may be the Fc region of the human IgG2 antibody. In certain preferred aspects, in the antibody of the present disclosure, the Fc region (that is, heavy chain constant regions 2 and/or 3) may be the Fc region of the human IgG3 antibody. In certain preferred aspects, in the antibody of the present disclosure, the Fc region (that is, heavy chain constant regions 2 and/or 3) may be the Fc region of the human IgG4 antibody.

In certain aspects, in the antibody of the present disclosure, the heavy chain variable region may include
a framework region 1 having the amino acid sequence set forth in SEQ ID NO: 32, a framework region 2 having the amino acid sequence set forth in SEQ ID NO: 40, a framework region 3 having the amino acid sequence set forth in SEQ ID NO: 46, and a framework region 4 having the amino acid sequence set forth in SEQ ID NO: 54.

In certain preferred aspects, in the antibody of the present disclosure, the heavy chain variable region includes a framework region 1 having the amino acid sequence set forth in SEQ ID NO: 33, a framework region 2 having the amino acid sequence set forth in SEQ ID NO: 40, a framework region 3 having the amino acid sequence set forth in SEQ ID NO: 46, and a framework region 4 having the amino acid sequence set forth in SEQ ID NO: 54. Wherein, in SEQ ID NO: 46, X1 may be A, X3 may be T, X5 may be E, X6 may be R, and X7 may be T, and in SEQ ID NO: 54, X1 may be V, X3 may be T, X4 may be Q or E, and X5 may be P.

In certain aspects, in the antibody of the present disclosure, the heavy chain variable region may include a framework region 1 having the amino acid sequence set forth in SEQ ID NO: 33, a framework region 2 having the amino acid sequence set forth in SEQ ID NO: 42, a framework region 3 having the amino acid sequence set forth in SEQ ID NO: 48, and a framework region 4 having the amino acid sequence set forth in SEQ ID NO: 59.

In certain aspects, in the antibody of the present disclosure, the light chain variable region may include a framework region 1 having the amino acid sequence set forth in SEQ ID NO: 57, a framework region 2 having the amino acid sequence set forth in SEQ ID NO: 72, a framework region 3 having the amino acid sequence set forth in SEQ ID NO: 79, and a framework region 4 having the amino acid sequence set forth in SEQ ID NO: 86.

In certain aspects, in the antibody of the present disclosure, the light chain variable region may include a framework region 1 having the amino acid sequence set forth in SEQ ID NO: 61, a framework region 2 having the amino acid sequence set forth in SEQ ID NO: 74, a framework region 3 having the amino acid sequence set forth in SEQ ID NO: 81, and a framework region 4 having the amino acid sequence set forth in SEQ ID NO: 88.

In certain aspects, an antibody of the present disclosure may include one heavy chain variable region selected from the group consisting of SEQ ID NOS: 1 to 6, 8, and 9 and one light chain variable region selected from the group consisting of SEQ ID NOs: 10, 12 to 15, 17 to 21, 23 to 25, and 29 to 31. In certain aspects, the antibody of the present disclosure may include one heavy chain variable region selected from the group consisting of SEQ ID NOs: 2, 4 to 6, 8, and 9 and one light chain variable region selected from the group consisting of SEQ ID NOS: 14, 15, 17 to 21, 23 to 25, and 29 to 31. In certain preferred aspects, the antibody of the present disclosure may include a heavy chain variable region set forth in SEQ ID NO: 6 and a light chain variable region set forth in SEQ ID NO: 21.

The antibody or antigen-binding fragment thereof of the present disclosure may include an antibody or antigen-binding fragment thereof having a mutation selected from the group consisting of insertion, deletion, addition and substitution of one to several amino acids. In certain embodiments, provided is an antibody or an antigen-binding fragment thereof including at least one CDR, at least two, at least three, or more CDRs substantially identical to at least one CDR, at least two, at least three, or more CDRs in the antibody or the antigen-binding fragment thereof of the present disclosure. In another embodiment, an antibody having at least two, three, four, five, or six CDRs substantially identical to at least two, three, four, five, or six CDRs in or derived from the antibody or the antigen-binding fragment thereof of the present disclosure is included. In certain embodiments, at least one, two, or three CDRs in the antibody or the antigen-binding fragment thereof of the present disclosure and at least one, two, three, four, five, or six CDRs that are at least about 85%, 86%, 87%, 88%, 89%, 90%, 95%, 96%, 97%, 98%, or 99% identical are included. In certain embodiments, at least one, two, three, four, five, or six CDRs include at least one insertion, deletion, addition, or substitution in the antibody or the antigen-binding fragment thereof of the present disclosure or in at least one, two, three, four, five, or six CDRs derived from the antibody or the antigen-binding fragment thereof of the present disclosure. The antibody or the antigen-binding fragment thereof of the present disclosure may include an antibody or antigen-binding fragment thereof having an amino acid sequence identity of 80% or more, 85% or more, 90% or more, or 95% or more and having antigenic specificity of the antibody. The antibody or the antigen-binding fragment thereof of the present disclosure may include, for example, an antibody or an antigen-binding fragment thereof having 80% or more, 85% or more, 90% or more, or 95% or more amino acid sequence identity with the antibody disclosed above within a framework region and having antigen-specificity of the antibody. The antibody or the antigen-binding fragment thereof of the present disclosure may include, for example, an antibody or an antigen-binding fragment thereof having a mutation selected from the group consisting of insertion, deletion, addition and substitution of one to several amino acids in the antibody disclosed above within a framework region.

### (2) Radionuclide

According to the present disclosure, a complex of the above antibody and the radionuclide may be provided. The radionuclide may include a radiohalogen nuclide such as ¹⁸F, ¹²³I, ¹²⁴I, ¹²⁵I, ¹³¹I, or ²¹¹At, but the radiohalogen metal or the metal radionuclide exemplified by Al¹⁸F is preferable, and the metal radionuclide is more preferable, and may be the therapeutic metal radionuclide or diagnostic metal radionuclide.

The diagnostic metal radionuclide include ¹¹¹In (indium), ⁸⁹Zr (zirconium), ^{67/68}Ga (gallium), ^{99m}TC (technetium), ⁶⁴Cu (copper), preferably ¹¹¹In, and ⁸⁹Zr. For example, ¹¹¹In, ⁸⁹Zr, ⁶⁴Cu, ^{67/68}Ga, and ^{99m}Tc can be used for detection and diagnosis of cancer, for example, ⁸⁹Zr and ⁶⁴Cu can be used for positron emission tomography (PET), and ¹¹¹In and ^{99m}Tc can be used for single photon emission tomography (SPECT).

As the therapeutic metal radionuclide, it is preferable to use an x-ray emitting nuclide or a β-ray emitting nuclide from the viewpoint of enhancing the therapeutic effect. The x-ray emitting nuclide may be any nuclide that emits x rays in the disintegration process of the radioactive metal, and specifically, ²¹²Bi, ²¹³Bi, ²²⁷Th, ²²⁵Ac or the like is preferably used, more preferably ²²⁷Th or ²²⁵Ac, and still more preferably ²²⁵Ac. The β-ray emitting nuclide may be any nuclide that emits β rays in the disintegration process of a radioactive metal. Specifically, ⁶⁰Co, ⁵⁹Fe, ⁶⁴Cu, ⁶⁷Cu, ⁸⁹Sr, ⁹⁰Y, ^{99m}TC, ¹⁰³Ru, ¹⁵³Sm, ¹⁶⁵Dy, ¹⁶⁶Ho, ¹⁷⁷Lu, ¹⁸⁶Re, ¹⁸⁸Re, ¹⁹⁸Au, ²⁰³Hg, ²¹²Bi, ²¹³Bi, ²¹²Pb, or the like is preferably used, and ⁶⁴Cu, ⁶⁷Cu, ⁸⁹Sr, ⁹⁰Y, or ¹⁷⁷Lu is more preferably used. The x-ray emitting nuclide has a strong ionization action and a weak transmission force, so that the x-ray emitting nuclide can intensively exert a strong ionization action at the delivered site. Therefore, it can be particularly preferably used in the treatment of cancer from the viewpoint of minimizing the exposure to a site other than the delivered site and from the viewpoint of exerting a strong therapeutic effect.

In the present disclosure, the antibody and the radionuclide may be directly linked, or may be linked via a linker. In certain aspects, the antibody and the metal radionuclide may be linked via a linker, and the linker has a chelate site to which the metal radionuclide may be chelated.

### (3) Chelate Site

In the present disclosure, as the chelate site, a chelating agent capable of chelating a metal radionuclide or a metal halogenated with a radiohalogen nuclide (radiohalogen metal) can be used. Such chelating agents are not particularly limited, and examples thereof include diethylenetriaminepentaacetic acid (DTPA), deferoxamine, 1,4,7,10-tetraazacyclododecane-1,4,7,10 tetraacetic acid (DOTA), DOTA-GA (α-(2-carboxymethyl)-1,4,7,10-tetraazacyclododecane-1,4,7,10-tetraacetic acid), 1,4,7-triazacyclononane-1,4,7-triacetic acid (NOTA), ethylenediaminetetraacetic acid (EDTA), ethylenediaminediacetic acid, triethylenetetramine hexaacetic acid (TTHA), 1,4,8,11-tetraazacyclotetradecane-1,4,8,11-tetraacetic acid (TETA), dipyridoxyldiphosphate (DPDP), TPPS4, ethylenebishydroxyphenylglycine (EHPG), hexamethylenediaminetetetraacetic acid, dimethylphosphinomethane (DMPE), methylene diphosphate, dimercaptosuccinic acid (DMPA), and derivatives thereof.

Preferred combinations of the metal radionuclide and the chelating agent can be selected as appropriate by those skilled in the art (see, for example, Hiroshi SAKURAI and Yo Yokoyama, Introduction to Radiology), and examples thereof include ¹¹¹In and DTPA; ⁸⁹Zr and deferoxamine; ⁶⁴Cu and DOTA are NOTA; ^{99m}Tc with dimethylphosphinomethane (DMPE), DTPA, methylene diphosphate, dimercaptosuccinic acid (DMPA), dithiosemicarbazone, or diaminoethanediol; ^{67/68}Ga and deferoxamine or a DTPA derivative, and the like, preferably ¹¹¹In and DTPA; ⁸⁹Zr and deferoxamine; and ⁶⁴Cu and DOTA are NOTA, more preferably ¹¹¹In and DTPA; and ⁸⁹Zr and deferoxamine, more preferably ¹¹¹In and DTPA, still more preferably ⁸⁹Zr and DOTAs (for example, DOTA or DOTA-GA), and ²²⁵Ac and DOTA (for example, DOTAs or DOTA-GA).

The chelating agent other than those exemplified above is not particularly limited as long as it has a site to which the metal radionuclide is coordinated in the structure, and examples thereof include CB-TE2A (1,4,8,11-tetraazabicyclo[6.6.2]hexadecane-4,11-diacetic acid), CDTA (cyclohexane-trans-1,2-diaminetetraacetic acid), CDTPA (4-cyano-4-[[(dodecylthio)thioxomethyl]thio]-pentanoic acid), DOTMA ((1R,4R,7R,10R)-α,α′,α″,α′″-tetramethyl-1,4,7,10-tetraazacyclododecane-1,4,7,10-tetraacetic acid), DOTAM (1,4,7,10-tetrakis(carbamoylmethyl)-1,4,7,10-tetraazacyclododecane), DOTP (((1,4,7,10-tetraazacyclododecane-1,4,7,10-tetrayl)tetrakis(methylene))tetraphosphonic acid), DOTMP (1,4,7,10-tetraazacyclododecane-1,4,7,10-tetrakis(methylenephosphonic acid)), DOTA-4AMP (1,4,7,10-tetraazacyclododecane-1,4,7,10-tetrakis(acetamidomethylenephosphonic acid), D02P (tetraazacyclododecane dimethanephosphonic acid), deferoxamine (DFO), DTPA-BMA (5,8-bis(carboxymethyl)-11-[2-(methylamino)-2-oxoethyl]-3-oxo-2,5,8,11-tetraazatridecane-13-acid), NOTP (1,4,7-triazacyclononane-1,4,7-tolyltris(methylenephosphonic acid), TETPA (1,4,8,11-tetraazaacyclotetradecane-1,4,8,11-tetrapropionic acid), HEHA (1,2,7,10,13-hexaazacyclooctadecane-1,4,7,10,13,16-hexaacetic acid), 1,2-HOPO (N,N′,N″,N′″-tetra(1,2-dihydro-1-hydroxy-2-oxopyridine-6-carbonyl)-1,5,10,14-tetraazatetradecane), PEPA (1,4,7,10,13-pentaazacyclopentadecane-N,N′,N″,N′″,N″″-pentaacetic acid), H4octapa (N,N′-bis(6-carboxy-2-pyridylmethyl)-ethylenediamine-N,N′-diacetic acid), H2bispa2 (6,6′-({9-hydroxy-1,5-bis(methoxycarbonyl)-2,4-di(pyridin-2-yl)-3,7-diazabicyclo[3.3.1]nonane-3,7-diyl}bis(-methylene))dipicolinic acid), H2dedpa (1,2-[{6-(carboxy)-pyridin-2-yl}-methylamino]ethane), H2macropa (6-(1,4,10,13-tetraoxa-7,16-diazacyclooctadecane-N,N′-petyl)picolinic acid), H5decapa (N,N″-bis(6-carboxy-2-pyridylmethyl)-diethylenetriamine-N,N′,N″-triacetic acid), H6phospa (N,N′-(methylenephosphonate)-N,N′-[6-(methoxycarbonyl)pyridin-2-yl]-methyl-1,2-diaminoethane), HP-D03A (hydroxypropyltetraazacyclododecanetriacetic acid) or porphyrin. The chelate site preferably includes a structure represented by any one of Formulae (A) to (K) below (hereinafter, it is also referred to as a “chelating moiety”). These structures can be appropriately selected according to the type of the metal radionuclide described later. In certain preferred aspects, the chelate site includes a chelating moiety represented by Formula (A) below, and more preferably, the chelate site may be DOTAs, particularly DOTA or a derivative of DOTA-GA.

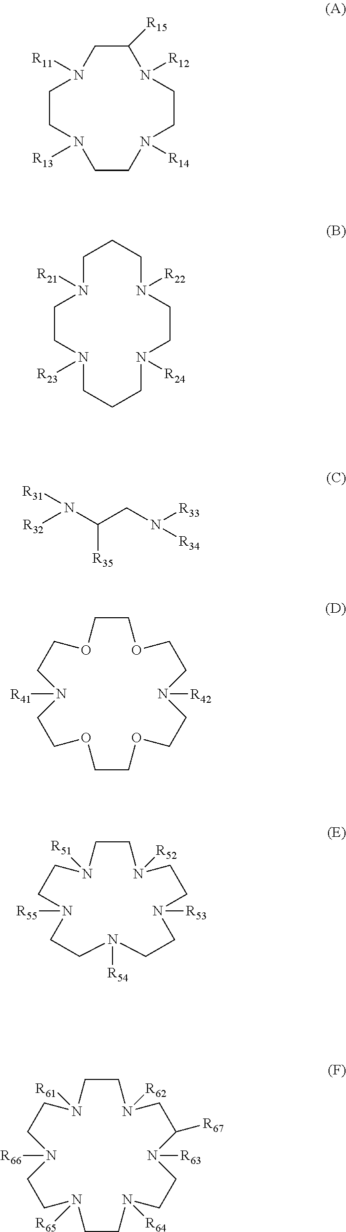

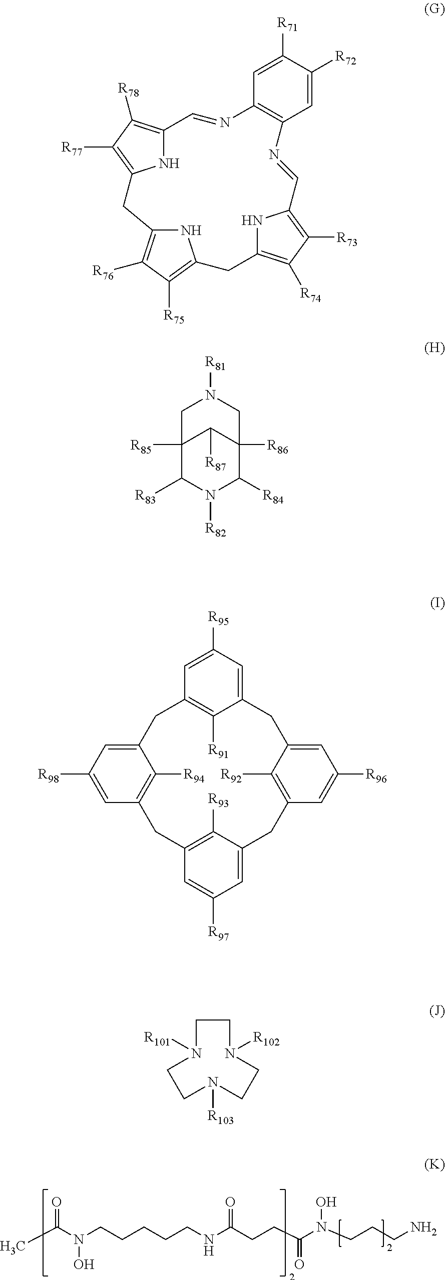

In Formula (A), R₁₁, R₁₃, and R₁₄ are each independently a group consisting of —(CH₂)ₚCOOH, —(CH₂)ₚC₅H₅N, —(CH₂)ₚPO₃H₂, —(CH₂)ₚCONH₂, or —(CHCOOH)(CH₂)ₚCOOH, one of R₁₂ and R₁₅ is a hydrogen atom, a carboxyl group, or a carboxyalkyl group having 2 or 3 carbon atoms, the other is a group bonded to a modification site bonded to a first atomic group described later, and p is an integer of 0 or more and 3 or less.

In Formula (B), R₂₁, R₂₂, R₂₃, and R₂₄ each independently represent a carboxyl group or a carboxyalkyl group having 2 or 3 carbon atoms, and any one group of R₂₁, R₂₂, R₂₃, or R₂₄ is a group bonded to the above modification site.

In Formula (C), R₃₁, R₃₂, R₃₃, and R₃₄ are each independently a group having a hydrogen atom and 2 or more and 10 or less carbon atoms and composed of an atomic group which may contain a nitrogen atom or an oxygen atom, and R₃₅ is a group bonded to the above modification site.

In Formula (D), one of R₄₁ and R₄₂ is a group having a hydrogen atom and 5 or more and 20 or less carbon atoms and composed of an atomic group containing one or more selected from a nitrogen atom, an oxygen atom and a sulfur atom, and the other is a group bonded to the above modification site.

In Formula (E), R₅₁, R₅₂, R₅₃, R₅₄, and R₅₅ are each independently a carboxyl group or a carboxyalkyl group having 2 or 3 carbon atoms, provided that any one group of R₅₁, R₅₂, R₅₃, R₅₄, or R₅₅ is a group bonded to the above modification site.

In Formula (F), R₆₁, R₆₂, R₆₃, R₆₄, R₆₅, and R₆₆ each independently represent a carboxyl group or a carboxyalkyl group having 2 or 3 carbon atoms, and R₆₇ represents a group bonded to the above modification site.

In Formula (G), R₇₁ and R₇₂ are —O(CH₂CH₂O)ₙCH₃ (here, n is an integer of 1 or more and 5 or less), R₇₃, R₇₅, R₇₆ and R₇₈ are each independently an alkyl group having 1 or more and 5 or less carbon atoms, and R₇₄ or R₇₇ is a group in which one of them is a hydroxyalkyl group having 1 or more and 5 or less carbon atoms, and the other is a group bonded to the above modification site.

In Formula (H), R₈₁ and R₈₂ are each independently an alkyl group having 1 to 5 carbon atoms, the terminal of the alkyl group may be substituted with a pyridyl group substituted with 1 or more carboxyl groups, R₈₇ is —CHOH or —C═O, but one group of R₈₁, R₈₂ or R₈₇ is a group bonded to the above modification site, R₈₃ and R₈₄ are an optionally substituted pyridinyl group, R₈₅ and R₈₆ are each independently —COORa, and Ra is an alkyl group having 1 to 5 carbon atoms.

In Formula (I), R₉₁, R₉₂, R₉₃, and R₉₄ are each independently —OCH₂COOH, one group of R₉₁, R₉₂, R₉₃, or R₉₄ is a group bonded to the above modification site, and R₉₅, R₉₆, R₉₇, and R₉₈ are each independently an alkyl group having 1 to 6 carbon atoms.

In Formula (J), R₁₀₁, R₁₀₂, and R₁₀₃ are each independently a carboxyl group or a carboxyalkyl group having 2 or 3 carbon atoms, or in Formula (J), at least one of R₁₀₁, R₁₀₂, and R₁₀₃ is a group bonded to the above modification site, and the other group is a carboxyl group or a carboxyalkyl group having 2 or 3 carbon atoms.

In Formulae (A) to (J) above, the “group bonded to the modification site” refers to a structure derived from a carboxyl group, an amino group, an N-hydroxysuccinimide ester (NHS) group, a 2,6-dioxotetrahydro-2H-pyranyl group, an isocyanate group, or an isothiocyanate group, and a structure in which the modification site is bonded.

Specific examples of the structure represented by Formula (A) include structures derived from compounds represented by Formulae (A-1) to (A-12) below.

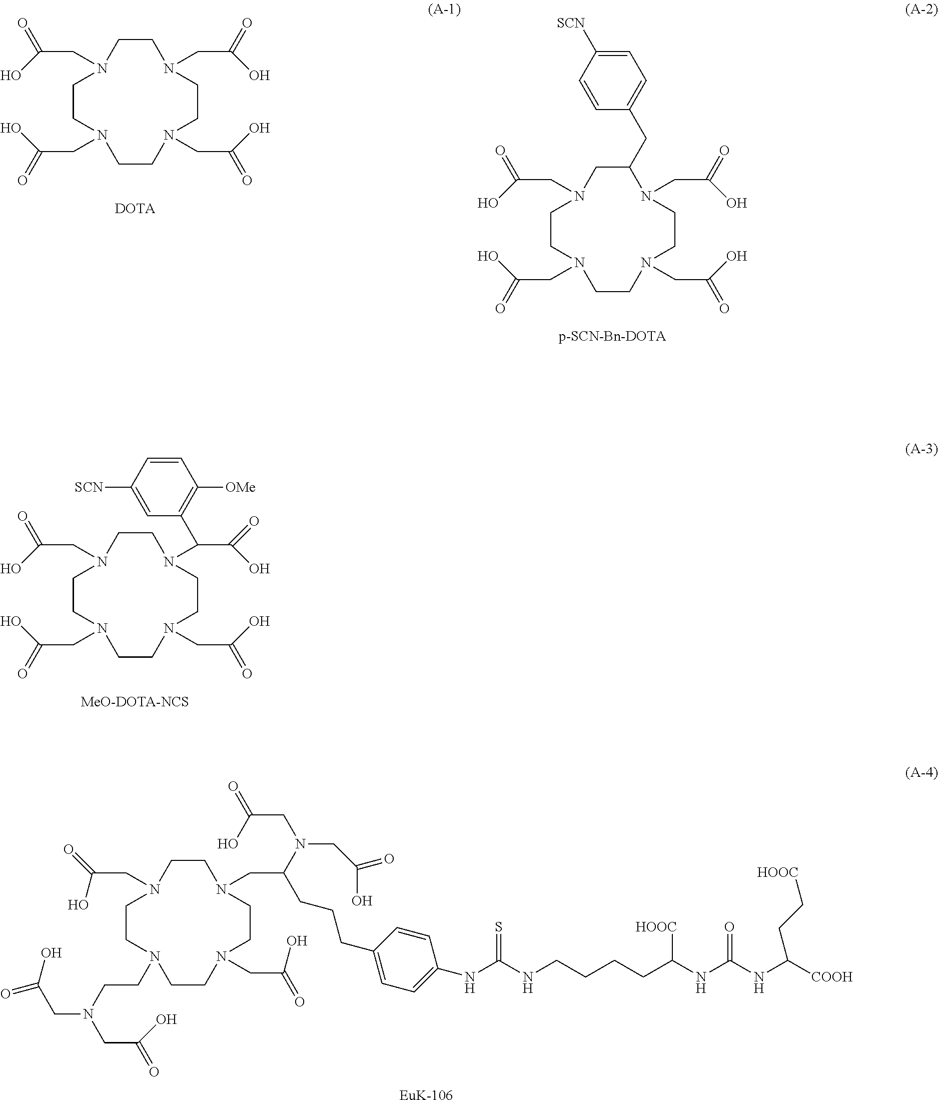

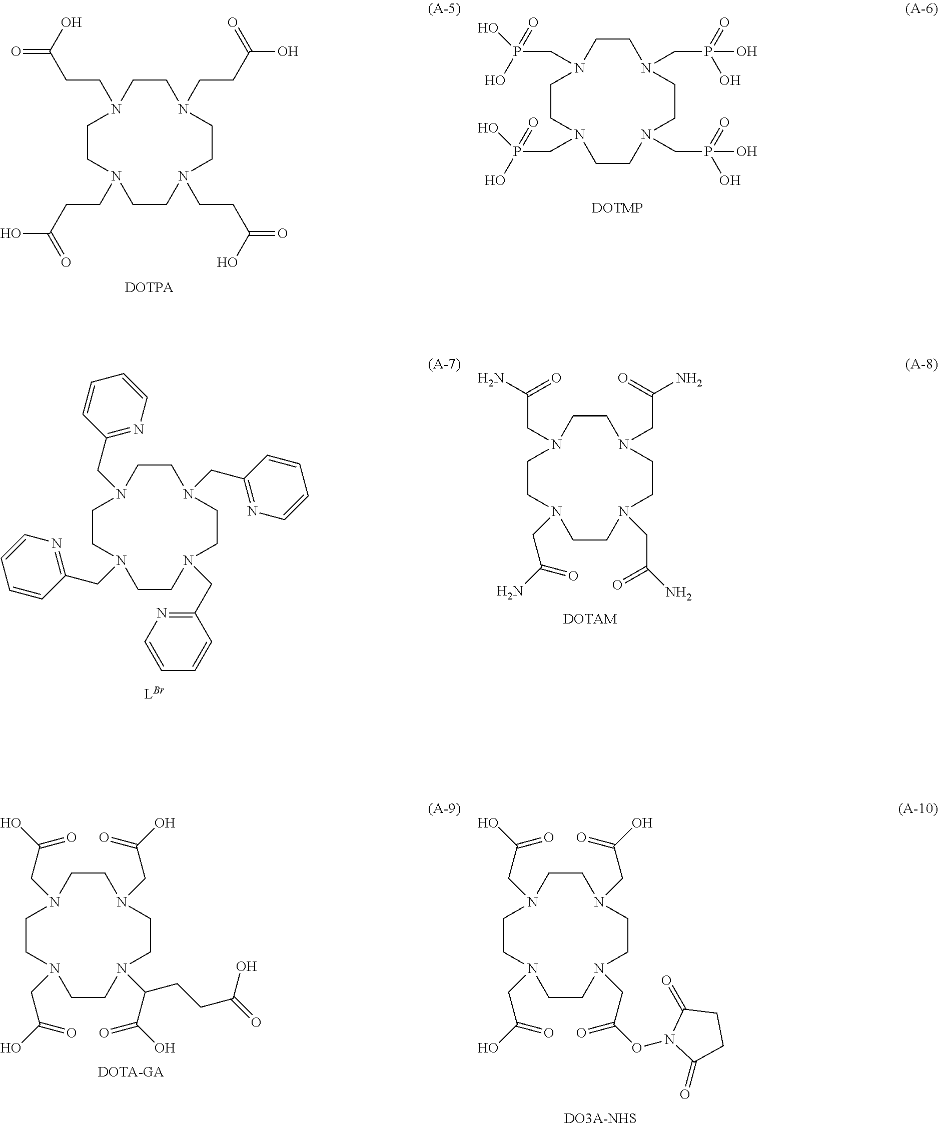

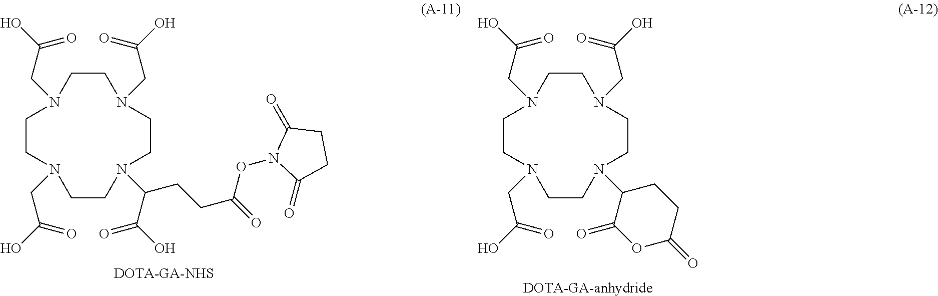

Specific examples of the structure represented by Formulae (B) and (C) include structures derived from compounds represented by Formulae (B-1), (B-2), and (C-1) to (C-5) below.

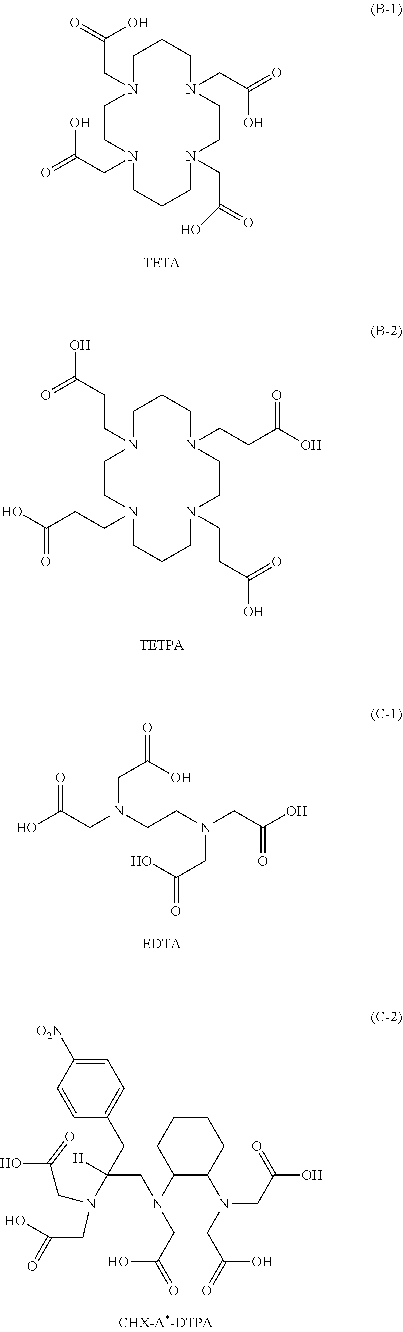 [SMILES: O=C(O)CN1CCCN(CC(=O)O)CCN(CC(=O)O)CCCN(CC(=O)O)CC1 ; O=C(O)CCN1CCCN(CCC(=O)O)CCN(CCC(=O)O)CCCN(CCC(=O)O)CC1 ; O=C(O)CN(CCN(CC(=O)O)CC(=O)O)CC(=O)O ; O=C(O)CN(CC(=O)O)C(Cc1ccc([N+](=O)[O-])cc1)CN(CC(=O)O)C1CCCCC1N(CC(=O)O)CC(=O)O]

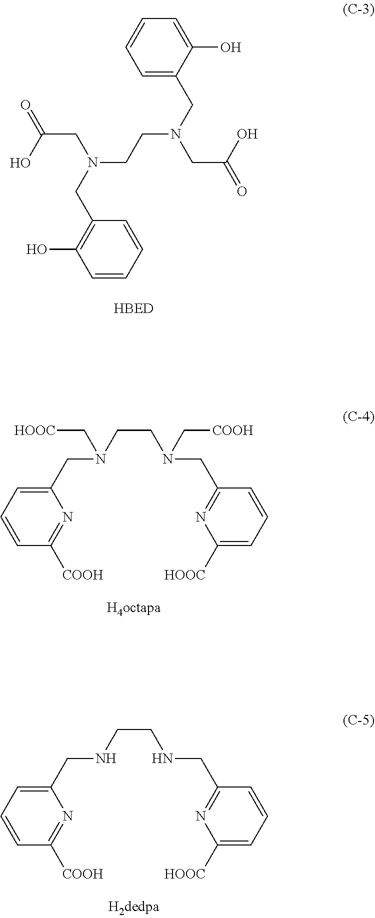 [SMILES: O=C(O)CN(CCN(CC(=O)O)Cc1ccccc1O)Cc1ccccc1O ; O=C(O)CN(CCN(CC(=O)O)Cc1cccc(C(=O)O)n1)Cc1cccc(C(=O)O)n1 ; O=C(O)c1cccc(CNCCNCc2cccc(C(=O)O)n2)n1]

Specific structures represented by Formulae (D) and (E) include structures derived from compounds represented by Formulae (D-1) to (D-3) and (E-1) below.

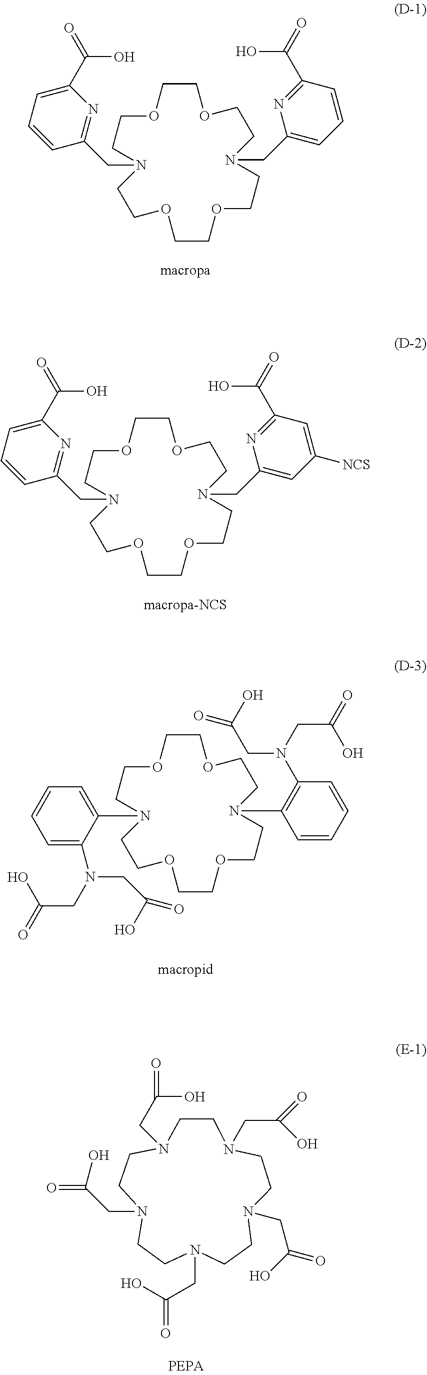

Specific structures represented by Formulae (F) and (G) include structures derived from compounds represented by Formulae (F-1) to (F-2) and (G-1) below.

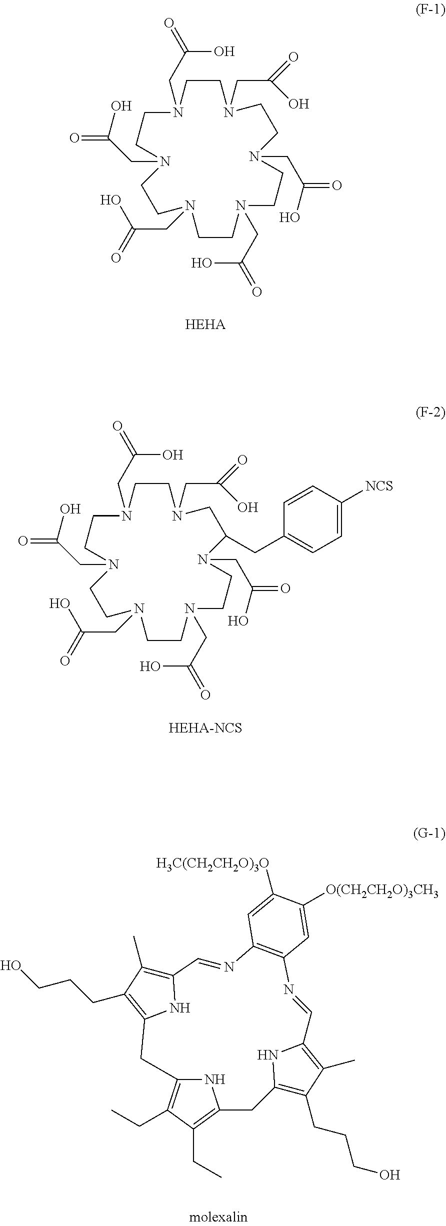

Specific structures represented by Formulae (H) and (I) include structures derived from compounds represented by Formulae (H-1) to (H-4) and (I-1) below.

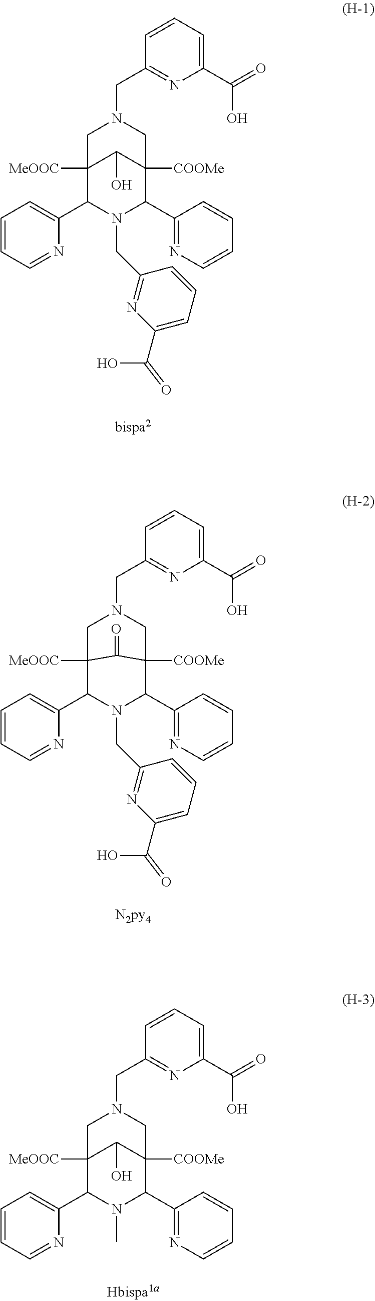 [SMILES: COC(=O)C12CN(Cc3cccc(C(=O)O)n3)CC(C(=O)OC)(C(c3ccccn3)N(Cc3cccc(C(=O)O)n3)C1c1ccccn1)C2O ; COC(=O)C12CN(Cc3cccc(C(=O)O)n3)CC(C(=O)OC)(C1=O)C(c1ccccn1)N(Cc1cccc(C(=O)O)n1)C2c1ccccn1 ; COC(=O)C12CN(Cc3cccc(C(=O)O)n3)CC(C(=O)OC)(C(c3ccccn3)N(C)C1c1ccccn1)C2O]

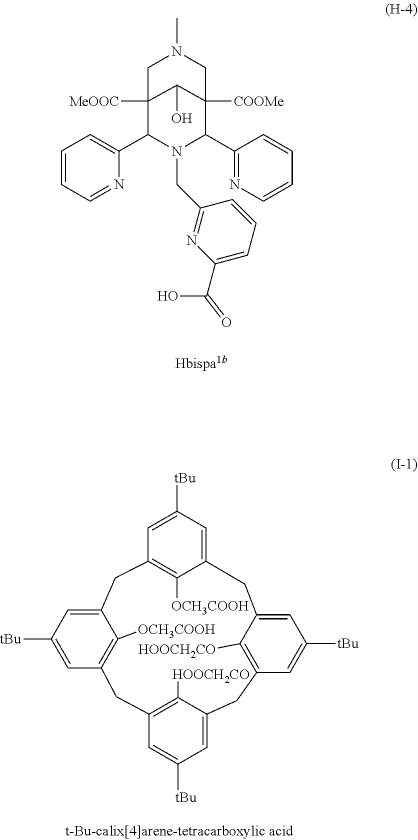

Specific examples of the structure represented by Formula (J) include structures derived from compounds represented by Formulae (J-1) to (J-5) below.

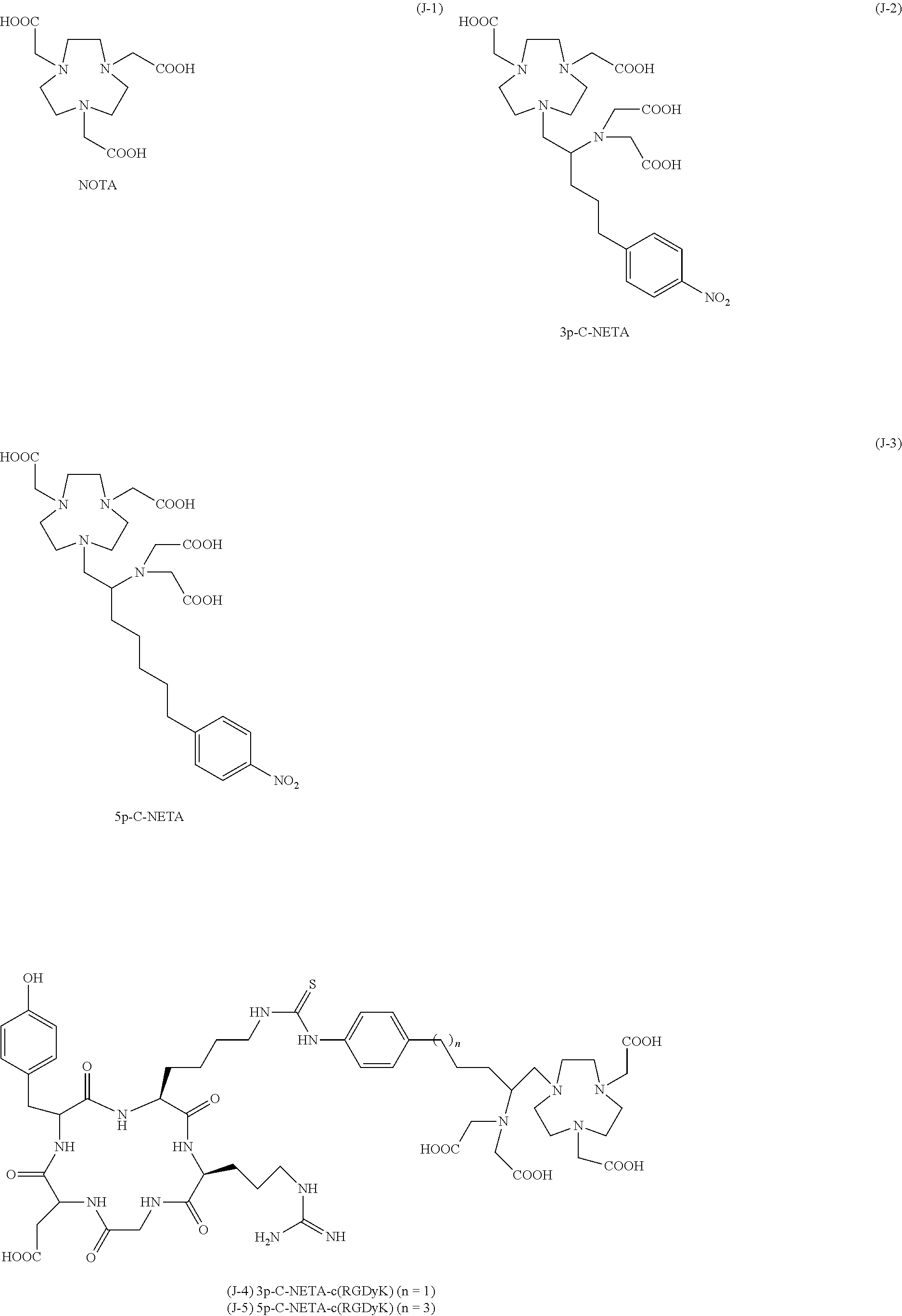

The binding site between the chelating moiety and the modification site is preferably an amide bond or a thiourea bond as described above, but is more preferably an amide bond from the viewpoint of further increasing the yield.

The amide bond is formed, for example, by reaction of a carboxyl group of a compound represented by any one of Formulae (B-1), (B-2), (G-1), (H-1) to (H-4), (I-1), (J-1) to (J-3) above, an N-hydroxysuccinimide ester (NHS) group of any one of Formulae (A-10) and (A-11), or a 2,6-dioxotetrahydro-2H-pyranyl group of Formula (A-12) above with a primary amine, or is formed by reaction of an amino group described at the right end in the drawing of a compound represented by Formula (K) with a reagent having a hydroxy group, a carboxyl group, or an NHS group. Here, in the case of reacting with a reagent having a hydroxy group, the hydroxy group is converted into a carboxyl group and used. In addition, the thiourea bond is formed by the reaction between the isothiocyanate group of the compound represented by Formula (A-2), (A-3), (D-2), or (F-2) above and the primary amine or the maleimide group.

The modification site can be formed by variously selecting from commercially available reagents having a desired click-reactive first atomic group bonded thereto and including a primary amine or commercially available reagents capable of forming an amide bond or a thiourea bond.

As the combination of the atomic groups capable of click reaction, an appropriate combination is selected according to the type of click reaction, and examples thereof include a combination of alkyl and azide, a combination of 1,2,4,5-tetrazine and alkene, and the like. These atomic groups may include an atomic group in which the first atomic group has one of the atomic groups and the second atomic group introduced into the antibody is a combination of the first atomic groups. It is preferable that the first atomic group is alkyne and the second atomic group is azide, or the first atomic group is 1,2,4,5-tetrazine and the second atomic group is alkene from the viewpoint of achieving both the complex stability of the metal radionuclide and the stability of the antibody and the improvement of the binding efficiency thereof. Specific examples of the click reaction by such a combination of atomic groups include a Huisgen cycloaddition reaction and a reverse electron request Diels-Alder reaction.

Specific examples of the combination of the click-reactive atomic groups include a combination of an atomic group containing dibenzylclooctyne (DBCO) as the alkyne of the first atomic group and an atomic group containing an azide group as the azide of the second atomic group, or a combination of an atomic group containing 1,2,4,5-tetrazine as the first atomic group and an atomic group containing trans-cyclooctene (TCO) as the alkene of the second atomic group.

As such a reagent, in a case where DBCO is used as the first atomic group, DBCO-amine, DBCO-maleimide, DBCO-PEG-NHS ester, DBCO-PEG-alcohol, DBCO-PEG-amine, DBCO-PEG-maleimide, and the like can be selected, and DBCO-amine, DBCO-maleimide, DBCO-PEG-amine, DBCO-PEG-maleimide can be preferably selected.

The introduction of the second atomic group into the antibody will be described later.

In one aspect of the present invention, the chelate site can be formed by reacting a ligand having a structure in which an appropriate one is selected from among the first atomic group, the chelating moiety, and the modification site described above with the metal radionuclide, and preferably, can be formed by reacting a ligand having a structure represented by Formula (ii) below with the metal radionuclide.

A-B-C (ii)

In Formula (ii), A is represented by Formula (iia) below.

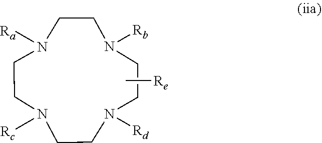

In Formula (iia), Rₐ, R_{b}, and R_{c} are independently a group consisting of —(CH₂)ₚCOOH, —(CH₂)ₚC₅H₅N, —(CH₂)ₚPO₃H₂, —(CH₂)ₚCONH₂, or —(CHCOOH)(CH₂)ₚCOOH, p is an integer of 0 or more and 3 or less, one of R_{d} and Rₑ is a binding site (*) with B, the other is a hydrogen atom, or a group consisting of —(CH₂)ₚCOOH, —(CH₂)ₚC₅H₅N, —(CH₂)ₚPO₃H₂, —(CH₂)ₚCONH₂, or —(CHCOOH)(CH₂)ₚCOOH, and p is an integer of 0 or more and 3 or less.

In Formula (ii), B is represented by Formula (iib) below.

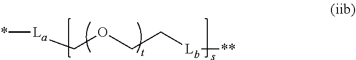

In Formula (iib), Lₐ and L_{b} are independently a binding linker having 1 or more and 50 or less carbon atoms and containing at least an amide bond or a thiourea bond, t is an integer of 0 or more and 30 or less, s is 0 or 1, * is a binding site with A, and ** is a binding site with C.

In Formula (ii), C represents an alkyne derivative represented by Formula (iic) below or a tetrazine derivative represented by Formula (iid) below.

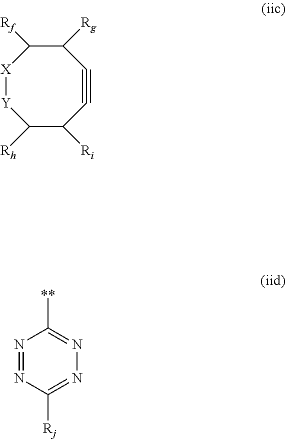

In Formula (iic), X is CHRₖ—** or N—**, Y is CHRₖ or C═O, Rₖ is independently a hydrogen atom or an alkyl group having 1 or more and 5 or less carbon atoms, and in a case where X is CHRₖ—** and Y is CHRₖ, the Rₖ moiety may form a cycloalkyl group together, R_{f}, R_{g}, Rₕ, and Rᵢ are independently a hydrogen atom, a halogen atom, an alkyl group having 1 or more and 5 or less carbon atoms, R_{f} and R_{g} may form a hydrocarbon ring together, or Rₕ and Rᵢ may form a hydrocarbon ring together, ** represents a binding site with B, and in Formula (iid), ** represents a binding site with B, and Rⱼ represents a hydrogen atom, a methyl group, a phenyl group, or a pyridyl group.

In addition, more preferably, the ligand, as A, is a DOTA derivative in Formula (iia) above wherein Rₐ to R_{d} are —(CH₂)ₚCOOH, p is 1, and Rₑ is the binding site with B; or either a DO3A derivative or a DOTAGA derivative wherein Rₐ to R_{c} are —(CH₂)ₚCOOH, p is 1, R_{d} is a binding site with B (*), and Rₑ is a hydrogen atom.

In Formula (ii), a DOTA-PEGt-DBCO derivative in which, in a case where A is the above-mentioned DOTA derivative, in B, Lₐ is a binding linker containing a thiourea bond and having not less than 1 and not more than 50 carbon atoms, s is 0 or 1, in a case where s is 1, t is an integer of not less than 0 and not more than 30, L_{b} is a binding linker containing an amide bond or a thiourea bond and having not less than 1 and not more than 50 carbon atoms, and C is an alkyne derivative represented by Formula (iic), in which, in Formula (iic), X is N—**, Y is CHRₖ, Rₖ is a hydrogen atom, R_{f} and R_{g} are bonded to form a benzene ring, Rₕ and Rᵢ are bonded to form a benzene ring, and is a binding site with B; or a DOTA-PEGt-Tz derivative in which, in B, Lₐ is a binding linker containing a thiourea bond and having not less than 1 and not more than 50 carbon atoms, s is 0 or 1, in a case where s is 1, t is an integer of not less than 0 and not more than 30, L_{b} is a binding linker containing an amide bond or a thiourea bond and having not less than 1 and not more than 50 carbon atoms, and C is a tetrazine derivative represented by Formula (iid), is further more preferred.

In Formula (ii), a DO3A-PEGt-DBCO derivative in which in a case where A is the above DO3A derivative, in B, Lₐ is a binding linker containing an amide bond or a thiourea bond and having not less than 1 and not more than 50 carbon atoms, s is 0 or 1, in a case where s is 1, t is an integer of not less than 0 and not more than 30, L_{b} is a binding linker containing an amide bond or a thiourea bond and having not less than 1 and not more than 50 carbon atoms, and C is an alkyne derivative represented by Formula (iic), in which, in Formula (iic), X is N—**, Y is CHRₖ, Rₖ is a hydrogen atom, R_{f} and R_{g} are jointed to form a benzene ring, Rₕ and Rᵢ are bonded to form a benzene ring, and is a binding site with B is further more preferred.

In Formula (ii), a DOTAGA-PEGt-DBCO derivative in which in a case where A is the above DOTAGA derivative, in B, Lₐ is a binding linker containing an amide bond or a thiourea bond and having not less than 1 and not more than 50 carbon atoms, s is 0 or 1, when s is 1, t is an integer of not less than 0 and not more than 30, L_{b} is a binding linker containing an amide bond or a thiourea bond and having not less than 1 and not more than 50 carbon atoms, and C is an alkyne derivative represented by Formula (iic), in which, in Formula (iic), X is N—**, Y is CHRₖ, Rₖ is a hydrogen atom, R_{f} and R_{g} are jointed to form a benzene ring, Rₕ and Rᵢ are bonded to form a benzene ring, and is a binding site with B is further more preferred.

In the antibody-RI conjugate of the present disclosure, it is sufficient that at least one or more chelate sites are provided for one antibody, but it is preferable that 1 or more and 8 or less chelate sites are provided. However, from the viewpoint of maintaining the activity (antigen recognition, neutralization, complement activation, and/or opsonic action) of the antibody itself, it is preferable that a chelate site is site-specifically introduced into the Fc region (constant region) of the antibody, and in the antibody-RI conjugate of the present disclosure, it is more preferable that one or two chelate sites are provided for one antibody.

### (4) Antibody Modification Linker

According to the present disclosure, a linker can be linked to an amino acid residue of an antibody. In certain aspects, the linker may be linked to an amino group of the antibody. In certain aspects, the linker may be linked to a cysteine group of the antibody. In certain aspects, the antibody is an IgG antibody and the linker may be linked via an IgG-binding peptide that binds to the Fc region of the IgG antibody (see, for example, WO2016/186206, WO2017/217347, and WO2021/075546). In this method, an antibody and an IgG-binding peptide can be bound based on affinity to form an antibody-peptide complex in a stoichiometric ratio of 1:1 or 1:2 between the antibody and the IgG-binding peptide. The reason why the stoichiometric ratio between the antibody and the peptide is 1:1 or 1:2 is that there is a site where the peptide binds to each heavy chain constant region one by one. By adjusting the reaction between the antibody and the peptide, a complex having a stoichiometric ratio of 1:1 or a complex having a stoichiometric ratio of 1:2 can be preferentially obtained. The antibody and the peptide are non-covalent linkage to form a complex due to the binding affinity of the peptide to the antibody, but the antibody and the peptide can be crosslinked by a crosslinking agent, and in this way, a more stable complex in which the antibody and the peptide are covalently linked can be obtained. The chelate site that chelates the metal radionuclide can be covalently linked to the peptide. In this way, a complex comprising the antibody and the chelate site (that is, the metal radionuclide) in a stoichiometric ratio of 1:1 or 1:2 can be formed.

As the IgG-binding peptide, the following peptides disclosed in WO2017/217347 can be used. In certain aspects, the peptide contains an amino acid sequence consisting of 13 to 17 amino acid residues, represented by Formula I below:

(X₁₋₃)-C-(X₂)—H-(Xaa1)-G-(Xaa2)-L-V-W-C-(X₁₋₃) (I)
(wherein each of X is independently any amino acid residue other than cysteine,
C is a cysteine residue,
H is a histidine residue,
Xaa1 is a lysine residue, a cysteine residue, an aspartic acid residue, a glutamic acid residue, 2-aminosuberic acid, or diaminopropionic acid,
G is a glycine residue,
Xaa2 is a glutamic acid residue, a glutamine residue, or an asparagine residue,
L is a leucine residue,
V is a valine residue, and
W is a tryptophan residue)
and is capable of binding human IgG.

In the above formula, the notation X₁₋₃ at the N-terminal side or the C-terminal side means that one to three independently arbitrary amino acid residues X other than cysteine (C or Cys) are consecutive, and the amino acid residues constituting it are the same or different residues, but preferably all three amino acid residues X consist of a sequence that is not the same residue. Similarly, X₂ also means that two independently arbitrary amino acid residues X other than cysteine (C or Cys) are consecutive, and the amino acid residues constituting X₂ are the same or different residues, but preferably the two consecutive amino acid residues consist of a sequence that is not the same residues.

The two cysteine residues of Formula I can be disulfide bonded to form a cyclic peptide. Typically, in the peptide of Formula I, the two outer cysteine residues (not Xaa1 if Xaa1 is a cysteine residue) are disulfide-bonded. Alternatively, in the peptide of Formula I, the sulfide groups in the two outer cysteine residues may be linked by a linker represented by the following formula:

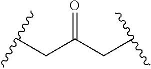 [SMILES: *CC(=O)C*]

The broken line part in the above formula means a binding portion with a sulfide group. The linker is more stable to a reduction reaction and the like than a normal disulfide bond. Therefore, the linker is preferably used when the metal radionuclide capable of destabilizing a disulfide bond such as zirconium is used.

This peptide can be obtained, for example, by the following method:
a method including a process of mixing a peptide containing two or more, preferably two, cysteine residues with a compound (wherein, R₁ and R₂ are each independently any halogen atom) represented by the following formula:

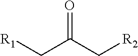

and obtaining a peptide in which two or more, preferably two, sulfide groups in cysteine residues are linked by a linker represented by the following formula:

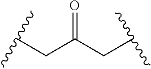 [SMILES: *CC(=O)C*]

The broken line part in the above formula means a binding portion with a sulfide group.

In the compounds, R₁ and R₂ are preferably selected from the group consisting of F, Cl, Br and I, more preferably Cl, Br and I. R₁ and R₂ are preferably the same, and more preferably, both R₁ and R₂ are Cl.

The peptides represented by Formula I′ and Formula I″ in which the amino acid residue X is further specified in the amino acid sequence of the peptide of Formula I are shown below.

That is, the peptide represented by Formula I′ contains an amino acid sequence consisting of 13 to 17 amino acid residues, represented by

(X₁₋₃)-C-(X₁)—Y—H-(Xaa1)-G-N-L-V-W-C-(X₁₋₃) (I′)
(wherein each X is independently any amino acid residue other than cysteine, and
Xaa1 is a lysine residue, cysteine residue, aspartic acid residue, glutamic acid residue, 2-aminosuberic acid, or diaminopropionic acid).

The peptide represented by Formula I″ contains an amino acid sequence consisting of 13 to 17 amino acid residues, represented by

(X₁₋₃)-C-A-(X₁)—H-(Xaa1)-G-E-L-V-W-C-(X₁₋₃) (I″)
(wherein each X is independently any amino acid residue other than cysteine, and
Xaa1 is a lysine residue, cysteine residue, aspartic acid residue, glutamic acid residue, 2-aminosuberic acid, or diaminopropionic acid).

In addition, the peptides represented by Formula II in which the amino acid residue X is further specified in the amino acid sequence of the peptide of Formula I are shown below.

That is, the peptide represented by Formula II contains an amino acid sequence consisting of 13 to 17 amino acid residues, represented by

(X₁₋₃)-C-(Xaa3)-(Xaa4)-H-(Xaa1)-G-(Xaa2)-L-V-W-C-(X₁₋₃) (II)
(wherein each of X is independently any amino acid residue other than cysteine,

Xaa1 is a lysine residue, a cysteine residue, an aspartic acid residue, a glutamic acid residue, 2-aminosuberic acid, or diaminopropionic acid,
Xaa2 is a glutamic acid residue, a glutamine residue, or an asparagine residue,
Xaa3 is an alanine residue, a serine residue, or a threonine residue, and
Xaa4 is a tyrosine residue or a tryptophan residue).

In the amino acid sequences of the peptides of Formula I′, Formula I″, and Formula II described above, in the case of 17 amino acid residues, the first and second and sixteenth and seventeenth amino acid residues X from the N-terminal side may be deleted, and such a peptide is 13 amino acids long.

As used herein, the term “in the case of 17 amino acid residues” is a term expressed for convenience in order to number the first to seventeenth amino acid residues in order from the N-terminal side of 17 residues which is the longest amino acid length in the peptide of Formula I when the amino acid residues of the peptide are referred to by amino acid numbers.

In addition, the peptides represented by Formula III in which the amino acid residue X is further specified in the amino acid sequence of the peptide of Formula I are shown below.

That is, the peptide represented by Formula III contains an amino acid sequence consisting of 13 to 17 amino acid residues, represented by

(X₁₋₃)-C-A-Y—H-(Xaa1)-G-(Xaa2)-L-V-W-C-(X₁₋₃) (III)
(wherein each of X is independently any amino acid residue other than cysteine,
Xaa1 is a lysine residue, a cysteine residue, an aspartic acid residue, a glutamic acid residue, 2-aminosuberic acid, or diaminopropionic acid, and
Xaa2 is a glutamic acid residue or a glutamine residue).

In the amino acid sequence of the peptides of Formula III described above, in the case of 17 amino acid residues, the first and second and sixteenth and seventeenth amino acid residues X from the N-terminal side may be deleted, and such a peptide may be 13 amino acids long.

Furthermore, it is preferable that the amino acid residues other than cysteine (C) in the amino acid sequence of the peptide of each of the above formulae, that is, the 1 to 3, 5, 6, and 15 to 17-th amino acid residues from the N-terminal in the case of 17 amino acid residues are selected from the following. Here, each capital letter is a one-letter code for an amino acid:
the first amino acid residue=S, G, F, R or none;
the second amino acid residue=D, G, A, S, P, homocysteine, or none;
the third amino acid residue=S, D, T, N, E or R;
the fifteenth amino acid residue=S, T, or D;
the sixteenth amino acid residue=H, G, Y, T, N, D, F, homocysteine, or none; and
the seventeenth amino acid residue=Y, F, H, M, or none.

The fifth amino acid residue=A or T, and
the sixth amino acid residue=Y or W.

In addition, the peptides represented by Formula IV in which the amino acid residue X is further specified in the amino acid sequence of the peptide of Formula I are shown below.

That is, the peptide represented by Formula IV contains an amino acid sequence consisting of 13 amino acid residues, represented by

D-C-(Xaa3)-(Xaa4)-H-(Xaa1)-G-(Xaa2)-L-V-W-C-T (IV)
(wherein
Xaa1 is a lysine residue, a cysteine residue, an aspartic acid residue, a glutamic acid residue, 2-aminosuberic acid, or diaminopropionic acid,
Xaa2 is a glutamic acid residue, a glutamine residue, or an asparagine residue,
Xaa3 is an alanine residue or a threonine residue, and
Xaa4 is a tyrosine residue or a tryptophan residue).

Some of the specific examples of peptides of Formula I are listed in the following 1) to 18), but it goes without saying that it is not limited thereto:

1)
(SEQ ID NO: 185)
DCAYH (Xaa1) GELVWCT,
2)
(SEQ ID NO: 186)
GPDCAYH (Xaa1) GELVWCTFH,
3)
(SEQ ID NO: 187)
RCAYH (Xaa1) GELVWCS,
4)
(SEQ ID NO: 188)
GPRCAYH (Xaal) GELVWCSFH,
5)
(SEQ ID NO: 189)
SPDCAYH (Xaa1) GELVWCTFH,
6)
(SEQ ID NO: 190)
GDDCAYH (Xaa1) GELVWCTFH,
7)
(SEQ ID NO: 191)
GPSCAYH (Xaa1) GELVWCTFH,
8)
(SEQ ID NO: 192)
GPDCAYH (Xaa1) GELVWCSFH,
9)
(SEQ ID NO: 193)
GPDCAYH (Xaa1) GELVWCTHH,
10)
(SEQ ID NO: 194)
GPDCAYH (Xaa1) GELVWCTFY,
11)
(SEQ ID NO: 195)
SPDCAYH (Xaa1) GELVWCTFY,
12)
(SEQ ID NO: 196)
SDDCAYH (Xaa1) GELVWCTFY,
13)
(SEQ ID NO: 197)
RGNCAYH (Xaa1) GQLVWCTYH,
14)
(SEQ ID NO: 198)
G (Xaa2) DCAYH (Xaa1) GELVWCT (Xaa2) H
15)
(SEQ ID NO: 199)
DCTYH (Xaa1) GNLVWCT,
16)
(SEQ ID NO: 200)
DCAYH (Xaa1) GNLVWCT,
17)
(SEQ ID NO: 201)
DCTYH (Xaa1) GELVWCT,
and
18)
(SEQ ID NO: 202)
DCAWH (Xaa1) GELVWCT,

(wherein Xaa1 is a lysine residue, a cysteine residue, an aspartic acid residue, a glutamic acid residue, 2-aminosuberic acid, or diaminopropionic acid, Xaa2 is homocysteine, and preferably homocysteines form a disulfide bond with each other).

Preferred specific examples of the peptide of Formula I include

1)
(SEQ ID NO: 185)
DCAYH (Xaa1) GELVWCT,
2)
(SEQ ID NO: 186)
GPDCAYH (Xaa1) GELVWCTFH,
13)
(SEQ ID NO: 197)
RGNCAYH (Xaa1) GQLVWCTYH,
and
14)
(SEQ ID NO: 198)
G (Xaa2) DCAYH (Xaa1) GELVWCT (Xaa2) H
and

particularly preferred examples include 2) GPDCAYH (Xaa1) GELVWCTFH (SEQ ID NO: 186) (wherein Xaa1 is a lysine residue, Xaa2 is homocysteine, and preferably cysteines and/or homocysteines form a disulfide bond with each other).

In addition, in one aspect, the IgG-binding peptide of the present disclosure contains, as a broad primary structure, an amino acid sequence consisting of 13 amino acid residues, represented by Formula V below:

D-C-(Xaa2)-(Xaa3)-(Xaa4)-(Xaa1)-G-(Xaa5)-L-(Xaa6)-W—C-T (V)
(wherein
Xaa1 is a lysine residue, a cysteine residue, an aspartic acid residue, a glutamic acid residue, 2-aminosuberic acid, or diaminopropionic acid,
Xaa2 is an alanine residue, a serine residue, or a threonine residue,
Xaa3 is a tryptophan residue or a tyrosine residue,
Xaa4 is a histidine residue, an arginine residue, a serine residue, or a threonine residue,
Xaa5 is a glutamic acid residue, a glutamine residue, an asparagine residue, an arginine residue, or an aspartic acid residue, and
Xaa6 is an isoleucine residue or a valine residue).

The two cysteine residues of Formula V can be disulfide bonded to form a cyclic peptide. Typically, the two cysteine residues outside of the peptide of Formula V (not Xaa1 if Xaa1 is a cysteine residue) are disulfide-bonded. Alternatively, in the peptide of Formula V, the sulfide groups in the two outer cysteine residues may be linked by a linker represented by the following formula:

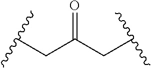 [SMILES: *CC(=O)C*]

The broken line part in the above formula means a binding portion with a sulfide group. The linker is more stable to a reduction reaction and the like than a normal disulfide bond. Therefore, the linker is preferably used when the metal radionuclide capable of destabilizing a disulfide bond such as zirconium is used. This peptide can be prepared by the method described in the present specification or in WO2017/217347.

Some of the specific examples of peptides of Formula V are listed in the following 18) to 29), but it goes without saying that it is not limited thereto:

18)
(SEQ ID NO: 203)
DCTYT (Xaa1) GNLVWCT,
19)
(SEQ ID NO: 204)
DCAYT (Xaa1) GNLVWCT,
20)
(SEQ ID NO: 205)
DCSYT (Xaa1) GNLVWCT,
21)
(SEQ ID NO: 206)
DCTWT (Xaa1) GNLVWCT,
22)
(SEQ ID NO: 207)
DCTYH (Xaa1) GNLVWCT,
23)
(SEQ ID NO: 208)
DCTYR (Xaa1) GNLVWCT,
24)
(SEQ ID NO: 209)
DCTYS (Xaa1) GNLVWCT,
25)
(SEQ ID NO: 210)
DCTYT (Xaa1) GNLVWCT,
26)
(SEQ ID NO: 211)
DCTYT (Xaa1) GELVWCT,
27)
(SEQ ID NO: 212)
DCTYT (Xaa1) GRLVWCT,
28)
(SEQ ID NO: 213)
DCTYT (Xaa1) GDLVWCT,
and
29)
(SEQ ID NO: 214)
DCTYT (Xaa1) GNLIWCT,

(wherein Xaa1 is a lysine residue, a cysteine residue, an aspartic acid residue, a glutamic acid residue, 2-aminosuberic acid, or diaminopropionic acid).

As described above, the peptide of the above formula according to the present disclosure is characterized by having at least two cysteine (C) residues separated from each other in each amino acid sequence, and disposing a cysteine residue so that a disulfide bond can be formed between the cysteine residues. In a preferred peptide, two cysteine residues are disulfide bonded to form a cyclic peptide, and one or two amino acid residues other than cysteine may be present on the N-terminal side and the C-terminal side of each cysteine residue. In the case of having one or two amino acid residues on the N-terminal side and the C-terminal side of each cysteine residue, the 1, 2, 16, and 17-th amino acid residues from the N-terminal in the case of 17 amino acid residues are those exemplified above.

As described above, in the IgG-binding peptide of the present disclosure, Xaa1 is a protein constituent amino acid such as a lysine residue, a cysteine residue, an aspartic acid residue, and a glutamic acid residue, and a non-proteinogenic amino acid such as diaminopropionic acid and 2-aminosuberic acid, preferably a lysine residue. It is preferable that Xaa1 can be modified with a crosslinking agent described later. In the present specification, the term “non-proteinogenic amino acid” refers to an amino acid that is not used to constitute a protein in a living body. In order to increase the site specificity when modifying the peptide of the present disclosure with a crosslinking agent, it is preferable that the IgG peptide of the present disclosure has no or almost no residue same as Xaa1 in its sequence (for example, there are only one or two). For example, in a case where Xaa1 is a lysine residue, the peptide of the present disclosure preferably has no or almost no lysine residue at a location other than Xaa1 in its sequence.

The IgG-binding peptide of the present disclosure has about 10 times or more, preferably about 50 times or more, more preferably about 200 times or more higher binding affinity with human IgG as compared to other human immunoglobulins (IgA, IgE, or IgM). The dissociation constant (Kd) for binding of a peptide of the present disclosure to the human IgG can be determined by surface plasmon resonance spectroscopy (for example, using the BIACORE system) and is, for example, from 1×10⁻¹ M to less than 1×10⁻³ M, preferably less than 1×10⁻⁴ M, more preferably less than 1×10⁻⁵ M.

The IgG-binding peptide of the present disclosure can be produced by a peptide synthesis method such as a conventional liquid phase synthesis method or solid phase synthesis method, peptide synthesis using an automatic peptide synthesizer, or the like (Kelley et al., Genetics Engineering Principles and Methods, Setlow, J. K. eds., Plenum Press NY. (1990) Vol. 12, p. 1-19; Stewart et al., Solid-Phase Peptide Synthesis (1989), W.H. Freeman Co.; Houghten, Proc. Natl. Acad. Sci. USA (1985) 82: p. 5132; “New Biochemistry Experiment Course 1, Protein IV” (1992), edited by The Biological Chemistry Society of Japan, Tokyo Kagaku Dojin Co., Ltd.). Alternatively, the peptide may be produced by a genetic recombination method using a nucleic acid encoding the peptide of the present disclosure, a phage display method, or the like. For example, a target peptide can be produced by incorporating a DNA encoding the amino acid sequence of the peptide of the present disclosure into an expression vector, introducing the DNA into a host cell, and culturing the host cell. The produced peptide can be recovered or purified by a conventional method, for example, chromatography such as gel filtration chromatography, ion exchange column chromatography, affinity chromatography, reverse phase column chromatography, or HPLC, ammonium sulfate fractionation, ultrafiltration, or an immunoadsorption method.

In peptide synthesis, for example, amino acids in which functional groups other than an α-amino group and an α-carboxyl group to be bonded of each amino acid (whether natural or non-natural) are protected are prepared, and a peptide bond forming reaction is performed between the α-amino group and the α-carboxyl group of each amino acid. Usually, the carboxyl group of the amino acid residue located at the C-terminal side of the peptide is bonded to the solid phase via an appropriate spacer or linker. The protecting group at the amino terminus of the dipeptide thus obtained is selectively removed to form a peptide bond with the α-carboxyl group of the next amino acid. Such an operation is continuously performed to produce a peptide in which the side group is protected, and finally, all the protecting groups are removed and separated from the solid phase. Details of the type of the protecting group, the protecting method, and the peptide bonding method are described in detail in the above-mentioned documents.

The IgG-binding peptide of the present disclosure may be modified by, for example, PEGylation at the N-terminal side (polyethylene glycol addition), amidation at the C-terminal side, or the like in order to improve the stability of the peptide synthesized by the above-described method. The number of molecules of PEG when PEGylation is performed is not particularly limited, and for example, 1 to 50 molecules, 1 to 20 molecules, 2 to 10 molecules, 2 to 6 molecules, or 4 molecules of PEG can be added.

In addition, the IgG-binding peptide in the present disclosure is preferably a peptide synthesized by the above-described method or a peptide having a PEGylated N-terminal side, into which a second atomic group capable of click-reacting with the first atomic group of the above-described ligand is introduced. Examples of the method for introducing the second atomic group into the IgG-binding peptide include a method in which a peptide having a desired amino acid sequence is obtained by the above-described method, then the peptide is dissolved in a solution to which a solubilizing agent, a reducing agent, and an acid as necessary are added, an organic solvent solution of an atomic group containing an azido group or a TCO is added as the second atomic group to the solution, and the mixture is stirred at room temperature to be introduced.

In a case where an atomic group containing the azide group is introduced as the second atomic group, an azide group can be introduced directly to the N-terminal or C-terminal of the peptide according to a conventional method using a commercially available azide group-introducing reagent, or an atomic group containing an azide group can be introduced via the above-described linker structure. Examples of the azide group-introducing reagent to be used include silyl azide, azide phosphate, alkylammonium azide, inorganic azide, sulfonyl azide, PEG azide, and the like.

In addition, in the case of introducing an atomic group containing TCO as the second atomic group, it is possible to introduce the TCO directly into the N-terminal or C-terminal of the peptide according to a conventional method using a commercially available click chemistry reagent containing the TCO, or to introduce an atomic group containing the TCO through the above-described linker structure.

In addition, the IgG-binding peptide of the present disclosure may be modified with a chelate site, a chelating agent, a chelating moiety, or a ligand by peptide synthesis using an amino acid residue modified with the chelate site, the chelating agent, the chelating moiety or the ligand described above.

The IgG-binding peptides of the present disclosure bind to a Fc domain of IgG. The IgG-binding peptides of the present disclosure are in proximity, in Xaa1 above, to a lysine residue in a specific region of IgG Fc, that is, the Fc region corresponding to Lys 248 residue (hereinafter, also referred to simply as “Lys 248” in the present specification, and corresponds to the twelfth residue of human IgG4 CH2 or human IgG4PE CH2 (SEQ ID NO: 220)) or Lys 246 (hereinafter, also simply referred to as “Lys 246” in the present specification, and corresponds to the sixteenth residue of human IgG1 CH2 (SEQ ID NO: 215) and corresponds to the tenth residue of human IgG4 CH2 or human IgG4PE CH2 (SEQ ID NO: 220)), preferably Lys 248, according to Eu numbering in human IgG Fc. Therefore, by modifying Xaa1 of the IgG-binding peptide of the present disclosure with a crosslinking agent and causing a cross-linking reaction with IgG, Xaa1 of the IgG-binding peptide can site-specifically form a crosslinked structure with a lysine residue of an IgG Fc of the present disclosure corresponding to the above lysine residue.

In the present disclosure, the “crosslinking agent” is a chemical substance for linking the IgG-binding peptide of the present disclosure and the IgG Fc by a covalent linkage. The crosslinking agent of the present disclosure can be appropriately selected by those skilled in the art, and can be a compound having at least two sites capable of binding to a desired amino acid (for example, a lysine residue, a cysteine residue, an aspartic acid residue, a glutamic acid residue, 2-aminosuberic acid, or diaminopropionic acid, arginine, or the like). Examples thereof include, but are not limited to, crosslinking agents preferably containing two or more succinimidyl groups, such as disuccinimidyl glutarate (DSG) and disuccinimidyl suberate (DSS), crosslinking agents preferably containing two or more imidic acid moieties, such as dimethyl adipimidate dihydrochloride (dimethyl adipimidate 2HCl, DMA), dimethyl pimelimidate dihydrochloride (dimethyl pimelimidate 2 HCl, DMP), and dimethyl suberimidate dihydrochloride (dimethyl suberimidate 2HCl, DMS), and crosslinking agents having an SS bond, such as 3,3′-dithiobispropionimidate dimethyl dihydrochloride (dimethyl 3,3′-dithiobispropionimidate 2HCl, DTBP) and dithiobis succinimidyl propionate (dithiobis(succinimidyl propionate), DSP).

The IgG-binding peptide modified with the crosslinking agent of the present disclosure can be produced, for example, by reacting the IgG-binding peptide obtained according to the method described above or the method described in the item <IgG-binding Peptide> in WO2017/217347 with a crosslinking agent. In this case, it is necessary to specifically modify the side chain of the amino acid residue of Xaa1 above in the IgG-binding peptide, and this can be performed, for example, by selecting a combination of the type of Xaa1 and the crosslinking agent. For example, since the crosslinking agent containing a succinimidyl group such as DSS or DSG reacts with a primary amine present on the side chain of a lysine residue and the N-terminal of the polypeptide, only the side chain of a lysine residue can be specifically modified with DSS or DSG by blocking the N-terminal of the IgG-binding peptide and then reacting with DSS or DSG. Such a combination of the amino acid residue and the crosslinking agent can be appropriately selected by those skilled in the art.

### (5) Linker Modified Antibody

In one aspect, the present disclosure relates to modified antibodies modified with a linker containing an IgG-binding peptide. The modified antibody can be formed by a cross-linking reaction with the IgG-binding peptide modified with the crosslinking agent. Thus, the present disclosure may preferably be a complex of the IgG-binding peptide and the IgG in which the amino acid residue of Xaa1 above of the IgG-binding peptide and a lysine residue of an Fc region of the IgG corresponding to Lys 248 or Lys 246, preferably Lys 248, according to Eu numbering are bound via a site-specific crosslinking agent. As described above, the Lys 28 corresponds to the eighteenth residue of the human IgG1 CH2 (SEQ ID NO: 215), and corresponds to the twelfth residue of human IgG4 CH2 or human IgG4PE CH2 (SEQ ID NO: 220). In addition, the Lys 246 corresponds to the eighteenth residue of the human IgG1 CH2 (SEQ ID NO: 215), and corresponds to the tenth residue of human IgG4 CH2 or human IgG4PE CH2 (SEQ ID NO: 220). In certain aspects, the antibody may be any of the humanized antibodies described above and/or the peptide can be any of the IgG-binding peptides described above.

In the peptide-modified antibody, one or two peptides are bound to the antibody by binding between the antibody and the IgG-binding peptide, and the antibody and the peptide are crosslinked by subsequent processing. In this way, an antibody-peptide complex in which an antibody and a peptide are covalently linked is provided.

In one aspect, the present disclosure relates to a method for producing a complex of the IgG-binding peptide and the antibody of the present disclosure containing mixing the antibody of the present disclosure with the IgG-binding peptide modified with a crosslinking agent of the present disclosure. By this process, the cross-linking reaction may occur between the IgG-binding peptide modified with the crosslinking agent and the antibody of the present disclosure. The cross-linking reaction may occur site-specifically, in particular between the amino acid residue of Xaa1 above of the IgG-binding peptide and a lysine residue of an IgG of the antibody of the present disclosure corresponding to Lys 18 or Lys 16, preferably Lys 18, in the human IgG1 constant region (CH) set forth in SEQ ID NO: 215.

The conditions for the mixing process are not particularly limited as long as the mixing process is performed under conditions where a cross-linking reaction occurs between the IgG-binding peptide of the present disclosure and the antibody of the present disclosure. For example, the reaction can be performed by mixing the IgG-binding peptide of the present disclosure and the antibody of the present disclosure in an appropriate buffer at room temperature (for example, about 15° C. to 30° C.). The mixing process may be performed by adding an appropriate amount of a catalyst that promotes the cross-linking reaction as necessary.

As an example, a solvent containing at least water is added to dissolve the antibody of the present disclosure. Examples of the solvent other than water include buffers such as dimethyl sulfoxide, acetonitrile, physiological saline, sodium acetate buffer, ammonium acetate buffer, phosphate buffer, phosphate buffer saline, Tris buffer, HEPES buffer, and tetramethylammonium acetate buffer. In a case where the buffer is used, the pH at 25° C. is preferably 4.0 or more and 10.0 or less, and more preferably 5.5 or more and 8.5 or less from the viewpoint of the stability of the antibody. At the start of the cross-linking reaction, the lower limit of the concentration of the antibody is preferably 1.0 μmol/L or more and the upper limit thereof is 1,000 μmol/L or less, and the upper limit thereof is more preferably 500 μmol/L or less.

Subsequently, the IgG-binding peptide modified with the crosslinking agent and a catalyst as necessary are added, and the mixture can be dispersed at 10° C. or higher and 30° C. or lower.

The mixing ratio of the IgG-binding peptide of the present disclosure and the antibody of the present disclosure in the mixing process is not particularly limited. The molar ratio of the IgG-binding peptide of the present disclosure to the antibody of the present disclosure can be, for example, 1:1 to 20:1, preferably 2:1 to 20:1 or 5:1 to 10:1.

In certain preferred aspects, in the mixing process, the IgG-binding peptide (molar ratio) to the antibody of the present disclosure can be mixed at 0.5 to 2.2, preferably 0.8 to 1.8. In this way, a monovalent modified antibody (that is, a complex containing one IgG-binding peptide to the antibody) can be efficiently obtained.

The mixing time (reaction time) in the mixing process is not limited as long as a cross-linking reaction occurs between the IgG-binding peptide of the present disclosure and the antibody of the present disclosure, but can be, for example, 1 minute to 5 hours, preferably 10 minutes to 2 hours.

In this way, the IgG-binding peptide can bind to the Fc region of the antibody. The antibody of the present disclosure has one binding region of the peptide per heavy chain. Therefore, the peptide can bind one or two to one antibody having two heavy chains. Then, in this separation or purification process, the unmodified antibody (that is, naked antibodies), the monovalent modified antibody (that is, an antibody modified with one IgG-binding peptide for one antibody), and the bivalent modified antibody (that is, an antibody modified with two IgG-binding peptides for one antibody) may not be separated, only the unmodified antibody may be removed from the mixture to obtain a mixture of the monovalent modified antibody and the bivalent modified antibody, or the monovalent modified antibody and the bivalent modified antibody may be isolated, concentrated, or purified, respectively. In a case where the unmodified antibody is removed from the mixture, or in a case where the monovalent modified antibody and the bivalent modified antibody are separated, it can be performed by, for example, chromatography such as gel filtration chromatography, ion exchange column chromatography, affinity chromatography, reverse phase column chromatography, or HPLC. In certain preferred aspects, at least one of the unmodified antibody, the monovalent modified antibody and the bivalent modified antibody may be separated by an IgG-BP column method (see WO2021/080008) or affinity chromatography (for example, a protein A column or a protein G column).

The IgG-BP column is a column in which an IgG-binding peptide is immobilized. The bivalent modified antibody cannot bind to the column because the binding site is already occupied by the IgG-binding peptide, and only the monovalent modified antibody exhibits affinity for the column. Therefore, the monovalent modified antibody and the bivalent modified antibody can be easily separated using the IgG-BP column.

Thus, in certain preferred aspects, there may be provided a composition containing an unmodified antibody and a monovalent modified antibody, in which the molar ratio of the unmodified antibody and the monovalent modified antibody is 4 to 47:53 to 96, preferably 4 to 30:70 to 96, more preferably 4 to 20:80 to 96, and still more preferably 4 to 10:90 to 96.

### (5) Preparation of Antibody-RI Conjugates of Present Disclosure

Preparation examples of the antibody-RI conjugate of the present disclosure will be described.

As the antibody, a humanized antibody of a HEG1 protein expressed in mesothelioma cells (for example, the humanized IgG antibodies and the human IgG antibodies) is used as described above. The antibody of the present disclosure can be maintained in buffer.

There may be a method for preparing the antibody-RI conjugates of the present disclosure, for example, by introducing radioactive iodine (¹²³I, ¹²⁵I, or ¹³¹I) as the radionuclide into a tyrosine residue of the antibody of the present disclosure. In addition, there may be a method for preparing the antibody of the present disclosure by introducing a substituent that stably binds to the radiohalogen nuclide and reacting the antibody with a radiohalogen ion.

The antibody-RI conjugate of the present disclosure can be prepared by conjugating the antibody and the chelating agent, and then chelating the metal radionuclide or the radioactive metal halide with the chelating agent.

Examples of the introducing method for the chelate site into an antibody of the present disclosure include the following methods (a) to (f):
(a) an amine coupling method (a method for modifying the amino group of the lysine residue of an antibody with a chelating agent having a carboxyl group activated with an N-hydroxysuccimidyl (NHS) group);
(b) a method for modifying a sulfhydryl (SH) group generated by partially reducing a disulfide bond between polypeptide chains at a hinge site of an antibody with a chelating agent having a maleimide group;
(c) a method for modifying a cysteine residue newly introduced into the antibody by amino acid mutation through genetic engineering with the chelating agent having the maleimide group;
(d) a method in which the azide group of an azide lysine newly introduced into the antibody by amino acid mutation through genetic engineering is modified with the chelating agent to which the alkyne (for example, Dibensylciclooctene: DBCO) is linked using a click reaction;
(e) a method for modifying glutamine introduced at a specific position of an antibody with a chelating agent having a lysine side chain using transglutaminase; and
(f) a method in which in a case where the antibody is IgG, the antibody is modified with a chelating agent by binding a chelating agent in which the IgG-binding peptide is site-specifically linked to an Fc region via the IgG-binding peptide, and then crosslinking the antibody and the IgG-binding peptide.

The reaction can be performed under conditions suitable for the reaction. Thus, the antibody and the chelating agent can be obtained.

In addition, after a radioactive metal complex in which a metal radionuclide or a radioactive metal halide is chelated with a chelating agent is obtained, a chelate may be introduced into the antibody using the radioactive metal complex in place of the chelating agent in the methods shown in (a) to (f) above.

As a preferred aspect, there may be a method for preparing an antibody-RI conjugate by modifying an antibody with a chelating agent or a chelate using the method for the above (f). Specifically, the peptide-modified antibody can be further complexed with a chelate site at which the metal radionuclide is chelated or a chelate site at which the metal radionuclide is not chelated. The complexation can be achieved by covalently linking the peptide of the peptide-modified antibody and the chelate site. The linking between the peptide of the peptide-modified antibody and the chelate site can be appropriately carried out by those skilled in the art. In certain aspects, the peptide-modified antibody and the chelate site may be linked by a click reaction.

In the process of reacting a chelating agent introduced into the antibody or the chelating agent before being introduced into the antibody with the metal radionuclide to form a chelate (complex formation process), the metal radionuclide is preferably used in the form of an ionizable radioactive metal compound, and more preferably used in the form of a radioactive metal ion, from the viewpoint of enhancing the chelate formation efficiency (hereinafter, these forms are also collectively referred to as a “radioactive metal source”).

In the complex formation process, the order of addition of the chelating agent and the radioactive metal source is not limited as long as complex formation with radioactive metal ions is possible. For example, one of the chelating agent and the radioactive metal source may be added to a reaction vessel containing a solvent, and then the other may be added and reacted, or the other may be added to a dispersion in which one of the chelating agent and the radioactive metal source is dispersed in a solvent and reacted. Alternatively, they may be simultaneously added to a reaction vessel containing a solvent to cause a reaction.

As reaction conditions in the complex formation process, for example, the following conditions can be set. As the solvent, for example, water, physiological saline, a buffer such as a sodium acetate buffer, an ammonium acetate buffer, a phosphate buffer, a phosphate buffer saline, a Tris buffer, a HEPES buffer, or a tetramethylammonium acetate buffer, a water-soluble organic solvent such as an alcohol having 1 to 5 carbon atoms, acetonitrile, N, N-dimethylformamide, tetrahydrofuran, dimethyl sulfoxide, or acetone, or a mixed solvent thereof can be used. The reaction temperature may be, for example, room temperature (25° C.) or may be under heating conditions, but the upper limit is preferably 120° C. or lower, more preferably 90° C. or lower, still more preferably 50° C. or lower, and still more preferably 40° C. or lower from the viewpoint of achieving both suppression of decomposition of the chelating agent and improvement of complex formation efficiency. The lower limit is not particularly limited as long as it is a temperature at which complex formation is possible, but is preferably 0° C. or higher, more preferably 10° C. or higher, still more preferably 15° C. or higher, still more preferably 20° C. or higher, still more preferably 30° C. or higher, and particularly preferably 35° C. or higher. It is preferably heated to 30° C. or higher and 100° C. or lower, and more preferably 37° C. or higher and 90° C. or less. The lower limit of the reaction time is preferably 5 minutes or longer, more preferably 10 minutes or longer, still more preferably 20 minutes or longer, still more preferably 30 minutes or longer, particularly preferably 60 minutes or longer, and the upper limit is preferably 300 minutes or shorter, more preferably 150 minutes or shorter, still more preferably 120 minutes or shorter, particularly preferably 60 minutes or shorter, preferably 10 minutes or longer and 150 minutes or shorter, further preferably 30 minutes or longer and 60 minutes or shorter on condition that the reaction temperature is as described above.

As the radioactive metal source in the complex formation process, for example, a solution in which radioactive metal ions are dispersed or dissolved in a solvent mainly composed of water can be used.

The amount of the reaction solution in the complex formation process is not particularly limited, but from the viewpoint of practicality, the lower limit at the start of this process is preferably 0.01 mL or more, more preferably 0.1 mL or more, still more preferably 1 mL or more, and the upper limit is preferably 1,000 mL or less, more preferably 100 mL or less, still more preferably 10 mL or less, for example, preferably 0.01 mL or more and 100 mL or less. In addition, the lower limit of the concentration of the ligand and the radioactive metal ion in the reaction solution is preferably 0.01 μmol/L or more, more preferably 0.1 μmol/L or more, still more preferably 1 μmol/L or more, and the upper limit is preferably 10,000 μmol/L or less, more preferably 1,000 μmol/L or less, still more preferably 100 μmol/L or less, for example, preferably 1 μmol/L or more and 100 μmol/L or less, at the start of this process, from the viewpoint of the yield of a target chelate. The molar ratio of the chelating agent and the radioactive metal ion varies depending on the type of the chelating agent and the radioactive metal ion to be used, but the lower limit of the molar ratio of the chelating agent and the radioactive metal ion is preferably 10/1 or more, more preferably 100/1 or more, still more preferably 200/1 or more, 300/1 or more, still more preferably 500/1 or more, and the upper limit is preferably 10000/1 or less, more preferably 9,000/1 or less, still more preferably 8,000/1 or less, still more preferably 7,000/1 or less, and preferably 200/1 or more and 10,000/1 or less, particularly preferably 500/1 or more and 7,000/1 or less.

In certain preferred aspects, from the viewpoint that the metal radionuclide to be labeled can be used without particular limitation according to the use and purpose of the antibody-RI conjugate, the process of reacting the chelating agent with the metal radionuclide to form a chelating agent (complex formation process) is provided before the process of complexing the antibody and the metal radionuclide (antibody labeling process). In this case, from the viewpoint of enhancing the chelate formation efficiency without depending on the combination of the chelating agent and the metal radionuclide, it is preferable that the chelating agent and the metal radionuclide are heated and reacted in the complex formation process. In this regard, complex formation may not proceed well under non-heating conditions depending on the combination of the chelating agent and the metal radionuclide, but complex formation can proceed efficiently without depending on the combination of the chelating agent and the radioactive metal by forming the complex coordinated with the radioactive metal ion under heating conditions. As a more preferred aspect, the peptide of the peptide-modified antibody can be linked to the chelate site at which the metal radionuclide is chelated. This can be beneficial, such as in a case where the antibody is denatured under chelating reaction conditions of the metal radionuclide to the chelate site. As this aspect, the method described in WO2021/075546 can be used.

As one aspect, from the viewpoint of achieving both improvement in the reaction efficiency of the reaction between the chelating agent and the radioactive metal ion and improvement in the reaction efficiency of the click reaction between the obtained complex and the peptide-modified antibody, the chelating agent preferably has a chelating moiety which is a site to which the radioactive metal ion is coordinated and a modification site bonded to the first atomic group, as represented by Formula (3a) below. Thereby, it is possible to prepare an antibody-RI conjugate including the ligand represented by Formula (ii) described above as a chelate site.

As represented by Formula (3a) below, the modification site (in Formula (3a), Rm is used) is bonded to the above-described chelating moiety (in Formula (3a), Ch is used), and the above-described first atomic group (in Formula (3a), AG is used) is bonded. In Formula (3a), Rm is a linear or branched chain, is substituted or unsubstituted, and is an atomic group having a total carbon atom number of 10 or more and 50 or less.

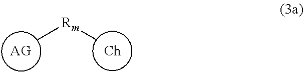

The mode of binding of the modification site Rm to the chelating moiety Ch is not particularly limited as long as the ligand and the radioactive metal ion can form a complex, but from the viewpoint of efficiently forming a complex between the ligand and the radioactive metal ion, it is preferable that the modification site Rm and the chelating moiety Ch form a thiourea bond and bind to each other, or the modification site Rm and the chelating moiety Ch form an amide bond and bind to each other. In addition, from the viewpoint of enhancing the labeling efficiency between the radioactive metal complex and the antibody, the modification site Rm is preferably bonded to the first atomic group.

From the viewpoint of further enhancing the labeling efficiency between the radioactive metal complex and the antibody, it is preferable that the modification site has a structure represented by Formula (P2) below bonded to the structure represented by Rm in Formula (3a). The structure is a structure derived from ethylene glycol, and in Formula (P2), r is preferably an integer of 2 or more and 50 or less, and more preferably an integer of 2 or more and 30 or less.

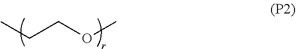

By performing the above-described complex formation process using this ligand, the radioactive metal complex in which the metal radionuclide is chelated with a ligand can be obtained. The antibody-RI conjugate of the present disclosure can be prepared by performing a click reaction between the resulting radioactive metal complex and the antibody modified with the IgG peptide having the second atomic group introduced thereinto (linker modified antibody) (antibody labeling process). This click reaction can be achieved by a combination of an atomic group containing dibenzylcyclooctyne (DBCO) as the alkyne of the first atomic group and an atomic group containing the azide group as the azide of the second atomic group, or a combination of an atomic group containing 1,2,4,5-tetrazine as the first atomic group and an atomic group containing trans-cyclooctene (TCO) as the alkene of the second atomic group. Preferably, it can be achieved by a combination of DBCO and the azide group. By introducing the azide group into the peptide and introducing DBCO into the chelating moiety of the ligand, the click reaction can be utilized.

The present antibody labeling process is not particularly limited as long as the click reaction can be performed. For example, one of the radioactive metal complex and the linker modified antibody may be added to a reaction vessel containing a solvent, and then the other may be added to cause a reaction, or the other may be added to a dispersion in which one of the radioactive metal complex and the linker modified antibody is dispersed in a solvent to cause a reaction. Alternatively, they may be simultaneously added to a reaction vessel containing a solvent to cause a reaction.

As the solvent, a solvent containing water can be used, and for example, water, physiological saline, or a buffer such as a sodium acetate buffer, an ammonium acetate buffer, a phosphate buffer saline, a phosphate buffer, a trishydroxymethylaminomethane buffer (hereinafter, simply referred to as “Tris buffer”), a 4-(2-hydroxyethyl)-1 piperazineethanesulfonic acid buffer (hereinafter, simply referred to as “HEPES buffer”), or a tetramethylammonium acetate buffer can be used. In a case where a buffer is used, the lower limit of the pH at 25° C. is preferably 3.5 or more, more preferably 4.0 or more, still more preferably 4.5 or more, still more preferably 5.0 or more, and particularly preferably 5.5 or more, and the upper limit thereof is preferably 10.0 or less, more preferably 9.5 or less, still more preferably 9.0 or less, more still more preferably 8.5 or less, and particularly preferably 8.0 or less, and is set to 4.0 or more and 10.0 or less as a preferable range and 5.5 or more and 8.5 or less as a more preferable range, from the viewpoint of achieving both the stability of the complex and the antibody and the binding efficiency thereof.

From the viewpoint of enhancing the binding efficiency between the radioactive metal complex and the linker modified antibody while preventing unintended denaturation of the antibody, the upper limit of the reaction temperature in the click reaction in this process is preferably 120° C. or lower, more preferably 90° C. or lower, still more preferably 50° C. or lower, and still more preferably 40° C. or lower. The lower limit of the reaction temperature is not particularly limited as long as it is a temperature capable of click reaction, but is preferably 10° C. or higher, more preferably 15° C. or higher, still more preferably 20° C. or higher, still more preferably 30° C. or higher, and particularly preferably 35° C. or higher. The lower limit of the reaction time of the click reaction is preferably 5 minutes or longer, more preferably 10 minutes or longer, still more preferably 20 minutes or longer, still more preferably 30 minutes or longer, and particularly preferably 60 minutes or longer, and the upper limit is preferably 36 hours or shorter, more preferably 24 hours or shorter, still more preferably 20 hours or shorter, and particularly preferably 15 hours or shorter, and the preferable range is 5 minutes or longer and 24 hours or shorter, and the further preferable range is 10 minutes or longer and 20 hours or shorter on condition that the reaction temperature is the above-described reaction temperature.

The reaction solution volume is not particularly limited, but from the viewpoint of practicality, the lower limit at the start of this process is preferably 0.01 mL or more, more preferably 0.1 mL or more, still more preferably 1 mL or more, and the upper limit is preferably 1,000 mL or less, more preferably 100 mL or less, still more preferably 10 mL or less, for example, preferably 0.1 mL or more and 10 mL or less. In addition, the lower limit of the concentration of the radioactive metal complex and the linker modified antibody in the reaction solution is preferably 0.01 μmol/L or more, more preferably 0.1 μmol/L or more, still more preferably 1 μmol/L or more, and the upper limit thereof is preferably 10,000 μmol/L or less, more preferably 1000 μmol/L or less, still more preferably 100 μmol/L or less, for example, preferably 1 μmol/L or more and 100 μmol/L or less, at the start of this process, from the viewpoint of the yield of the target antibody-RI conjugate.

The obtained antibody-RI conjugate may be used as it is, or may be purified using a filtration filter, a membrane filter, a column packed with various fillers, chromatography, or the like.

In the antibody-RI conjugate obtained in this aspect, a specific site of the antibody is specifically modified with the IgG-binding peptide. The chelate site is directly or indirectly linked to the IgG-binding peptide, and has a binding site, preferably formed by a click reaction, between the IgG-binding peptide and the chelate site. The binding site is preferably a chemical structure derived from a first atomic group linked to the chelate site and a second atomic group linked to the IgG-binding peptide. As such a chemical structure, for example, the binding site has a structure including a substituted triazole skeleton or a structure including a substituted pyridazine skeleton formed. Examples of the structure including a substituted skeleton include a structure in which a structure including at least one of a substituent, an aliphatic ring, and an aromatic ring is bonded to a triazole skeleton or a pyridazine skeleton, and which has a binding site with the modification site or the chelating moiety and a binding site with a peptide.

As a specific structure of the binding site, for example, in a case where the first atomic group and the second atomic group are a combination of an atomic group containing DBCO and an atomic group containing an azide group, a structure including a triazole skeleton represented by Formula (10a) or Formula (10b) below is formed depending on a reaction reagent to be used, and these may be contained in an arbitrary ratio since they are in an isomeric relationship. In addition, in a case where the first atomic group and the second atomic group are a combination of an atomic group containing 1,2,4,5-tetrazine and an atomic group containing TCO, a structure containing a pyridazine skeleton shown in Formula (10c) below is formed depending on a reaction reagent to be used. In Formulae (10a) and (10b), Ria represents a binding site with a modification site or a chelating moiety, and R_{2A} represents a binding site with a peptide. In Formula (10c), one of R_{3A} and R_{4A} represents a hydrogen atom, a methyl group, a phenyl group or a pyridyl group, the other represents a binding site with a modification site or a chelating moiety, and R_{5A} represents a binding site with a peptide.

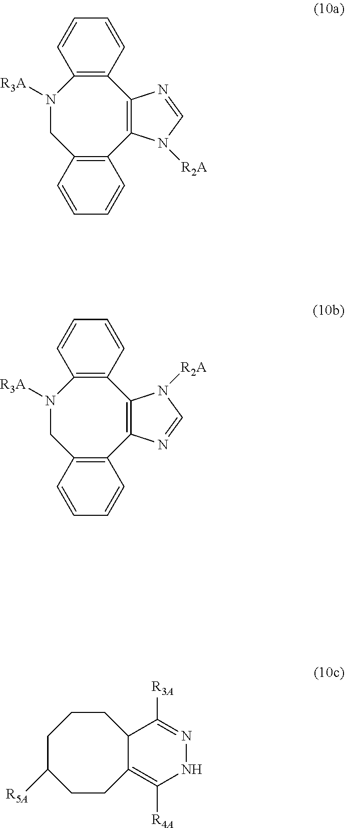

The triazole skeleton-containing structures represented by Formulae (A) to (J) and (10a) and (10b) above and the pyridazine skeleton-containing structure represented by Formula (10c) may be substituted with a substituent such as a halogen atom, a saturated or unsaturated alkyl group, a hydroxy group, an aldehyde group, a carboxy group, an acyl group, an amino group, a nitro group, an ester group, an isothiocyanate group, a thioxy group, a cyano group, an amide group, an imide group, a phosphate group, a phenyl group, a benzyl group, or a pyridyl group. One of these substituents may be substituted alone, or two or more of these substituents may be combined and substituted.

The thus obtained antibody-RI conjugate of the present disclosure can also be used for preparation of a radiopharmaceutical composition containing the antibody-RI conjugate as an active ingredient. The radiopharmaceutical composition refers to a composition containing an antibody-RI conjugate of the present disclosure or a derivative thereof, in which the composition is in a form suitable for injection into a living body. The radiopharmaceutical composition can be produced, for example, by dissolving the antibody-RI conjugate produced by the above-described method in a solvent mainly composed of water and substantially isotonic with a living body. In this case, the radiopharmaceutical composition is preferably in the form of an aqueous solution, and may contain other pharmaceutically acceptable components as necessary. The radiopharmaceutical composition is injected to a living body orally or parenterally such as intravenously, subcutaneously, intraperitoneally, or intramuscularly, and is used for treatment of a disease, diagnosis of a disease, detection of a lesion, or the like.

The antibody-RI conjugate of the present disclosure, in certain aspects is a complex including an antibody and a metal radionuclide, in which the antibody is a humanized antibody capable of binding to a human HEG1 protein expressed in mesothelioma cells, the humanized antibody is an IgG, the metal radionuclide is chelated to a chelate site, the chelate site includes DOTAs (preferably DOTA or DOTA-GA), the chelate site is covalently linked to the humanized IgG via a linker including an IgG-binding peptide, and the metal radionuclide is ²²⁵Ac.

In certain aspects, a complex comprising an antibody and a metal radionuclide, in which the antibody is a humanized antibody capable of binding to a human HEG1 protein expressed in mesothelioma cells, the humanized antibody is an IgG, the metal radionuclide is chelated to a chelate site, the chelate site includes DOTAs (preferably DOTA or DOTA-GA), the chelate site is covalently linked to the humanized IgG via a linker including an IgG-binding peptide, and the metal radionuclide is ⁸⁹Zr.

In certain preferred aspects, the complex including the antibody and the metal radionuclide may include the structure of Formula (X) below.

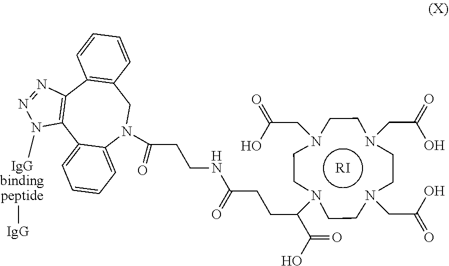

{wherein,
RI (circle labeled RI) represents the metal radionuclide chelated to a chelate site, the IgG-binding peptide is cross-linked with a crosslinkage agent, preferably PEGylated, and IgG represents a humanized IgG antibody capable of binding to a human HEG1 protein expressed in mesothelioma cells.}

In certain preferred aspects, the complex including the antibody and the metal radionuclide may include the structure of Formula (XI) below.

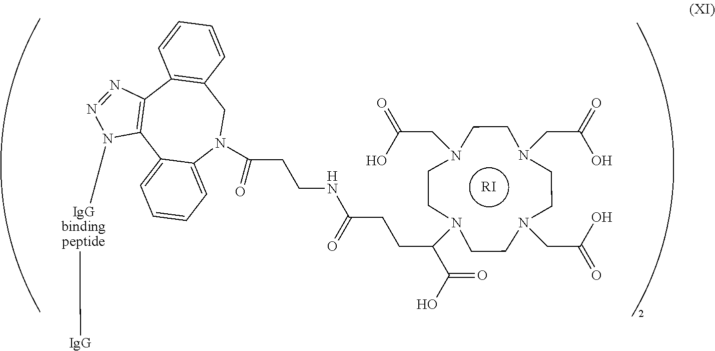

{wherein, RI (circle labeled RI) represents the metal radionuclide chelated to a chelate site, the IgG-binding peptide is cross-linked with a crosslinkage agent, preferably PEGylated, and IgG represents a humanized IgG antibody capable of binding to a human HEG1 protein expressed in mesothelioma cells.}

In certain aspects, the metal radionuclide can be a therapeutic metal radionuclide or a diagnostic metal radionuclide. In certain preferred aspects, the metal radionuclide may be an α-ray emitting nuclide, more preferably ²²⁷Th or ²²⁵Ac, and still more preferably ²²⁵Ac. In certain preferred aspects, the metal radionuclide may be ¹¹¹In (indium), ⁸⁹Zr (zirconium), ^{67/68}Ga (gallium), ^{99m}Tc (technetium), ⁶⁴Cu (copper), preferably ¹¹¹In, and ⁸⁹Zr. In certain preferred aspects, it may be ¹¹¹In, ⁸⁹Zr, ⁶⁴Cu, ^{67/68}Ga, and ^{99m}Tc, for example, 89% r.

In this aspect, the IgG-binding peptide is present in proximity to the lysine of the IgG antibody, which correspond to lysines 246th and 248th (sixteenth and eighteenth of the amino acid sequence set forth in SEQ ID NO: 215, and tenth and twelfth of the amino acid sequence set forth in SEQ ID NO: 220, respectively) of the IgG antibody and can be cross-linked between Xaa1 of the IgG-binding peptide and any of the above lysine residues (preferably a lysine residue corresponding to the 248-th lysine residue). In this aspect, for example, the IgG antibody may be an IgG1 antibody. In addition, in this aspect, for example, the IgG-binding peptide can be a peptide having the amino acid sequence set forth in SEQ ID NO: 186 {wherein, Xaa can be K}. In certain preferred aspects, the IgG-binding peptide has an N-terminal or C-terminal amino acid linked to the triazole ring in Formula (X).

In certain preferred aspects, the humanized IgG antibody can be an antibody that binds to a peptide having the amino acid sequence set forth in SEQ ID NO: 182 (SKSPSLVSLPT). The partial peptide can be, for example, a peptide produced by mesothelioma cells (for example, ACC-MESO4 cell line). The peptide can be obtained as a fusion protein by being linked to the N-terminal side of a protein in which a GPI anchor signal is linked to the N-terminal of human SLURP1, for example, and can be used for evaluation of binding to an antibody. In certain preferred aspects, the human antibody can be any of the human antibodies described above.

In certain preferred aspects, the humanized IgG antibody can be any of the humanized antibodies described above. In certain preferred aspects, the antibody of the present disclosure may include a heavy chain variable region set forth in SEQ ID NO: 6 and a light chain variable region set forth in SEQ ID NO: 21.

Non-limiting examples of the heavy chain constant region of the IgG antibody include the heavy chain constant regions of IgG1, IgG2, IgG3, IgG4, and IgG4PE, and the heavy chain constant regions of IgG1 and IgG4PE are more preferred, and the heavy chain constant region of IgG4PE can be further preferably used. The hinge region of the IgG1 may include, for example, the amino acid sequence of SEQ ID NO: 216. The hinge region of the IgG4 may, for example, have the amino acid sequence of SEQ ID NO: 217, and the hinge region of the IgG4PE may, for example, have the amino acid sequence of SEQ ID NO: 218. The CH2 region of the IgG1 may include, for example, the amino acid sequence of SEQ ID NO: 219. The CH2 regions of IgG4 and IgG4PE may, for example, have the amino acid sequence of SEQ ID NO: 220.

Non-limiting examples of light chain constant regions of IgG antibodies include light chain constant regions of kappa or lambda chains, for example, light chain constant regions of kappa chains.

The humanized antibody of the present disclosure may or may not have internalizing activity for therapeutic and diagnostic purposes. As long as the antibody-RI conjugate of the present disclosure binds to the cell membrane surface, since cells are damaged by radiation such as α-rays or β-rays generated from the metal radionuclide contained, the antibody may not have an internalizing activity.

### <Test Method for Mesothelioma>

According to the present disclosure, the complex of the antibody and the metal radionuclide (antibody-RI conjugate) of the present disclosure can be used to detect mesothelioma (or mesothelioma cell). Thus, according to the present disclosure, there is provided a method for detecting mesothelioma (or mesothelioma cells) in a subject, including injecting to the subject an effective amount of the complex. According to the present disclosure, there is also provided a composition (for example, a diagnostic agent) containing the antibody-RI conjugate for use in a method for detecting mesothelioma (or mesothelioma cells) in a subject.

The antibody-RI conjugates or compositions of the present disclosure can be injected parenterally (for example, intravenous injection, intraperitoneal injection, or intrapleural injection) to a subject for the purpose of diagnosing mesothelioma or detecting mesothelioma cells. The composition of the present disclosure can be in a dosage form suitable for the route of injection. The injection method and the dosage form can be appropriately selected by those skilled in the art depending on the sex, age, weight, symptom, and the like of the patient.

The composition for the diagnosis of mesothelioma or the detection of mesothelioma cells can be formulated according to routine methods (see, for example, Remington's Pharmaceutical Science, latest edition, Mark Publishing Company, Easton, USA) and may include both pharmaceutically acceptable carriers and additives.

Examples of carriers and pharmaceutical additives that may be included in the composition for the diagnosis of mesothelioma or the detection of mesothelioma cells include water, pharmaceutically acceptable organic solvents, collagen, polyvinyl alcohol, polyvinylpyrrolidone, carboxyvinyl polymers, sodium carboxymethyl cellulose, sodium polyacrylate, sodium alginate, water-soluble dextran, sodium carboxymethyl starch, pectin, methyl cellulose, ethyl cellulose, xanthan gum, gum arabic, casein, agar, polyethylene glycol, diglycerin, glycerin, propylene glycol, petrolatum, paraffin, stearyl alcohol, stearic acid, human serum albumin (HSA), mannitol, sorbitol, lactose, and surfactants acceptable as pharmaceutical additives.

The actual additive is selected alone or in appropriate combination from the above depending on the dosage form of the composition for diagnosis of mesothelioma or detection of mesothelioma cells, but is not limited thereto. For example, in a case where the additive is used as a preparation for injection, the antibody-RI conjugate complex of the present disclosure can be dissolved in a solution, for example, physiological saline, a buffer, a glucose solution, or the like, and a container adsorption inhibitor, for example, polysorbate 80, polysorbate 20, gelatin, human serum albumin, or the like can be added thereto. Alternatively, it may be freeze-dried to form a dosage form that is dissolved and reconstituted before use, and as a stabilizer for freeze-drying, for example, a sugar alcohol such as mannitol or glucose and/or a saccharide can be used.

The effective injected dose and injection interval of the composition for diagnosis of mesothelioma or detection of mesothelioma cells can be appropriately selected according to the sex, age, weight, symptom, and the like of the patient.

According to the present disclosure, there is provided a method for detecting mesothelioma or mesothelioma cells in a subject suffering from or having potential for mesothelioma, the method including
reacting the subject with a complex of the present disclosure and measuring the level or presence of radioactivity derived from the metal radionuclide of the subject. The presence of radioactivity may indicate the presence of the mesothelioma or the mesothelioma cells. Thus, the mesothelioma or the mesothelioma cells can be detected based on the presence of radioactivity. In a case where the mesothelioma or the mesothelioma cells are detected, the physician can diagnose that the subject is suffering from the mesothelioma. Herein, the method for detecting mesothelioma can be read as “a method of diagnosing mesothelioma”, “a method for diagnosing mesothelioma”, “a nuclear medicine imaging method for detecting mesothelioma”, “a method for obtaining preliminary information for diagnosing mesothelioma”, or “a method for detecting mesothelioma cells”. The method may also be an in vitro method. The method may also be an industrially applicable method. Also, the method may not include a process of diagnosis by a physician.

The methods described above may include reacting a complex of the present disclosure to a biological sample obtained from a subject, washing away the complex that did not bind to the biological sample, and measuring the level or presence of radioactivity derived from the metal radionuclide in the biological sample. The biological samples that may be used in the present method include tissues including mesothelioma cells and body fluids such as pleural effusion, ascites, and blood (for example, serum or plasma).

The method for measuring the level or presence of radioactivity is not particularly limited, and any method known to those skilled in the art can be used. For example, image analysis such as SPECT/CT may be performed, or the level or presence of radioactivity may be measured using a detector such as a scintillation counter.

The process of determining or detecting the presence or absence of cancer development in a subject based on the level or presence of radioactivity is not particularly limited, and any method known to those skilled in the art can be used. For example, if the level of radioactivity in a sample from a subject subjected to the methods of the present invention is significantly higher for multiple, for example, 2 or more, 3 or more, 4 or more, preferably 5 or more samples from a subject known not to suffer from cancer, it can be determined that the subject is suffering from or is likely to suffer from cancer.

According to the present disclosure, there is provided a method for determining whether a subject is suffering from mesothelioma, including injecting to the subject an effective amount of an antibody-RI conjugate of the present disclosure, followed by measuring the level or presence of radioactivity derived from a radionuclide in the subject. In this method, the presence of radioactivity derived from the radionuclide indicates that the subject is suffering from or is likely to suffer from mesothelioma. In addition, the level of radioactivity derived from the radionuclide in the tissue containing the mesothelial cells (tissues such as, for example, pleura and peritoneum) having a significant difference from the level in the normal tissue indicates that the subject is suffering from or is likely to suffer from mesothelioma. Conversely, the level of radioactivity derived from the radionuclide in tissue containing mesothelial cells (tissues such as, for example, pleura and peritoneum) does not have a significant difference from the level in normal tissue, indicating that the subject is not suffering from mesothelioma or is not likely to suffer from mesothelioma. Thus, according to the method for the present disclosure, it can be determined whether the subject is suffering from mesothelioma based on the level or presence of radioactivity. The present method is preferably a nuclear medicine imaging method. By the present method, the distribution status of an antigen or an antibody in a subject or the distribution status and/or biokinetics of an antibody-RI conjugate can be inferred. In addition, in the present method, the distribution status and/or progress status of mesothelioma in the subject can be inferred by measuring the level and distribution of radioactivity derived from the radionuclide.

According to the present disclosure, the antibody-RI conjugate of the present disclosure for use in the above methods is provided. According to the present disclosure, the composition (or a diagnostic agent and a nuclear medicine image diagnostic agent) containing the antibody-RI conjugates of the present disclosure for use in the methods described above is provided. According to the present disclosure, the use of an antibody-RI conjugate of the present disclosure in the production of a composition containing the antibody-RI conjugate of the present disclosure (or a diagnostic agent and a nuclear medicine image diagnostic agent) for use in the above method is provided. The complex used for diagnosis includes a diagnostic metal radionuclide as the radionuclide.

The methods of the present disclosure may further include treating mesothelioma in a subject.

### <Method for Treating Mesothelioma in Subject>

According to the present disclosure, a method for treating mesothelioma in a subject is provided. According to the present disclosure, a method includes injecting to a subject an effective amount of an antibody-RI conjugate of the present disclosure. The subject may be a subject with mesothelioma. The subject may be a subject diagnosed as having mesothelioma. The subject may be a subject determined to have mesothelioma by the methods of the present disclosure.

The injection may be performed by parenteral injection (for example, intraperitoneal injection, intrapleural injection, intratumoral injection, intravenous injection, or the like). The injected dose can be appropriately determined by a physician in consideration of the condition, weight, sex, and age of the subject. The injection may be a single injection or multiple injections.

According to the present disclosure, the antibody-RI conjugate of the present disclosure is provided for use in the above method.

### <Method for Determining Therapeutic Effect>

The therapeutic effect can be determined by the method for detecting the mesothelioma of the present disclosure or the like. Methods of determining a therapeutic effect of a cancer therapy can be provided, the method including injecting to a subject an effective amount {wherein, the radionuclide is a diagnostic metal radionuclide} of an antibody-RI conjugate of the present disclosure before and after the cancer therapy, and measuring the level and/or biodistribution of radioactivity derived from the radionuclide, in which the complex includes a diagnostic metal radionuclide, and comparing the level and/or biodistribution before and after injection. In a case where the level is reduced later than before the cancer therapy, it can be determined that there is a therapeutic effect. Alternatively, in a case where the biodistribution is reduced later than before the treatment process, it can be determined that there is a therapeutic effect. In a case where the therapeutic effect is determined, the subject may be further injected an effective amount of an antibody-RI conjugate of the present disclosure {here, the complex includes a therapeutic metal radionuclide}. In addition, in a case where the presence of radioactivity derived from the diagnostic metal radionuclide cannot be detected after the treatment process, the treatment may be stopped. The cancer therapy includes, but is not particularly limited to, for example, surgical resection, chemotherapy, and radiation therapy, but in certain aspects, cancer therapy may include injecting to a subject an antibody-RI conjugate of the present disclosure, where the radionuclide is a therapeutic metal radionuclide.

According to the present disclosure, the antibody-RI conjugate of the present disclosure is provided for use in the above method.

### <Kit>

According to the present disclosure, a kit including the linker modified antibody of the present disclosure is provided. One aspect of the linker modified antibody is a modified antibody in which the IgG antibody is modified with a linker, which is a humanized antibody capable of binding to a human HEG1 protein expressed in mesothelioma cells among the antibodies of the present disclosure as an IgG antibody, in which the linker includes an IgG-binding peptide described above, and the peptide is crosslinked with the IgG antibody. In this modified antibody, the linker may contain the chelating agent described above or may contain the second atomic group described above. In a case where the linker contains a second atomic group, the kit may further have a ligand containing the first atomic group described above. The kit of the present disclosure is used for the production of the antibody-RI conjugate of the present disclosure.

The above embodiments of the present invention include the following technical ideas.

1: A complex comprising
an antibody or antigen-binding fragment thereof; and
a radionuclide, in which
the antibody is a humanized antibody capable of binding a human HEG1 protein expressed on mesothelioma cells.

2: The complex according to above 1, in which
the antibody includes a heavy chain variable region including a heavy chain CDR1 having an amino acid sequence set forth in SEQ ID NO: 37, a heavy chain CDR2 having an amino acid sequence set forth in SEQ ID NO: 43, and a heavy chain CDR3 having an amino acid sequence set forth in SEQ ID NO: 51, and a light chain variable region including a light chain CDR1 having an amino acid sequence set forth in SEQ ID NO: 62, a light chain CDR2 having an amino acid sequence set forth in SEQ ID NO: 75, and a light chain CDR3 having an amino acid sequence set forth in SEQ ID NO: 82.

3: The complex according to above 1 or 2, in which
the antibody includes a heavy chain variable region including a heavy chain CDR1 having an amino acid sequence set forth in SEQ ID NO: 37, a heavy chain CDR2 having an amino acid sequence set forth in SEQ ID NO: 43, and a heavy chain CDR3 having an amino acid sequence set forth in SEQ ID NO: 51, and a light chain variable region including a light chain CDR1 having an amino acid sequence set forth in SEQ ID NO: 63, a light chain CDR2 having an amino acid sequence set forth in SEQ ID NO: 75, and a light chain CDR3 having an amino acid sequence set forth in SEQ ID NO: 82.

4: The complex according to any one of above 1 to 3, in which
the heavy chain variable region includes
(1) a heavy chain CDR1 having an amino acid sequence set forth in SEQ ID NO: 89, a heavy chain CDR2 having an amino acid sequence set forth in SEQ ID NO: 90, and a heavy chain CDR3 having an amino acid sequence set forth in SEQ ID NO: 91,
(2) a heavy chain CDR1 having an amino acid sequence set forth in SEQ ID NO: 92, a heavy chain CDR2 having an amino acid sequence set forth in SEQ ID NO: 93, and a heavy chain CDR3 having an amino acid sequence set forth in SEQ ID NO: 94,
(3) a heavy chain CDR1 having an amino acid sequence set forth in SEQ ID NO: 95, a heavy chain CDR2 having an amino acid sequence set forth in SEQ ID NO: 96, and a heavy chain CDR3 having an amino acid sequence set forth in SEQ ID NO: 97,
(4) a heavy chain CDR1 having an amino acid sequence set forth in SEQ ID NO: 98, a heavy chain CDR2 having an amino acid sequence set forth in SEQ ID NO: 99, and a heavy chain CDR3 having an amino acid sequence set forth in SEQ ID NO: 100,
(5) a heavy chain CDR1 having an amino acid sequence set forth in SEQ ID NO: 101, a heavy chain CDR2 having an amino acid sequence set forth in SEQ ID NO: 102, and a heavy chain CDR3 having an amino acid sequence set forth in SEQ ID NO: 103,
(6) a heavy chain CDR1 having an amino acid sequence set forth in SEQ ID NO: 104, a heavy chain CDR2 having an amino acid sequence set forth in SEQ ID NO: 105, and a heavy chain CDR3 having an amino acid sequence set forth in SEQ ID NO: 106,
(7) a heavy chain CDR1 having an amino acid sequence set forth in SEQ ID NO: 110, a heavy chain CDR2 having an amino acid sequence set forth in SEQ ID NO: 111, and a heavy chain CDR3 having an amino acid sequence set forth in SEQ ID NO: 112, or
(8) a heavy chain CDR1 having an amino acid sequence set forth in SEQ ID NO: 113, a heavy chain CDR2 having an amino acid sequence set forth in SEQ ID NO: 114, and a heavy chain CDR3 having an amino acid sequence set forth in SEQ ID NO: 115.

5: The complex according to any one of above 1 to 4, in which
the light chain variable region includes
(1) a light chain CDR1 having an amino acid sequence set forth in SEQ ID NO: 116, a light chain CDR2 having an amino acid sequence set forth in SEQ ID NO: 117, and a light chain CDR3 having an amino acid sequence set forth in SEQ ID NO: 118,
(2) a light chain CDR1 having an amino acid sequence set forth in SEQ ID NO: 119, a light chain CDR2 having an amino acid sequence set forth in SEQ ID NO: 120, and a light chain CDR3 having an amino acid sequence set forth in SEQ ID NO: 121,
(3) a light chain CDR1 having an amino acid sequence set forth in SEQ ID NO: 122, a light chain CDR2 having an amino acid sequence set forth in SEQ ID NO: 123, and a light chain CDR3 having an amino acid sequence set forth in SEQ ID NO: 124,
(4) a light chain CDR1 having an amino acid sequence set forth in SEQ ID NO: 128, a light chain CDR2 having an amino acid sequence set forth in SEQ ID NO: 129, and a light chain CDR3 having an amino acid sequence set forth in SEQ ID NO: 130,
(5) a light chain CDR1 having an amino acid sequence set forth in SEQ ID NO: 131, a light chain CDR2 having an amino acid sequence set forth in SEQ ID NO: 132, and a light chain CDR3 having an amino acid sequence set forth in SEQ ID NO: 133,
(6) a light chain CDR1 having an amino acid sequence set forth in SEQ ID NO: 134, a light chain CDR2 having an amino acid sequence set forth in SEQ ID NO: 135, and a light chain CDR3 having an amino acid sequence set forth in SEQ ID NO: 136,
(7) a light chain CDR1 having an amino acid sequence set forth in SEQ ID NO: 137, a light chain CDR2 having an amino acid sequence set forth in SEQ ID NO: 138, and a light chain CDR3 having an amino acid sequence set forth in SEQ ID NO: 139,
(8) a light chain CDR1 having an amino acid sequence set forth in SEQ ID NO: 140, a light chain CDR2 having an amino acid sequence set forth in SEQ ID NO: 141, and a light chain CDR3 having an amino acid sequence set forth in SEQ ID NO: 142,
(9) a light chain CDR1 having an amino acid sequence set forth in SEQ ID NO: 143, a light chain CDR2 having an amino acid sequence set forth in SEQ ID NO: 144, and a light chain CDR3 having an amino acid sequence set forth in SEQ ID NO: 145,
(10) a light chain CDR1 having an amino acid sequence set forth in SEQ ID NO: 146, a light chain CDR2 having an amino acid sequence set forth in SEQ ID NO: 147, and a light chain CDR3 having an amino acid sequence set forth in SEQ ID NO: 148,
(11) a light chain CDR1 having an amino acid sequence set forth in SEQ ID NO: 149, a light chain CDR2 having an amino acid sequence set forth in SEQ ID NO: 150, and a light chain CDR3 having an amino acid sequence set forth in SEQ ID NO: 151,
(12) a light chain CDR1 having an amino acid sequence set forth in SEQ ID NO: 152, a light chain CDR2 having an amino acid sequence set forth in SEQ ID NO: 153, and a light chain CDR3 having an amino acid sequence set forth in SEQ ID NO: 154,
(13) a light chain CDR1 having an amino acid sequence set forth in SEQ ID NO: 155, a light chain CDR2 having an amino acid sequence set forth in SEQ ID NO: 156, and a light chain CDR3 having an amino acid sequence set forth in SEQ ID NO: 157,
(14) a light chain CDR1 having an amino acid sequence set forth in SEQ ID NO: 158, a light chain CDR2 having an amino acid sequence set forth in SEQ ID NO: 159, and a light chain CDR3 having an amino acid sequence set forth in SEQ ID NO: 160,
(15) a light chain CDR1 having an amino acid sequence set forth in SEQ ID NO: 161, a light chain CDR2 having an amino acid sequence set forth in SEQ ID NO: 162, and a light chain CDR3 having an amino acid sequence set forth in SEQ ID NO: 163,
(16) a light chain CDR1 having an amino acid sequence set forth in SEQ ID NO: 173, a light chain CDR2 having an amino acid sequence set forth in SEQ ID NO: 174, and a light chain CDR3 having an amino acid sequence set forth in SEQ ID NO: 175,
(17) a light chain CDR1 having an amino acid sequence set forth in SEQ ID NO: 176, a light chain CDR2 having the amino acid sequence set forth in SEQ ID NO: 177, and a light chain CDR3 having an amino acid sequence set forth in SEQ ID NO: 178, or
(18) a light chain CDR1 having an amino acid sequence set forth in SEQ ID NO: 179, a light chain CDR2 having an amino acid sequence set forth in SEQ ID NO: 180, and a light chain CDR3 having an amino acid sequence set forth in SEQ ID NO: 181.

6: The complex according to any one of above 1 to 3, further comprising any of the heavy chain variable regions defined in above 4, and any of the light chain variable regions defined in above 5.

7: The complex according to any one of above 2 to 6, in which
the heavy chain variable region includes a heavy chain CDR1 having an amino acid sequence set forth in SEQ ID NO: 104, a heavy chain CDR2 having an amino acid sequence set forth in SEQ ID NO: 105, and a heavy chain CDR3 having an amino acid sequence set forth in SEQ ID NO: 106, and
the light chain variable region includes a light chain CDR1 having an amino acid sequence set forth in SEQ ID NO: 149, a light chain CDR2 having an amino acid sequence set forth in SEQ ID NO: 150, and a light chain CDR3 having an amino acid sequence set forth in SEQ ID NO: 151.

8: The complex according to any one of above 2 to 7, in which the heavy chain variable region includes a framework region 1 having an amino acid sequence set forth in SEQ ID NO: 32, a framework region 2 having an amino acid sequence set forth in SEQ ID NO: 40, a framework region 3 having an amino acid sequence set forth in SEQ ID NO: 46, and a framework region 4 having an amino acid sequence set forth in SEQ ID NO: 54.

9: The complex according to any one of above 2 to 8, in which the light chain variable region includes a framework region 1 having an amino acid sequence set forth in SEQ ID NO: 57, a framework region 2 having an amino acid sequence set forth in SEQ ID NO: 72, a framework region 3 having an amino acid sequence set forth in SEQ ID NO: 79, and a framework region 4 having an amino acid sequence set forth in SEQ ID NO: 86.

10: The complex according to any one of above 1 to 9, in which
the heavy chain variable region has an amino acid sequence selected from the group consisting of SEQ ID NOS: 1 to 6, 8, and 9, and
the light chain variable region has an amino acid sequence selected from the group consisting of SEQ ID NOS: 10, 12 to 15, 17 to 21, and 29 to 31.

11: The complex according to any one of above 1 to 10, in which the heavy chain variable region has an amino acid sequence set forth in SEQ ID NO: 6, and the light chain variable region has an amino acid sequence set forth in SEQ ID NO: 21.

12: The complex according to any one of above 1 to 11, in which the radionuclide is a metal radionuclide,
the antibody and the metal radionuclide are linked via a linker,
the linker has a chelate site, and
the metal radionuclide chelated to the chelate site.

13: The complex according to above 12, in which
the linker has a peptide having an amino acid sequence consisting of 13 to 17 amino acid residues represented by Formula I below:

(X₁₋₃)-C-(X₂)—H-(Xaa1)-G-(Xaa2)-L-V-W-C-(X₁₋₃) (I)
{wherein, each X is independently any amino acid residue other than cysteine,
Xaa1 is a lysine residue, a cysteine residue, an aspartic acid residue, a glutamic acid residue, 2-aminosuberic acid, or diaminopropionic acid, and
Xaa2 is a glutamic acid residue, a glutamine residue, or an asparagine residue},
and a chelate site that chelates the radionuclide linked to the peptide, and
the peptide is cross-linked with the antibody.

14: The complex according to above 13, in which the number of bonds of the linker to the antibody is 1.

15: The complex according to any one of above 1 to 14, in which the radionuclide is a diagnostic metal radionuclide.

16: The complex according to any one of above 1 to 14, in which the radionuclide is a therapeutic metal radionuclide.

17: A composition containing the complex according to any one of above 1 to 16.

18: A composition containing the complex according to any one of above 1 to 16 and a pharmaceutically acceptable carrier, excipient or additive.

19: The complex according to any one of above 1 to 16, in which the radionuclide is ²²⁵Ac.

20: The complex according to any one of above 1 to 16, in which the radionuclide is ⁸⁹Zr.

21: The complex according to any one of above 1 to 16, 19, and 20, in which the chelate site includes DOTA or DOTA-GA.

22: The complex according to any one of above 1 to 16, 19, and 20, in which the chelate site includes DOTA-GA.

23: The complex according to above 13 or 14, in which the peptide has the amino acid sequence of SEQ ID NO: 186 (wherein X is a lysine residue).

24: The complex according to above 13 or 14, in which the chelate site includes DOTA or DOTA-GA, the metal radionuclide is ²²⁵Ac, and the peptide has the amino acid sequence of SEQ ID NO: 186 (wherein X is a lysine residue).

25: The complex according to above 13 or 14, in which the chelate site includes DOTA or DOTA-GA, the metal radionuclide is ⁸⁹Zr, and the peptide has the amino acid sequence of SEQ ID NO: 186 (wherein X is a lysine residue).

26: The complex according to any one of above 19 to 25, in which the antibody includes any of the heavy chain variable regions defined in above 4 and any of the light chain variable regions defined in above 5.

27: The antibody according to above 26, in which the antibody includes a heavy chain variable region having an amino acid sequence set forth in SEQ ID NO: 6 and a light chain variable region having an amino acid sequence set forth in SEQ ID NO: 21.

28: A complex having a structure of Formula (X) below:

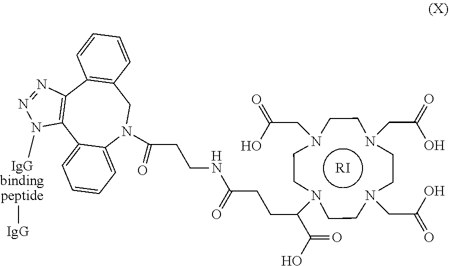

{wherein,
RI represents a metal radionuclide chelated to a chelate site,
the IgG-binding peptide is cross-linked with a crosslinkage agent, and
the IgG represents a humanized IgG antibody capable of binding to a human HEG1 protein expressed in mesothelioma cells}.

29: The complex according to above 28, in which the IgG-binding peptide has the amino acid sequence of SEQ ID NO: 186 (wherein X is a lysine residue).

30: The complex according to above 28, in which the IgG antibody contains any of the heavy chain variable regions defined in above 4 and any of the light chain variable regions defined in above 5.

31: The complex according to above 29, in which the IgG antibody contains any of the heavy chain variable regions defined in above 4 and any of the light chain variable regions defined in above 5.

32: The complex according to above 30, in which the IgG antibody contains a heavy chain variable region having an amino acid sequence set forth in SEQ ID NO: 6 and a light chain variable region having an amino acid sequence set forth in SEQ ID NO: 21.

33: The complex according to above 31, in which the IgG antibody contains a heavy chain variable region having an amino acid sequence set forth in SEQ ID NO: 6 and a light chain variable region having an amino acid sequence set forth in SEQ ID NO: 21.

34: The complex according to any one of above 30 to 34, in which the antibody includes
a heavy chain variable region including a framework region 1 having the amino acid sequence set forth in SEQ ID NO: 32, a framework region 2 having the amino acid sequence set forth in SEQ ID NO: 40, a framework region 3 having the amino acid sequence set forth in SEQ ID NO: 46, and a framework region 4 having the amino acid sequence set forth in SEQ ID NO: 54; and/or
a light chain variable region including a framework region 1 having the amino acid sequence set forth in SEQ ID NO: 57, a framework region 2 having the amino acid sequence set forth in SEQ ID NO: 72, a framework region 3 having the amino acid sequence set forth in SEQ ID NO: 79, and a framework region 4 having the amino acid sequence set forth in SEQ ID NO: 86.

35: The complex according to above 34, in which the antibody includes
a heavy chain variable region including a framework region 1 having the amino acid sequence set forth in SEQ ID NO: 33, a framework region 2 having the amino acid sequence set forth in SEQ ID NO: 40, a framework region 3 having the amino acid sequence set forth in SEQ ID NO: 46, and a framework region 4 having the amino acid sequence set forth in SEQ ID NO: 54, wherein in SEQ ID NO: 46, X1 is A, X3 is T, X5 is E, X6 is R, X7 is T, and in SEQ ID NO: 54, X1 is V, X3 is T, X4 is Q or E, and X5 is P.

36: The complex according to above 34, in which the antibody includes
a light chain variable region including a framework region 1 having the amino acid sequence set forth in SEQ ID NO: 61, a framework region 2 having the amino acid sequence set forth in SEQ ID NO: 74, a framework region 3 having the amino acid sequence set forth in SEQ ID NO: 81, and a framework region 4 having the amino acid sequence set forth in SEQ ID NO: 88.

37: The complex according to above 35, in which the antibody includes
a light chain variable region including a framework region 1 having the amino acid sequence set forth in SEQ ID NO: 61, a framework region 2 having the amino acid sequence set forth in SEQ ID NO: 74, a framework region 3 having the amino acid sequence set forth in SEQ ID NO: 81, and a framework region 4 having the amino acid sequence set forth in SEQ ID NO: 88.

38: The complex according to any one of above 28 to 37, in which the antibody includes one heavy chain variable region selected from the group consisting of SEQ ID NOS: 1 to 6, 8 and 9 and one light chain variable region selected from the group consisting of SEQ ID NOS: 10, 12 to 15, 17 to 21, 23 to 25, and 29 to 31.

39: The complex according to any one of above 28 to 38, in which the antibody includes one heavy chain variable region selected from the group consisting of SEQ ID NOs: 2, 4 to 6, 8, and 9 and one light chain variable region selected from the group consisting of SEQ ID NOS: 14, 15, 17 to 21, 23 to 25, and 29 to 31.

40: The complex according to any of above 28 to 39, including a heavy chain variable region set forth in SEQ ID NO: 6 and a light chain variable region set forth in SEQ ID NO: 21.

28B:

A complex having a structure of Formula (XI) below:

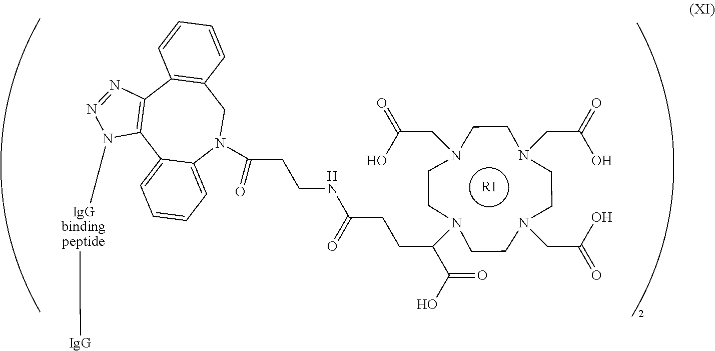

{wherein,
RI represents a metal radionuclide chelated to a chelate site,
the IgG-binding peptide is cross-linked with a crosslinkage agent, and
the IgG represents a humanized IgG antibody capable of binding to a human HEG1 protein expressed in mesothelioma cells}.

29B: The complex according to above 28B, in which the IgG-binding peptide has the amino acid sequence of SEQ ID NO: 186 (wherein X is a lysine residue).

30B: The complex according to above 28B, in which the IgG antibody contains any of the heavy chain variable regions defined in above 4 and any of the light chain variable regions defined in above 5.

31B: The complex according to above 29B, in which the IgG antibody contains any of the heavy chain variable regions defined in above 4 and any of the light chain variable regions defined in above 5.

32B: The complex according to above 30B, in which the IgG antibody contains a heavy chain variable region having an amino acid sequence set forth in SEQ ID NO: 6 and a light chain variable region having an amino acid sequence set forth in SEQ ID NO: 21.

33B: The complex according to above 31B, in which the IgG antibody contains a heavy chain variable region having an amino acid sequence set forth in SEQ ID NO: 6 and a light chain variable region having an amino acid sequence set forth in SEQ ID NO: 21.

34B: The complex according to any one of above 30B to 34B, in which the antibody includes
a heavy chain variable region including a framework region 1 having the amino acid sequence set forth in SEQ ID NO: 32, a framework region 2 having the amino acid sequence set forth in SEQ ID NO: 40, a framework region 3 having the amino acid sequence set forth in SEQ ID NO: 46, and a framework region 4 having the amino acid sequence set forth in SEQ ID NO: 54; and/or
a light chain variable region including a framework region 1 having the amino acid sequence set forth in SEQ ID NO: 57, a framework region 2 having the amino acid sequence set forth in SEQ ID NO: 72, a framework region 3 having the amino acid sequence set forth in SEQ ID NO: 79, and a framework region 4 having the amino acid sequence set forth in SEQ ID NO: 86.

35B: The complex according to above 34B, in which the antibody includes
a heavy chain variable region including a framework region 1 having the amino acid sequence set forth in SEQ ID NO: 33, a framework region 2 having the amino acid sequence set forth in SEQ ID NO: 40, a framework region 3 having the amino acid sequence set forth in SEQ ID NO: 46, and a framework region 4 having the amino acid sequence set forth in SEQ ID NO: 54, wherein in SEQ ID NO: 46, X1 is A, X3 is T, X5 is E, X6 is R, X7 is T, and in SEQ ID NO: 54, X1 is V, X3 is T, X4 is Q or E, and X5 is P.

36B: The complex according to above 34B, in which the antibody includes
a light chain variable region including a framework region 1 having the amino acid sequence set forth in SEQ ID NO: 61, a framework region 2 having the amino acid sequence set forth in SEQ ID NO: 74, a framework region 3 having the amino acid sequence set forth in SEQ ID NO: 81, and a framework region 4 having the amino acid sequence set forth in SEQ ID NO: 88.

37B: The complex according to above 35B, in which the antibody includes
a light chain variable region including a framework region 1 having the amino acid sequence set forth in SEQ ID NO: 61, a framework region 2 having the amino acid sequence set forth in SEQ ID NO: 74, a framework region 3 having the amino acid sequence set forth in SEQ ID NO: 81, and a framework region 4 having the amino acid sequence set forth in SEQ ID NO: 88.

38B: The complex according to any one of above 28 to 37, in which the antibody includes one heavy chain variable region selected from the group consisting of SEQ ID NOs: 1 to 6, 8 and 9 and one light chain variable region selected from the group consisting of SEQ ID NOs: 10, 12 to 15, 17 to 21, 23 to 25, and 29 to 31.

39B: The complex according to any one of above 28 to 38, in which the antibody includes one heavy chain variable region selected from the group consisting of SEQ ID NOS: 2, 4 to 6, 8, and 9 and one light chain variable region selected from the group consisting of SEQ ID NOS: 14, 15, 17 to 21, 23 to 25, and 29 to 31.

40B: The complex according to any of above 28B to 39B, including a heavy chain variable region set forth in SEQ ID NO: 6 and a light chain variable region set forth in SEQ ID NO: 21.

41: A method for detecting mesothelioma in a subject, including
injecting to the subject an effective amount of the complex according to any one of above 1 to 16, 28 to 40, and 28B to 40B, and
subsequently measuring the level or presence of radioactivity derived from the radionuclide in the subject.

42: A method for inferring a distribution status of mesothelioma or a distribution status and/or pharmacokinetics of a complex in a subject, the method including
injecting to the subject an effective amount of the complex according to any one of above 1 to 16, 28 to 40, and 28B to 40B, and
subsequently measuring the level or presence of radioactivity derived from the radionuclide in the subject.

43: A method for determining whether a subject has or is likely to have mesothelioma, the method including
injecting to the subject an effective amount of the complex according to any one of above 1 to 16, 28 to 40, and 28B to 40B, and
subsequently measuring the level or presence of radioactivity derived from the radionuclide in the subject.

44: The complex according to any one of above 1 to 16, 28 to 40, and 28B to 40B for use in the above-described method.

45: The method according to above 41, in which in the complex according to any one of above 1 to 16, 28 to 40, and 28B to 40B, the radionuclide is a diagnostic metal radionuclide.

44: The method according to above 42, in which in the complex according to any one of above 1 to 16, 28 to 40, and 28B to 40B, the radionuclide is a diagnostic metal radionuclide.

46: The method according to above 43, in which in the complex according to any one of above 1 to 16, 28 to 40, and 28B to 40B, the radionuclide is a diagnostic metal radionuclide.

47: The method according to above 44, in which in the complex according to any one of above 1 to 16, 28 to 40, and 28B to 40B, the radionuclide is a diagnostic metal radionuclide.

48: The complex according to any one of above 1 to 16, 28 to 40, and 28B to 40B for use in the method according to above 41.

49: The complex according to any one of above 1 to 16, 28 to 40, and 28B to 40B for use in the method according to above 42.

50: The complex according to any one of above 1 to 16, 28 to 40, and 28B to 40B for use in the method according to above 43.

51: The complex according to any one of above 1 to 16, 28 to 40, and 28B to 40B for use in the method according to above 44.

52: A composition containing the complex according to any one of above 1 to 16, 28 to 40, and 28B to 40B, for use in the method according to above 41.

53: A composition containing the complex according to any one of above 1 to 16, 28 to 40, and 28B to 40B, for use in the method according to above 42.

54: A composition containing the complex according to any one of above 1 to 16, 28 to 40, and 28B to 40B, for use in the method according to above 43.

55: A composition containing the complex according to any one of above 1 to 16, 28 to 40, and 28B to 40B, for use in the method according to above 44.

56: A method for treating mesothelioma in a subject, including
injecting to the subject an effective amount of the complex according to any one of above 1 to 16, 28 to 40, and 28B to 40B.

57: The method according to above 56, in which the subject is a subject determined to have mesothelioma by any one of the methods of above 41 to 44.

58: The method according to above 56, further including performing any one of the methods of above 41 to 44.

59: The method according to above 56, in which the radionuclide in the complex injected in the treatment is a therapeutic metal radionuclide.

60: The method according to above 57, in which the radionuclide in the complex injected in the treatment is a therapeutic metal radionuclide and the radionuclide in the complex injected in the detection, estimation, or determination is a diagnostic metal radionuclide.

61: The method according to above 58, in which the radionuclide in the complex injected in the treatment is a therapeutic metal radionuclide and the radionuclide in the complex injected in the detection, estimation, or determination is a diagnostic metal radionuclide.

62: The method according to any one of above 59 to 61, in which the metal radionuclide in the complex injected in the treatment are an α-ray emitting nuclide and the metal radionuclide in the complex injected in the detection, estimation or determination is a β- or γ-ray emitting nuclides.

63: The method according to any one of above 59 to 62, in which the metal radionuclide in the complex injected in the treatment is ²²⁵Ac and/or the metal radionuclide in the complex injected in the detection, estimation, or determination is ⁸⁹Zr.

64: A pharmaceutical composition containing an effective amount of the complex according to any one of above 1 to 16, 28 to 40, and 28B to 40B for use in the method according to any one of above 56 to 63.

65: A use of the complex according to any one of above 1 to 16, 28 to 40, and 28B to 40B in the production of the composition according to any one of above 52 to 55.

66: A use of the complex according to any one of above 1 to 16, 28 to 40, and 28B to 40B in the production of the composition according to above 64.

67: A composition containing the monovalent complex according to any one of above 28 to 40 and the divalent complex according to any one of above 28B to 40B, in which a molar ratio of the monovalent complex and the divalent complex (monovalent complex/divalent complex) is 2 or more, 3 or more, 4 or more, or 5 or more.

68: A composition containing the monovalent complex according to any one of above 28 to 40 and the divalent complex according to any one of above 28B to 40B, in which a molar ratio of the monovalent complex and the divalent complex (monovalent complex/divalent complex) is ½ or less, ⅓ or less, ¼ or less, or ⅕ or less.

69: A method for determining or estimating a therapeutic effect of a cancer therapy in a subject, the method including
injecting the complex according to any one of above 1 to 16, 28 to 40, and 28B to 40B to the subject before and after the cancer therapy, in which the radionuclide is a diagnostic metal radionuclide,
measuring radioactivity derived from the radionuclide in the body of the subject before and after cancer therapy, and obtaining the level and/or distribution of radioactivity before and after cancer therapy, respectively, and
comparing the level and/or distribution of radioactivity between before and after cancer therapy.

70: The method according to above 69, in which the cancer therapy is cancer therapy by injecting the complex according to any one of above 1 to 16, 28 to 40, and 28B to 40B, in which the radionuclide is a therapeutic metal radionuclide, to the subject.

71: The complex according to any one of above 1 to 16, 28 to 40, and 28B to 40B for use in the above-described method.

72: A modified antibody in which an IgG antibody is modified with a linker, in which
the IgG antibody is a humanized antibody that can bind to a human HEG1 protein expressed in mesothelioma cells,
the linker contains a peptide having an amino acid sequence consisting of 13 to 17 amino acid residues represented by Formula I below:

(X₁₋₃)-C-(X₂)—H-(Xaa1)-G-(Xaa2)-L-V-W-C-(X₁₋₃) (I)
{wherein, each of X's is independently any amino acid residue other than cysteine,
Xaa1 is a lysine residue, a cysteine residue, an aspartic acid residue, a glutamic acid residue, 2-aminosuberic acid, or diaminopropionic acid, and
Xaa2 is a glutamic acid residue, a glutamine residue, or an asparagine residue}, and the peptide is cross-linked with the IgG antibody.

### EXAMPLES

The present invention will be described more specifically with reference to the following examples, but the scope of the present invention is not limited by these examples.

### Production Example 1: Production of Humanized Antibody

The humanized antibody was designed to have an amino acid sequence of various variable regions to which a signal sequence (heavy chain: MGWSSIILFLVATTTGVHS (SEQ ID NO: 229), light chain: MKLPVRLLVLMFWIPASIS (SEQ ID NO: 230)) derived from a mouse antibody is added, and an amino acid sequence of a constant region of a human IgG1. The nucleotide sequence encoding the designed humanized antibody was converted to be a codon usage suitable for expression in Chinese hamster ovary (CHO) cells. A Kozak sequence was added to the base sequence obtained by conversion and the start codon site of the signal sequence, and a stop codon was added to the C-terminal side of the constant region. Furthermore, restriction enzyme sites were added upstream of the Kozak sequence and downstream of the stop codon. The above sequences obtained by ligating each of the mammalian cell expression plasmids (pcDNA 3.1) treated with a restriction enzyme were incorporated. Each of the obtained H chain expression plasmids and each of the L chain expression plasmids were expressed by ExpiCHO Expression System (ThermoFisher Scientific) or expressed in RK 13 cells to form a combination of one H chain and one L chain, and the plasmids were recovered from the culture supernatant by HiTrap protein G (1 mL) (Cytiba), eluted with 0.1 M glycine buffer (pH 2.8), and purified by dialysis against PBS.

The humanized antibody was obtained by the method described above. The antibody activity of each obtained antibody was measured by ELISA using 7.62 EGF as an antigen. The 7.62 EGF is a secreted protein in which an epitope region of an antibody is linked to the N-terminal side of the EGF domain region of HEG1 and a His tag is linked to the C-terminal side, and when produced in 293 H cells, sialylated sugar chain-modified EGF is recognized by the antibody.

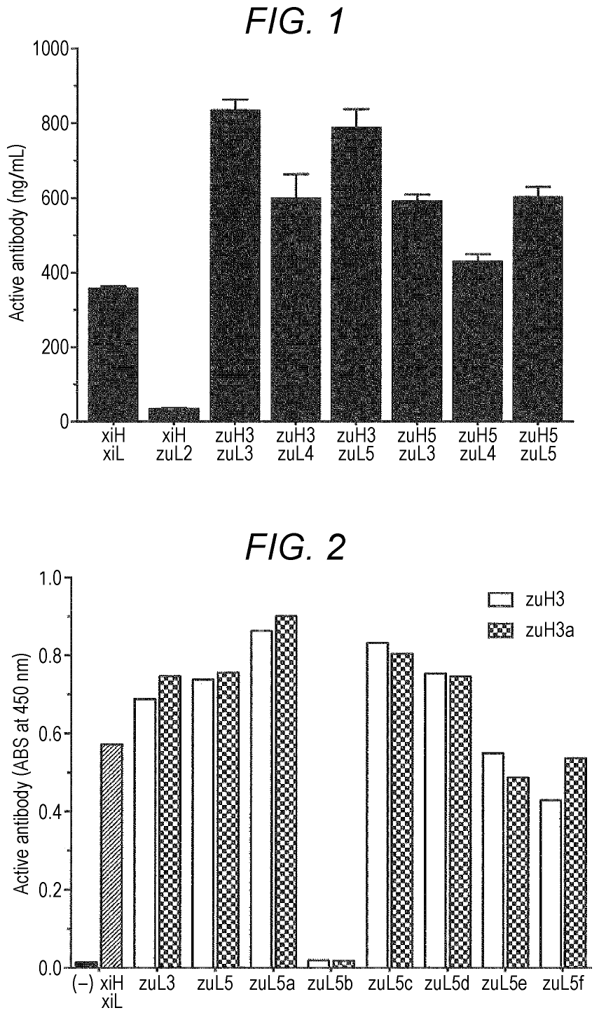

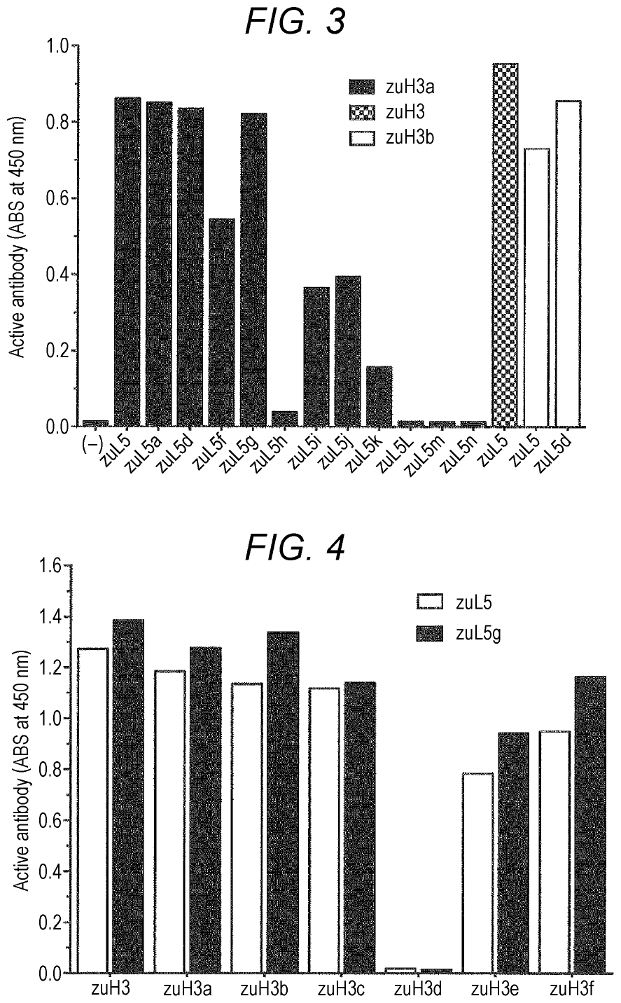

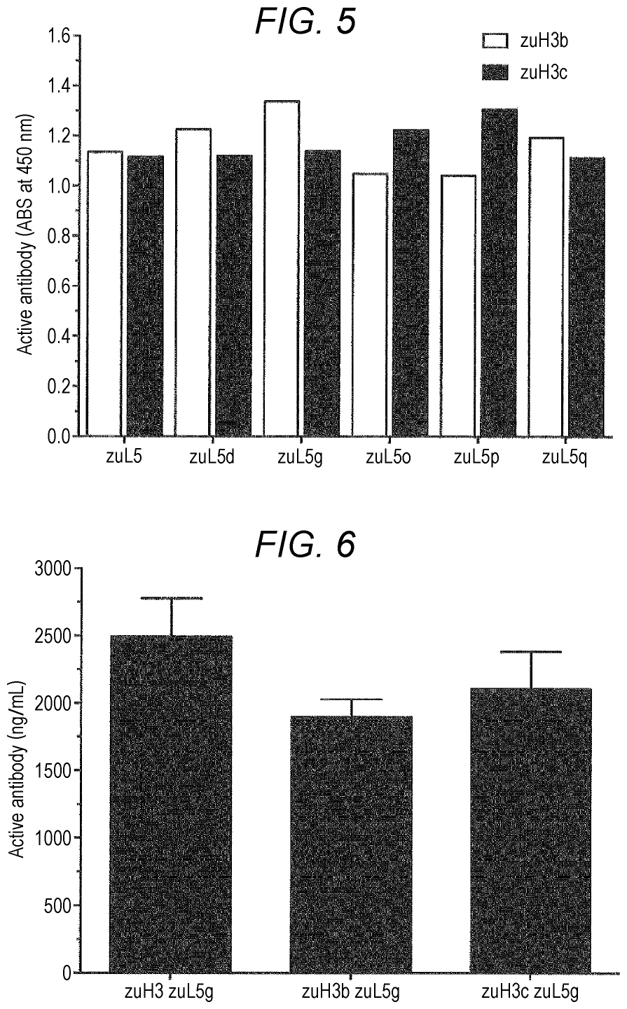

The 7.62 EGF purified from the culture supernatant of homeostatic expressing cells was adsorbed to an ELISA plate and blocked with 1% BSA. A culture supernatant of RK 13 cells into which a humanized antibody gene had been introduced was added to the plate, and the mixture was reacted at room temperature for 3 hours. After washing with 20 mM Tris buffered saline containing 0.1% Tween 20, a secondary antibody (horseradish peroxidase-labeled goat anti-human IgG, Fc gamma fragment specific) was added thereto, and the mixture was reacted for 90 minutes. After washing with 20 mM Tris buffered saline containing 0.1% Tween 20, 1-Step Ultra TMB-ELISA Substrate Solution (Thermo Fisher Scientific) was added. The reaction was stopped with 2 M sulfuric acid, and the absorbance at 450 nm was measured. The results are illustrated in FIGS. 1 to 6. The antigen binding activity was observed with a combination of “the antibodies containing the combinations of the amino acid sequence shown in Table 1 as a heavy chain and the amino acid sequence shown in Table 2 as a light chain”

| Clone name | Heavy chain amino acid sequence |
|---|---|
| zuH3 (SEQ ID NO: | QVQLVQSGAEVKKPGASVKLSCKASGFSFTTYWITWVR |
| 2) | QAPGQGLEWIGMIHPSDSETRLSQTFKDKATLTADKST |
| | NTAYMELSSLRSEDTAVYYCARDFDVWGQGTLVTVSS |
| zuH3a (SEQ ID | QVQLVQSGAEVKKPGASVKLSCKASGFSFTTYWITWVR |
| NO: 4) | QAPGQGLEWIGMIHPSDAETRLSQTFKDKATLTADKST |
| | NTAYMELSSLRSEDTAVYYCARDFDVWGQGILVTVSS |
| zuH3b (SEQ ID | QVQLVQSGAEVKKPGASVKVSCKASGFSFTTYNITWVR |
| NO: 5) | QAPGQGLEWIGMIHPSDAETRLSQTFKDKATLTADKST |
| | NTAYMELSSLRSEDTAVYYCARDFDVWGQGTLVTVSS |
| zuH3c (SEQ ID | QVQLVQSGAEVKKPGASVKVSCKASGFSFTTYWITWVR |
| NO: 6) | QAPGQGLEWIGMIHPSDAETRLSQTFKDKATLTADKST |
| | STAYMELSSLRSEDTAVYYCARDFDVWGQGTLVTVSS |
| zuH3e (SEQ ID | QVQLVQSGAEVKKPGASVKVSCKASGFHFTTYWITWVR |
| NO: 8) | QAPGQGLEWIGMIHPSDAETRLSQTFKDKATLTADKST |
| | NTAYMELSSLRSEDTAVYYCARDFDVWGQGTLVTVSS |
| zuH3f (SEQ ID | QVQLVQSGAEVKKPGASVKVSCKASGFSFRTYWITWVR |
| NO: 9) | QAPGQGLEWIGMIHPSDAETRLSQTFKDKATLTADKST |
| | NTAYMELSSLRSEDTAVYYCARDFDVWGQGTLVTVSS |
| zuH5 (SEQ ID NO: | QVQLVQSGAEVKKPGASVKLSCKASGFSFTTYWITWVR |
| 3) | QAPGQGLEWIGMIHPSDSETRLSQTFKDKATLTADEST |
| | NTAYMELSSLRSEDTAVYYCARDFDVNGQGTLVTVSS |
| Clone name | Light chain amino acid sequence |
| zuL3 (SEQ ID NO: | DVVMTQTPLSLPVTLGQPASISCRSSQNIVHNNGNTYL |
| 12) | GWYLQKPGQSPQLLIFKVSNRFSGVPDRFSGSGSGTDF |
| | TLKISRVEAEDVGVYYCFQGSHIPRTFGQGTRLEIK |
| zuL4 (SEQ ID NO: | DVVMTQTPLSLPVTPGEPASISCRSSQNIVHNNGNTYL |
| 13) | GWYLQKPGQSPQLLIFKVSNRFSGVPDRFSGSGSGTDF |
| | TLKISRVEAEDVGVYYCFQGSHIPRTFGQGTRLEIK |
| zuL5 (SEQ ID NO: | DVVMTQSPLSLPVTLGQPASISCRSSQNIVHNNGNTYL |
| 14) | GWYLQKPGQSPQLLIFKVSNRFSGVPDRFSGSGSGTDF |
| | TIKISRVEAEDVGVYYCFQGSHIPRTFGQGTRLEIK |
| zuL5a (SEQ ID NO: | DVVMTQSPLSLPVTLGQPASISCRSSQNIVHQNGNTYL |
| 15) | GWYLQKPGQSPQLLIFKVSNRFSGVPDRFSGSGSGTDF |
| | TLKISRVEAEDVGVYYCFQGSHIPRTFGQGTRLEIK |
| zuL5c (SEQ ID NO: | DVVMTQSPLSLPVTLGQPASISCRSSQNIVHNNGQTYL |
| 17) | GWYLQKPGQSPQLLIFKVSNRFSGVPDRFSGSGSGTDF |
| | TLKISRVEAEDVGVYYCFQGSHIPRTFGQGIRLEIK |
| zuL5d (SEQ ID NO: | DVVMTQSPLSLPVTLGQPASISCRSSQNIVINNGNTYL |
| 18) | GWYLQKPGQSPQLLIFRVSNRFSGVPDRESGSGSGTDF |
| | TLKISRVEAEDVGVYYCFQGSHIPRTFGQGTRLEIK |
| zuL5e (SEQ ID NO: | DVVMTQSPLSLPVTLGQPASISCRSSQNIVHQNAQTYL |
| 19) | GWYLQKPGQSPQLLIFKVSNRFSGVPDRFSGSGSGTDF |
| | TLKISRVEAEDVGVYYCFQGSHIPRTFGQGTRLEIK |
| zuL5f (SEQ ID NO: | DVVMTQSPLSLPVTLGQPASISCRSSQNIVHQNAQTYL |
| 20) | GWYLQKPGQSPQLLIFRVSNRFSGVPDRFSGSGSGTDF |
| | TLKISRVEAEDVGVYYCFQGSHIPRTFGQGTRLEIK |
| zuL5g (SEQ ID NO: | DVVMTQSPLSLPVTLGQPASISCRSSQNIVHQNGQTYL |
| 21) | GWYLQKPGQSPQLLIFRVSNRFSGVPDRFSGSGSGTDF |
| | TLKISRVEAEDVGVYYCFQGSHIPRTFGQGTRLEIK |
| zuL5i (SEQ ID NO: | DVVMTQSPLSLPVTLGQPASISCRSSQNIVHQTGQTYL |
| 23) | GWYLQKPGQSPQLLIFRVSNRFSGVPDRFSGSGSGTDF |
| | TLKISRVEAEDVGVYYCFQGSHIPRTFGQGTRLEIK |
| zuL5j (SEQ ID NO: | DVVMTQSPLSLPVILGQPASISCRSSQNIVHQNRQTYL |
| 24) | GWYLQKPGQSPQLLIFRVSNRFSGVPDRFSGSGSGTDF |
| | TLKISRVEAEDVGVYYCFQGSHIPRTFGQGTRLEIK |
| zuL5k (SEQ ID NO: | DVVMTQSPLSLPVTLGQPASISCRSSQNIVHQNAGTYL |
| 25) | GWYLQKPGQSPQLLIFRVSNRFSGVPDREFSGSGSGTDF |
| | TLKISRVEAEDVGVYYCFQGSHIPRTFGQGERLEIK |
| zuL5o (SEQ ID NO: | DVVMTQSPLSLPVTLGQPASISCRSSQNIVHQNGQTYL |
| 29) | GWYLQKPGQSPQLLIFRVSNRFSGVPDRFSGSGSGTDF |
| | TLKISRVEAEDVGVYYCFQGSHNPRTFGQGTRLEIK |
| zuL5p (SEQ ID NO: | DVVMTQSPLSLPVTLGQPASISCRSSQNIVHQNGQTYL |
| 30) | GWYLQKPGQSPQLLIFRVSNRESGVPDRFSGSGSGTDF |
| | TLKISRVEAEDVGVYYCFQGSHHPRTFGQGTRLEIK |
| zuL5q (SEQ ID NO: | DVVMTQSPLSLPVTLGQPASISCRSSQNIVHQNGQTYL |
| 31) | GWYLQKPGQSPQLLIFRVSNRFLGVPDRFSGSGSGTDF |
| | TLKISRVEAEDVGVYYCFQGSHIPRTFGQGTRLEIK |

### Production Example 2: Production of Humanized SKM9-2 Using Different Signal Sequences

Among the antibodies obtained in Production Example 1, an antibody having high antigen binding activity and many human-derived sequences (heavy chain: zuH3c, light chain: zuL 5g) was used as a representative humanized SKM9-2, and the signal sequence was examined.

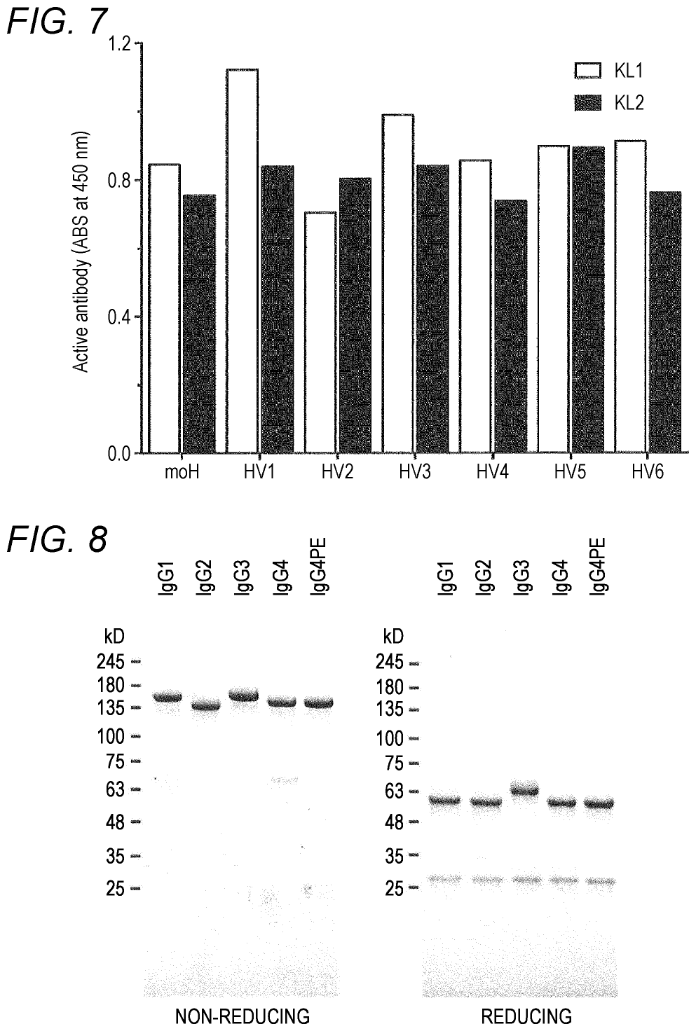

An antibody having zuH3c as the amino acid sequence of the heavy chain and zuL 5g as the amino acid sequence of the light chain was produced in the same manner as in Production Example 1 except that the signal sequences shown in Table 3 were used. The results of the antibody production amount are shown in FIG. 7. No large difference was observed in the production amount of the antibody even in a case where any signal sequence was used, but when HV1 (SEQ ID NO: 221) was used as the heavy chain signal sequence and KL1 (SEQ ID NO: 227) was used as the light chain signal sequence, the production amount of the antibody tended to be slightly large.

| Sequence name | Sequence | SEQ ID NOS |
|---|---|---|
| Heavy chain signal | MDWTWRILFLVAAATGAHS | SEQ ID NO: 221 |
| sequence HV1 | | |
| Heavy chain signal | MDILCSTLLLLTVPSWVLS | SEQ ID NO: 222 |
| sequence HV2 | | |
| Heavy chain signal | MEFGLSWVFLVALLRGVQC | SEQ ID NO: 223 |
| sequence HV3 | | |
| Heavy chain signal | MKHLWFFLLLVAAPRWVLS | SEQ ID NO: 224 |
| sequence HV4 | | |
| Heavy chain signal | MGSTAILALLLAVLQGVCA | SEQ ID NO: 225 |
| sequence HV5 | | |
| Heavy chain signal | MSVSFLIFLPVLGLPWGVLS | SEQ ID NO: 226 |
| sequence HV6 | | |
| Light chain signal | MDMRVPAQLLGLLLLWVPGARC | SEQ ID NO: 227 |
| sequence KL1 | | |
| Light chain signal | MRLPAQLLGLLMLWVPGSSG | SEQ ID NO: 228 |
| sequence KL2 | | |
| Mouse heavy chain | MGWSSIILFLVATTTGVHS | SEQ ID NO: 229 |
| signal sequence | | |
| Mouse light chain | MKLPVRLLVLMFWIPASIS | SEQ ID NO: 230 |
| signal sequence | | |

### Production Example 3: Production of Humanized Antibodies of Different Subtypes

A humanized antibody was produced in the same manner as in Production Example 1 except that as the heavy chain, one linked to the constant region (CH1-CH3) of human IgG using HV1 (SEQ ID NO: 221) as the signal sequence and zuH3c (SEQ ID NO: 6) as the variable region was used, and as the light chain, one linked to the constant region (CL) of human kappa chain using KL1 (SEQ ID NO: 227) as the signal sequence and zuL 5g as the variable region was used. The IgG4PE is a variant in which two amino acid residues in the vicinity of the hinge are changed (S228P and L235E).

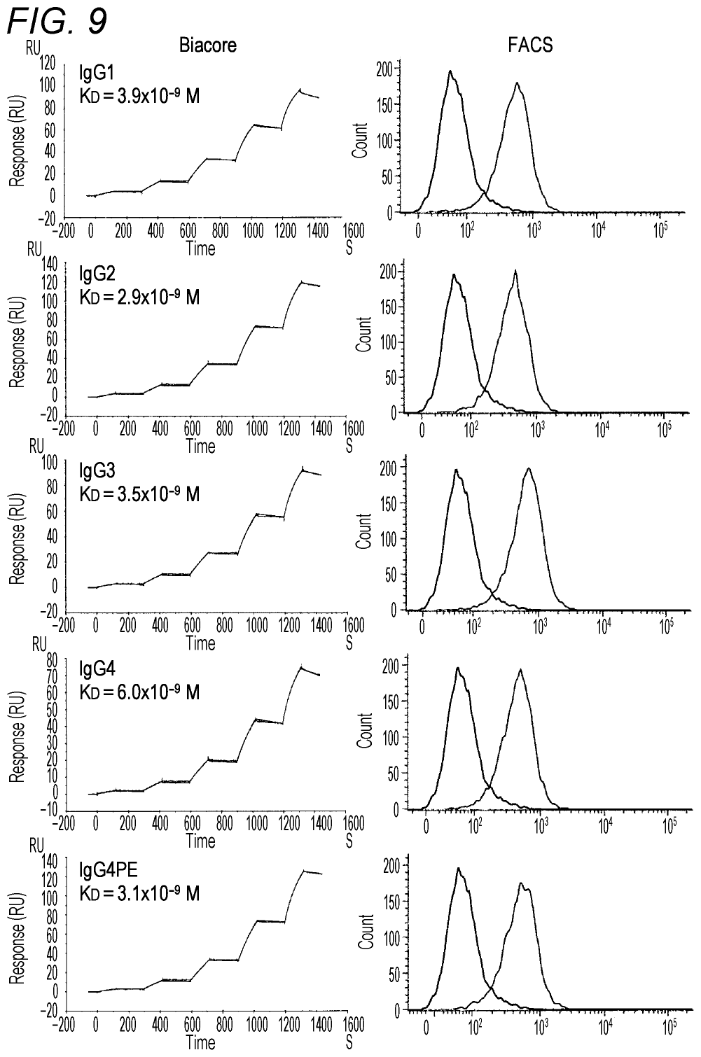

The results of SDS-PAGE of the obtained antibodies are illustrated in FIG. 8, and the results of analyzing the binding to the synthetic sugar peptide epitope represented by the following formula by Biacore X 100 and the results of analyzing the binding to mesothelioma cell line NCI-H226 by flow cytometry are illustrated in FIG. 9. Biacore used a His-tagged synthetic glycopeptide as ligand and an antibody as analyte. As the sensor chip, a sensor chip NTA bonded with Ni²⁺ was used. The affinity was calculated according to the protocol of Biacore X100. For flow cytometry, the purified antibody was used as a primary antibody at 10 μg/mL, and FITC-goat anti-human IgG F(ab)′2 was used as a secondary antibody at 25 μg/mL. The black line indicates no primary antibody (negative control), and the red line indicates addition of each primary antibody. No significant difference was observed between the antibodies for both Biacore and flow cytometry.

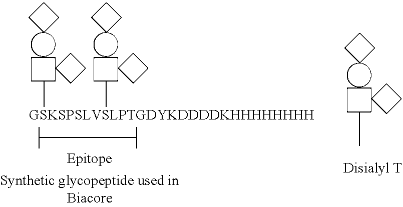

### Example 1: Production of Antibody-RI Conjugates Using ²²⁵Ac-Labeled DOTAGA-DBCO

### (1-1. Production of Antibody Modification Linker)

A peptide containing 17 amino acid residues represented by the following (P3) was obtained by the method described in WO2017/217347. The amino acid sequence of this peptide was the same as the sequence in which Xaa1 of SEQ ID NO: 186 was a lysine residue, and the side chain terminal amino group of the lysine residue was modified with the structure represented by R1. In addition, two cysteine residues are disulfide bonded to each other, and ethyl azide is bonded to the N-terminal side of the peptide as an atomic group containing an azide group as a second atomic group via diglycolic acid and a linker (L1) structure having eight PEGs.

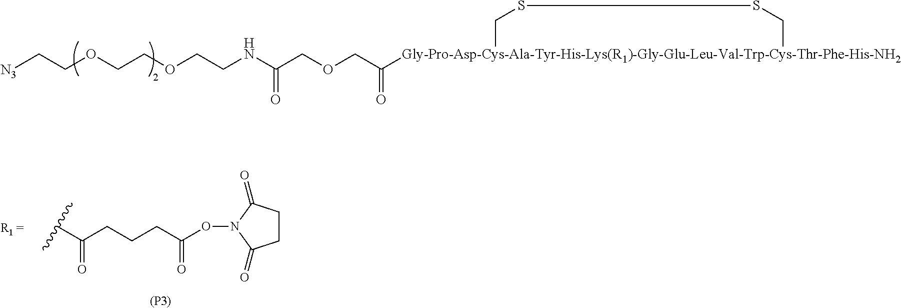

(wherein Gly represents glycine, Pro represents proline, Asp represents aspartic acid, Cys represents cysteine, Ala represents alanine, Tyr represents tyrosine, His represents histidine, Glu represents glutamic acid, Leu represents leucine, Val represents valine, Trp represents tryptophan, and Phe represents phenylalanine in Formula (P3))

### (1-2. Modification of Antibody by Antibody Modification Linker)

According to the method described in WO2017/217347, the peptide DSG (disuccinimidyl glutarate) described above was reacted. The obtained solution containing a DSG-modified peptide and the solution containing the antibody (IgG4PE) obtained in Production Example 3 were mixed in 0.02 mol/L acetic acid-sodium acetate buffer (pH 6.0), and reacted at room temperature for 60 minutes to obtain a solution containing a peptide-modified antibody. In this peptide-modified antibody, the Fc region of the antibody was site-specifically modified with the peptide described above.

### (1-3. Separation and Purification of Monovalent Antibody)

Next, the mixture was passed through an IgG-BP column to obtain a first antibody composition containing a relatively large amount of unlabeled antibody and monovalent antibody. The concentration of the monovalent antibody contained in the collected fraction was adjusted to 15 mg/mL with a 0.02 mol/L phosphate buffer (pH 6.0) containing 0.1 mol/L sodium chloride. A solution containing the obtained first antibody composition was subjected to a labeling process described later.

### (2. Complex Formation Process)

DOTAGA-DBCO represented by the following formula was produced on the basis of the method described in Bernhard et al. DOTAGA-Anhydride: AValuable Building Block for the Preparation of DOTA-Like Chelating Agents Chem. Eur. J. 2012, 18, 7834-7841. This chelating agent was dispersed in 0.1 mol/L sodium acetate buffer (pH 6.0) as a solvent to obtain a dispersion containing 1.7 mmol/L of the chelating agent. A reaction solution prepared by mixing 0.005 mL of this dispersion, 0.075 mL of a 0.1 mol/L sodium acetate buffer (pH 6.0), and 2.1 MBq of a ²²⁵Ac ion-containing solution (0.2 mol/L hydrochloric acid aqueous solution, radiation concentration: 420 MBq/mL, adjusted by Oak Ridge National Laboratory, liquid volume: 0.005 mL) as a radioactive metal source (calculated value of attenuation calculated from the radioactivity on the day of verification) was reacted under heating conditions to obtain a ²²⁵Ac complex solution. The molar ratio of the chelating agent and the radioactive metal ion was chelating agent: ²²⁵Ac ion=about 1900:1, the heating condition of the reaction solution was 70° C., and the heating time was 30 minutes.

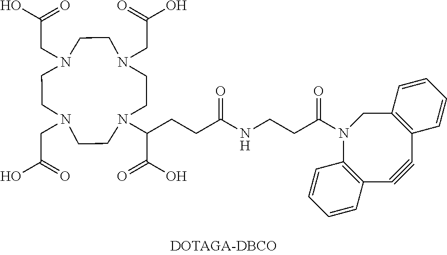 [SMILES: O=C(O)CN1CCN(CC(=O)O)CCN(C(CCC(=O)NCCC(=O)N2Cc3ccccc3C#Cc3ccccc32)C(=O)O)CCN(CC(=O)O)CC1]

The radiochemical purity of the obtained ²²⁵Ac complex was measured by the following method. That is, a part of the ²²⁵Ac complex solution was developed by thin layer chromatography (manufactured by Agilent Technologies, model number: SGI 0001, developing solvent: acetonitrile:a mixed solution of 0.1 mol/L EDTA solution (pH 5.0) (volume ratio: 1:1)), and then measured by a radio-γ-TLC analyzer (manufactured by raytest GmbH, MODEL GITA Star). The percentage of the radioactivity (count) of the peak detected near the origin relative to the total radioactivity (count) detected was taken as the radioactive purity (%) of the ²²⁵Ac complex. As a result, the radiochemical purity of the ²²⁵Ac complex was 86.3%. The obtained ²²⁵Ac complex solution was used as it was in the labeling process.

A solution containing the peptide-modified antibody (monovalent antibody) obtained in Process (1) above was added to the solution of the unpurified ²²⁵Ac complex obtained through Process (2) above, and a click reaction was performed at 37° C. for 2 hours to obtain a ²²⁵Ac complex labeled antibody. The molar ratio of DBCO groups to azide groups during the reaction was about 1:1.2, respectively. The reaction rate (%) of the unpurified ²²⁵Ac complex labeled antibody was 82%. Here, the reaction rate (%) means the radiochemical purity (%) of the ²²⁵Ac complex labeled antibody with respect to the labeling rate (%) in the complex formation process, and the labeling rate (%) means the radioactivity (%) of the ²²⁵Ac complex with respect to the charged radioactivity. Furthermore, a solution of the ²²⁵Ac complex labeled antibody obtained by reacting at 37° C. for 2 hours was purified using an ultrafiltration filter (manufactured by Merck KGAA, model number: UFC 505096). The radiochemical purity (RCP) of the ²²⁵Ac complex labeled antibody after purification was 97%, and the radiochemical yield (RCY) was 73%.

The method for measuring the radiochemical purity and radiochemical yield of the ²²⁵Ac complex labeled antibody was as follows. That is, thin layer chromatography (manufactured by Agilent Technologies, model number: SGI 0001, developing solvent: acetonitrile:a mixed solution of 0.1 mol/L EDTA solution (pH 5.0) (volume ratio: 1:1)) was measured with a radio γ-TLC analyzer (manufactured by raytest GmbH, MODEL GITA Star), and the percentage of the radioactivity (count) of the peak detected in the vicinity of the origin with respect to the total radioactivity (count) detected was taken as the radiochemical purity (%). In addition, the percentage of radioactivity (in the same manner as described above, radioactivity calculated from counts measured with a γ-ray spectrometer) recovered after ultrafiltration purification relative to the total radioactivity (radioactivity calculated from counts measured with a γ-ray spectrometer (Ge semiconductor detector: GMX 10 P4-70 (manufactured by ORTEC LTD.), multi-channel analyzer: M7-000 (manufactured by SEIKO EG&G CO., LTD.), data processing: SpectrumNavigator: DS-P 300 (manufactured by SEIKO EG&G CO., LTD.), and Gamma Studio: DS-P 600 (manufactured by SEIKO EG&G CO., LTD.))) added at the start of the labeling process was defined as the radiochemical yield (%).

### Example 2: Production of Complex with Antibody of Present Disclosure Using ⁸⁹Zr-Labeled DOTAGA-DBCO

### (1. Complex Formation Process)

The DOTAGA-DBCO was dispersed in 0.156 mol/L sodium acetate buffer (pH 5.5) as a solvent to form a dispersion containing 0.3 mmol/L of a chelating agent. A reaction solution of 0.06 mL of this dispersion, 0.06 mL of 0.156 mol/L sodium acetate buffer (pH 5.5) containing 0.15 mol/L gentisic acid, and 338 MBq of a solution containing ⁸⁹Zr (⁸⁹Zr produced by the ⁸⁹Y(p,n)⁸⁹Zr method was adjusted to a 0.1 mol/L hydrochloric acid aqueous solution, radioactivity concentration 5.63 GBq/mL, liquid volume 0.06 mL) ions as a radioactive metal source was reacted under heating conditions to obtain an ⁸⁹Zr complex solution. The molar ratio of the chelating agent and the radioactive metal ion was the chelating agent: ⁸⁹Zr ion=about 80:1, the heating temperature of the reaction solution was 70° C., and the heating time was 1 hour.

The radiochemical purity of the obtained ⁸⁹Zr complex was measured in the same manner as when the radiochemical purity of the ²²⁵Ac complex was measured in Example 1. As a result, the radiochemical purity of the ⁸⁹Zr complex was 52.4%. The obtained ⁸⁹Zr complex solution was used as it was in the labeling process.

### (2. Labeling Process)

A solution containing a peptide-modified antibody (monovalent antibody) obtained in the same manner as in Example 1 was added to the unpurified ⁸⁹Zr complex solution obtained through Process (1) described above, and a click reaction was performed at 37° C. for 2 hours to obtain the ⁸⁹Zr complex labeled antibody. The molar ratio of DBCO to azide during the reaction was about 1:1.2, respectively. The reaction rate (%) of the unpurified ⁸⁹Zr complex labeled antibody was 71%.

Furthermore, a solution of the ⁸⁹Zr complex labeled antibody obtained by reacting at 37° C. for 2 hours was purified using an ultrafiltration filter (manufactured by Merck KGAA, model number: UFC 505096). The radiochemical purity (RCP) of the ⁸⁹Zr complex labeled antibody after purification was 81%, and the radiochemical yield (RCY) was 58%. The method for measuring the radiochemical purity and radiochemical yield of the ⁸⁹Zr complex labeled antibody was performed in the same manner as in Example 1.

### Example 3: Formulation Process

The ²²⁵Ac complex labeled antibody produced according to Example 1 and the ⁸⁹Zr complex labeled antibody produced according to Example 2 were each partially withdrawn in a 5 mL Eppendorf tube (LoBind, manufactured by Eppendorf Corporation) and diluted with a preservation buffer (0.02 mol/L sodium phosphate buffer (pH 6.0) containing 0.1 mol/L sodium chloride).

### Evaluation 1: Stability Evaluation of ²²⁵Ac Complex Labeled Antibody

The ²²⁵Ac complex labeled antibody obtained in Example 3 was stored at room temperature (24.5 to 25.5° C.) for 1 week, and the radiochemical purity and the ratio of aggregates were evaluated at each time point (0 day point, 1 day point, and 7 day point). In addition, the antigen binding activity to a tumor section is evaluated using the radioactive complex produced in the same manner. The ²²⁵Ac complex labeled antibody used in the following evaluation was produced according to the description of Example 1, and formulated according to the description of Example 3.

### Evaluation 1-1: Radiochemical Purity

The radiochemical purity was analyzed using thin layer chromatography (TLC). The conditions for TLC were the same as those used when the reaction rate was evaluated in Example 1. The results are shown in Table 4.

Radiochemical purity (%)

| | 0 day point | 1 day point | 7 day point |
|---|---|---|---|
| ²²⁵Ac complex | 99.96 | 99.83 | 97.35 |
| labeled antibody | | | |

In a case where the ²²⁵Ac complex labeled antibody was stored at room temperature for 7 days after the end of production, the radiochemical purity was maintained at 97% or more.

### Evaluation 1-2: Ratio of Aggregates

The ratio of aggregates contained in the ²²⁵Ac complex labeled antibody was confirmed by size exclusion chromatography (SEC) at each time point (0 day point, 1 day point, and 7 day point). The analysis was performed under the following conditions using a 2695 type separation module or an e2695 type separation module manufactured by Waters Corporation as a liquid chromatography apparatus and a 2489 type UV/Vis detector manufactured by Waters Corporation as a UV detector. Table 5 shows the ratio of each component in a case where the product is stored for 7 days after completion of production.

| | Main peak | Aggregate peak |
|---|---|---|
| | percentage (%) | percentage (%) |
| ²²⁵Ac complex labeled | 97.45 | 2.55 |
| antibody (Produced 0 | | |
| days after) | | |
| ²²⁵Ac complex labeled | 92.60 | 6.43 |
| antibody (Produced 1 | | |
| day after) | | |
| ²²⁵Ac complex labeled | 76.35 | 21.28 |
| antibody (Produced 7 | | |
| days after) | | |

### HPLC Conditions

Column: TOSOH TSKgel guard column SWXL (6 mm×4 cm), TOSOH TSKgel G 3000 SWXL (5 μm, 7.8×30 cm)×2 columns (series)
Column temperature: Constant temperature around 25° C.
Mobile phase: 0.1 mol/L phosphate buffer (pH 6.8) containing 0.2 mol/L arginine hydrochloride
Flow rate: 1.0 mL per minute
Area measurement range: 30 minutes
Detection wavelength: 280 nm

### Evaluation 1-3: Antigen Binding Activity of ²²⁵Ac Complex Labeled Antibody to Tumor Section

The antigen binding activity of the ²²⁵Ac complex labeled antibody, which was produced according to the description of Example 1 and formulated according to the description of Example 3, to tumor sections was confirmed by in vitro autoradiography (ARG) (0 days after production). Tumor-bearing mice were generated by subcutaneously injecting 5×10⁶ cells of HEG1-positive cells, NCI-H226 cells (purchased from American Type Culture Collection (hereinafter also referred to as “ATCC”)) and ACC-MESO4 cells (purchased from Cell Engineering Division, RIKEN BioResource Center (hereinafter also referred to as “RIKEN BRC”); see Cancer Sci 2006; 97:387-394), and HEG1-negative cells, A549 cells (purchased from The European Collection of Authenticated Cell Cultures (hereinafter also referred to as “ECACC”)), into the flanks of female BALB/c nu/nu mice (provided by Charles River Laboratories Japan, Inc.). Thereafter, the tumor was excised and embedded in Tissue-Tec O.C.T compound (manufactured by Sakura Finetek Japan Co., Ltd.) to prepare a frozen section. A ²²⁵Ac complex labeled antibody was added to phosphate buffer saline (pH 7.4) (hereinafter, also referred to as “PBS”) containing 1% bovine serum albumin to 1 kBq/mL, and NCI-H226 tumor sections, ACC-MESO4 tumor sections, and A549 tumor sections were immersed. The sections was brought into contact with an imaging plate (manufactured by FUJIFILM co., Ltd.) and then read by a scanner type image analyzer (Typhoon FLA 7000, manufactured by GE Healthcare Japan) to obtain an autoradiogram. Five regions of interest (ROI) were set in each tumor section of the obtained autoradiogram using Image Quant TL (manufactured by Cytiva), and the radioactivity count per unit area of each ROI was calculated. Using the average of the calculated values, the antigen binding activity of the ²²⁵Ac complex labeled antibody to each tumor section was evaluated. The results are shown in FIG. 11. The vertical axis of the graph represents the radioactivity count per unit area (Counts/pixel) of the ROI. Graphs represent mean±standard deviation for each group (n=2). For the statistically significant difference test, a parametric Tukey-type multiple comparison test was performed using Stat Preclinica (manufactured by Takumi Information Technology Inc.).

As illustrated in FIG. 11, regarding the binding of the antibody to the tumor sections, a statistically significant difference was observed between the HEG1-positive tumor sections (NCI-H226 and ACC-MESO4) and the HEG1-negative tumor section (A549) (p<0.01). From this, the radioactive complex produced according to the description of Example 1 and formulated according to the description of Example 3 bound only to HEG1-positive tumor (NCI-H226 and ACC-MESO4) sections and showed HEG1 selective binding.

### Evaluation 1-4: Antigen Binding Activity of ²²⁵Ac Complex Labeled Antibody to Living Cell

Using the cultured cells, the antigen binding activity of the radioactive complex produced according to the description of Example 1 and formulated according to the description of Example 3 was confirmed.

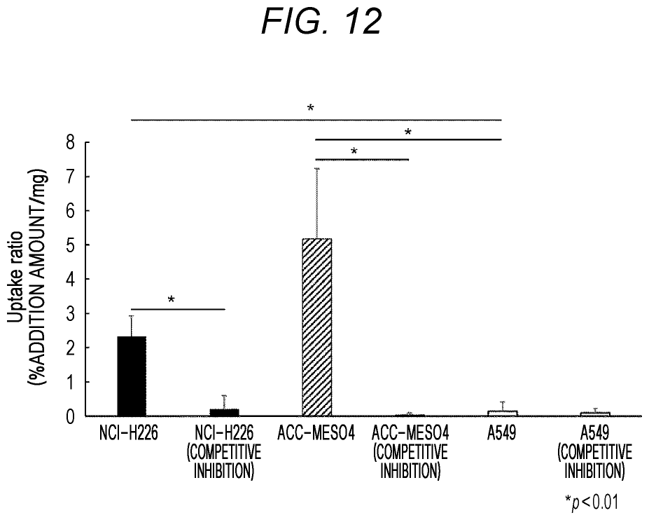

HEG1-positive NCI-H226 cells (purchased from ATCC), ACC-MESO4 cells (purchased from RIKEN BRC), and HEG1-negative A549 cells (purchased from ECACC) were suspended in RPMI1640 medium (manufactured by Gibco) (contains 10% fetal bovine serum (manufactured by BioWest), 1% penicillin-streptomycin (manufactured by Gibco)) or Ham's F-12K medium (manufactured by Gibco) (contains 10% fetal bovine serum (manufactured by ATCC), 1% penicillin-streptomycin (manufactured by Gibco)), and each cell was incubated in a 24-well plate (1.0×10⁵ cells/well) at 37° C. under 5% carbon dioxide and atmospheric pressure for 24 hours. After removing the medium, 0.4 mL of a medium containing a ²²⁵Ac complex labeled antibody (finally added radioactivity 2 kBq/well) was added to each well, and the plate was incubated at 37° C. for 1 hour under atmospheric conditions. Separately, in order to confirm the HEG1 specificity of the ²²⁵Ac complex labeled antibody, incubation was performed in the same condition except that unlabeled SKM9-2 (final concentration: 0.5 μM) was added as a competing compound for the ²²⁵Ac complex labeled antibody. Thereafter, each well was washed with 0.5 mL of PBS, and the cells were dissolved in 0.15 mL of a 2 N aqueous sodium hydroxide solution (manufactured by NACALAI TESQUE, INC.) at room temperature. Thereafter, 0.15 mL of ultrapure water (manufactured by Millipore) was added to each well to make the solution volume 0.3 mL, and 0.2 mL of the solution was fractionated, and radioactivity in the solution was measured with a gamma counter (2470 WIZARD² autogamma counter, manufactured by PerkinElmer Inc). The solution remaining in the well was dried completely and dissolved in 0.4 mL of ultrapure water, and then the absorbance at 562 nm was measured with a plate reader (SpectraMax i3x, manufactured by Molecular Devices, LLC.) using Pierce BCA Protein Assay Kit (manufactured by Thermo Fisher Scientific Inc.), and the protein concentration of each well was calculated. Among the radioactivity added to each well, the ratio of the radioactivity bound to the cell was corrected by the amount of protein in the well (defined as Uptake ratio (% addition amount/mg)). A parametric Tukey-type multiple comparison test was performed using Stat Preclinica (manufactured by Takumi Information Technology Inc.), and a statistical significant difference test was performed. The results are shown in FIG. 12. The vertical axis of the graph represents a value (uptake ratio (% addition amount/mg)) obtained by correcting the ratio of the radioactivity bound to the cell among the radioactivity added to each well by the amount of protein in the well. Graph plots and error bars represent mean±standard deviation of each group (n=4).

As illustrated in FIG. 12, regarding the binding of the antibody to living cells, a statistically significant difference was observed between the HEG1-positive NCI-H226 cell group or the ACC-MESO4 cell group and the HEG1-negative A549 cell group (p<0.01). In addition, the binding of the ²²⁵Ac complex labeled antibody to NCI-H226 cells and ACC-MESO4 cells in the presence of the competing compound (unlabeled antibody) was confirmed to have a statistically significant inhibitory effect as compared with that in the absence of the competing compound (p<0.01). On the other hand, regarding the binding of the ²²⁵Ac complex labeled antibody to A549 cells, no statistically significant difference at the 5% significance level was observed between the presence and absence of the competing compound, and no competitive inhibition was confirmed. From these, it was shown that the ²²⁵Ac complex labeling antibody has binding and specificity to the HEG1.

### Evaluation 1-5: Evaluation of Cell-Killing Effect of ²²⁵Ac Complex Labeled Antibody

Using the cultured cells, the cell-killing effect of the radioactive complex produced according to the description of Example 1 and formulated according to the description of Example 3 was confirmed.

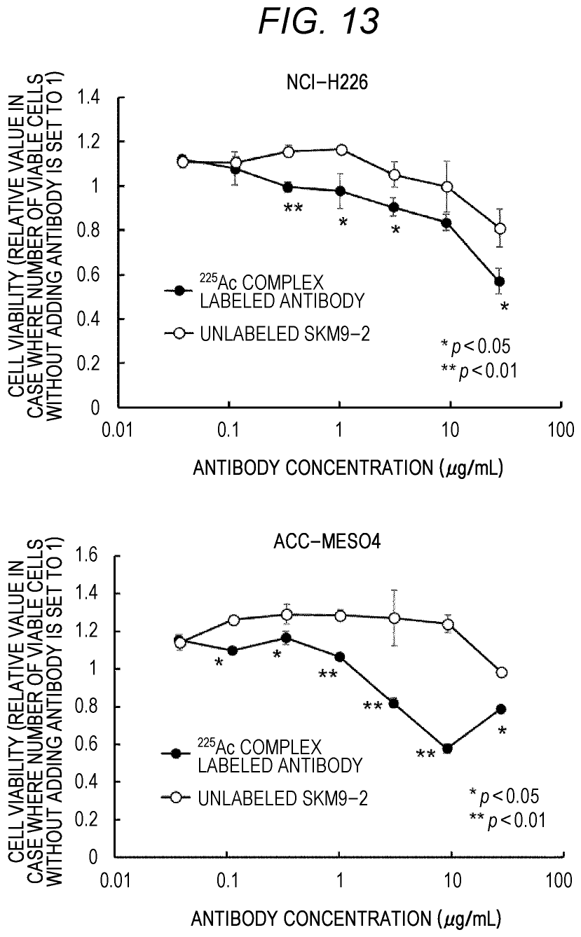

The HEG1-positive NCI-H226 cells (purchased from ATCC) and the ACC-MESO4 cells (Purchased from RIKEN BRC) were cultured in RPMI1640 medium (contains 10% fetal bovine serum (BioWest), 1% penicillin-streptomycin (Gibco)), and the ²²⁵Ac complex labeled antibodies were added to final concentration of 0, 0.04, 0.12, 0.37, 1.1, 3.3, 10.0, and 30.0 kBq/mL (0, 0.04, 0.11, 0.34, 1.01, 3.03, 9.09, and 27.3 μg/mL as SKM9-2) to the cells. In addition, unlabeled SKM9-2 was added to have the same antibody concentration as the sample of each ²²⁵Ac complex labeled antibody, and cultured. 144 hours after addition of the sample, CellTiter-Glo™ 2.0 Cell Viability Assay (manufactured by Promega Corporation) was added to the medium, chemiluminescence was detected using a microplate reader (SpectraMax i3x, manufactured by Molecular Devices, LLC.), and the number of living cells was calculated. The ratio of the calculated number of living cells to the number of living cells under the condition where the antibody was not added was taken to evaluate the cell-killing effect. The results are shown in FIG. 13. The vertical axis of the graph represents a relative value when the number of living cells under the condition where the antibody was not added is 1, and the horizontal axis represents the final concentration of the added antibody. Graph plots and error bars represent mean±standard deviation of each group (n=3). Student's t-test (two-sided test) was performed using Microsoft Excel 2016 (manufactured by Microsoft Corporation) on the living cell ratio of each cell between the ²²⁵Ac complex labeled antibody group and the unlabeled SKM9-2 group under the same condition of the added antibody amount, and a statistically significant difference test was performed.

As shown in FIG. 13, in both NCI-H226 cells and ACC-MESO4 cells, the cell-killing effect was not confirmed in the unlabeled SKM9-2, but the cell-killing effect was confirmed in the ²²⁵Ac complex labeled antibody. In the statistical comparison regarding the cell-killing effect in the group to which the ²²⁵Ac complex labeled antibody was added and the group to which the unlabeled SKM9-2 was added, under the conditions where the concentration of the added ²²⁵Ac complex labeled antibody in the NCI-H226 cells was 0.34, 1.01, 3.03, and 27.3 μg/mL, and under the condition where the concentration of the added ²²⁵Ac complex labeled antibody in the ACC-MESO4 cells was 0.11 to 27.3 μg/mL, a statistically significant difference (p<0.05 or p<0.01) was observed with respect to the unlabeled antibody control group.

### Evaluation 2: Evaluation of Stability of ⁸⁹Zr Complex Labeled Antibody

The ⁸⁹Zr complex labeled antibody obtained in Example 3 was stored at room temperature (24.5 to 25.5° C.) for 1 week, and at each time point (0 day point, 3 day point, and 7 day point), the radiochemical purity, the ratio of aggregates, and the antigen binding activity to living cells were evaluated. In addition, the antigen binding activity to tumor sections is evaluated using the radioactive complex produced in the same manner. The ⁸⁹Zr complex labeled antibody used in the following evaluation was produced according to the description of Example 2, and formulated according to the description of Example 3.

### Evaluation 2-1: Radiochemical Purity

The radiochemical purity was analyzed using TLC. The conditions for TLC were the same as those used when the reaction rate was evaluated in Example 1. The results are shown in Table 6.

Radiochemical purity (%)

| | 0 day point | 3 day point | 7 day point |
|---|---|---|---|
| ⁸⁹Zr complex | 85.0 | 87.8 | 86.3 |
| labeled antibody | | | |

In a case where the ⁸⁹Zr complex labeled antibody was stored at room temperature for 7 days after production, the radiochemical purity was maintained at 86% or more.

### Evaluation 2-2: Ratio of Aggregates

The ratio of aggregates contained in the ⁸⁹Zr complex labeled antibody was confirmed by SEC. SEC conditions were the same as those in Evaluation 1-2. Table 7 shows the ratio of each component in a case where the product is stored for 7 days after completion of production.

| | Main peak | Aggregate peak |
|---|---|---|
| | percentage (%) | percentage (%) |
| ⁸⁹Zr complex labeled | 96.52 | 1.21 |
| antibody (Produced 0 | | |
| days after) | | |
| ⁸⁹Zr complex labeled | 89.00 | 8.58 |
| antibody (Produced 3 | | |
| days after) | | |
| ⁸⁹Zr complex labeled | 62.85 | 33.04 |
| antibody (Produced 7 | | |
| days after) | | |

### Evaluation 2-3: Antigen Binding Activity on Living Cell

HEG1-positive NCI-H226 (purchased from ATCC), ACC-MESO4 (purchased from RIKEN BRC), and HEG1-negative A549 (purchased from ECACC) were suspended in RPMI1640 medium (manufactured by Gibco) (contains 10% fetal bovine serum (manufactured by BioWest), 1% penicillin-streptomycin (manufactured by Gibco)) or Ham's F-12K medium (manufactured by Gibco) (contains 10% fetal bovine serum (manufactured by ATCC), 1% penicillin-streptomycin (manufactured by Gibco)), and it was incubated in a 24-well plate (1.0×10⁵ cells/well) at 37° C. under conditions of 5% carbon dioxide and 21% oxygen for 24 hours. After removing the medium, 0.4 mL of a medium containing an ⁸⁹Zr complex labeled antibody (finally added radioactivity 2 kBq/well) was added to each well, and the plate was incubated at 37° C. for 1 hour under atmospheric conditions. Separately, in order to confirm the HEG1 specificity of the ⁸⁹Zr complex labeled antibody, incubation was performed in the same condition except that unlabeled SKM9-2 (final concentration: 0.5 μM) was added as a competing compound for the ⁸⁹Zr complex labeled antibody. Thereafter, each well was washed with 0.5 mL of PBS, and the cells were dissolved in 0.15 mL of a 2 N aqueous sodium hydroxide solution (manufactured by NACALAI TESQUE, INC.) at room temperature. Thereafter, 0.15 mL of ultrapure water (manufactured by Millipore) was added to each well to make the solution volume 0.3 mL, and 0.2 mL of the solution was fractionated, and radioactivity in the solution was measured with a gamma counter (model name: 2470 WIZARD² autogamma counter, manufactured by PerkinElmer Inc). The solution remaining in the well was dried completely and dissolved in 0.4 mL of ultrapure water, and then the absorbance at 562 nm was measured with a plate reader (SpectraMax i3x, manufactured by Molecular Devices, LLC.) using Pierce BCA Protein Assay Kit (manufactured by Thermo Fisher Scientific Inc.), and the protein concentration of each well was calculated. Among the radioactivity added to each well, a value (Uptake ratio (% addition amount/mg)) obtained by correcting the ratio of the radioactivity bound to the cell by the protein concentration in the well was calculated, and a parametric Tukey-type multiple comparison test was performed using Stat Preclinica (manufactured by Takumi Information Technology Inc.) to perform a significant difference test.

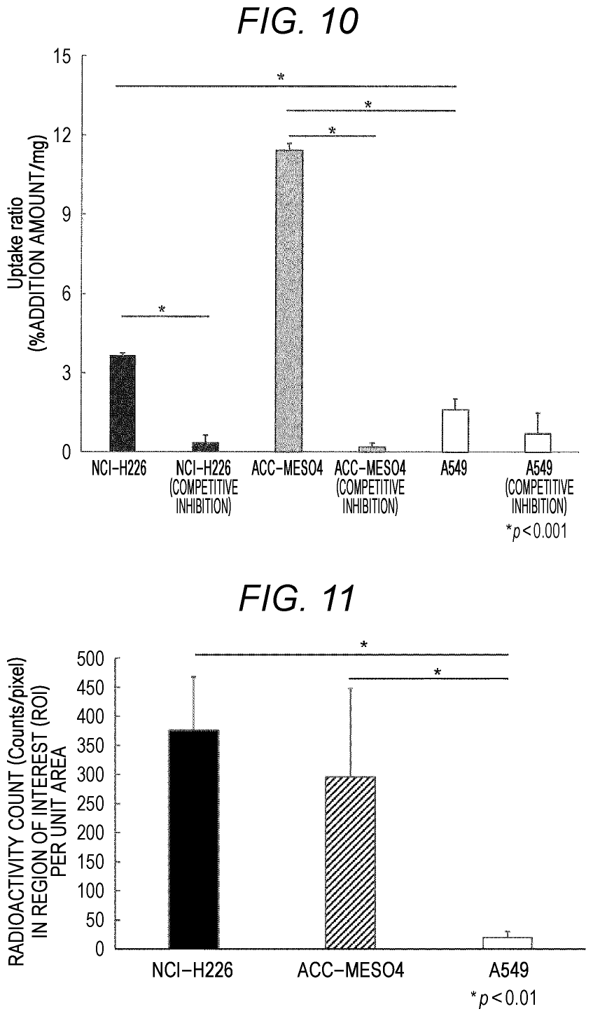

The results for the in vitro cell binding and specificity to the HEG1 of the ⁸⁹Zr complex labeled antibody are illustrated in FIG. 10. In the NCI-H226 cell group and the ACC-MESO4 cell group, a statistically significant difference was observed with respect to the A549 cell group (p<0.001). In addition, in the presence of the competing compound SKM9-2, in NCI-H226 cells and ACC-MESO4 cells, a statistically significant inhibitory effect was confirmed as compared with the absence of the competing compound (p<0.001). On the other hand, in HEG1-negative A549 cells, no competitive inhibition was observed regardless of the presence or absence of the competing compound, and no statistically significant difference at the 5% significance level was observed. From these, it was shown that the ⁸⁹Zr complex labeled antibody has binding and specificity to the HEG1.

### Evaluation 2-4: Antigen Binding Activity to Tumor Section

The antigen binding activity of the ⁸⁹Zr complex labeled antibody to tumor sections is confirmed by in vitro ARG (production date (day 0) only). Evaluation is performed according to the method described in Evaluation 1-5 except that the radioactive concentration of the solution in which the HEG1-positive tumor sections and the negative tumor section are immersed is changed to 5 kBq/mL and the radioactive complex to be added is changed to an ⁸⁹Zr complex labeled antibody. The binding of the ⁸⁹Zr complex labeled antibody is confirmed only in the HEG1-positive tumor section, and the HEG1 selective binding activity is confirmed.

### Evaluation 3: Evaluation of Tumor Accumulation of ⁸⁹Zr Complex Labeled Antibody

The HEG1-positive tumor cells were transplanted into mice to prepare a subcutaneous tumor-bearing model of HEG1-positive cells, and the tumor accumulation of the ⁸⁹Zr complex labeled antibody in the model was confirmed. The ⁸⁹Zr complex labeled antibody used in the following evaluation was produced according to the description of Example 2, and formulated according to the description of Example 3.

Specifically, HEG1 positive NCI-H226 cells (purchased from ATCC) were injected to a 5-week-old female ICR-nu/nu mouse (provided by Charles River Laboratories Japan, Inc.) subcutaneously in the flank of the mouse at 2×10⁶ cells to prepare a tumor-bearing mouse. After 4 weeks from the tumor-bearing treatment, the tumor volume was confirmed to be approximately 100 to 300 mm³, and the ⁸⁹Zr complex labeled antibody was injected through the tail vein at a dose of about 4 MBq/animal (n=3). Imaging was performed using small animal PET (Positron Emission Tomography)/CT (Computed Tomography) 24, 48, 96, and 168 hours after injection. As imaging conditions of PET, acquisition time was 600 seconds (24, 48 hours after injection), 1200 seconds (96, 168 hours after injection), and energy window was 357.7 to 664.3 keV. Image reconstruction by Maximum Likelihood-Expectation Maximization method (Iterations: 12) incorporating each correction (Scatter correction, random correction, attenuation correction) was performed to obtain a PET image. The quantitative value of the obtained PET image was converted into a ratio of the radioactive concentration (Standard Uptake Value: SUV) of each site when the radioactive concentration in a case where it was assumed that the injected radioactive substance was uniformly distributed to whole body and not excreted was 1, using image analysis software PMOD (manufactured by PMOD).

The tumor volume was calculated according to the following formula.

$$Tumor volume ( {mm}^{3} ) = ( major tumor diameter \times {( minor tumor diameter )}^{2} ) \times 1 / 2$$

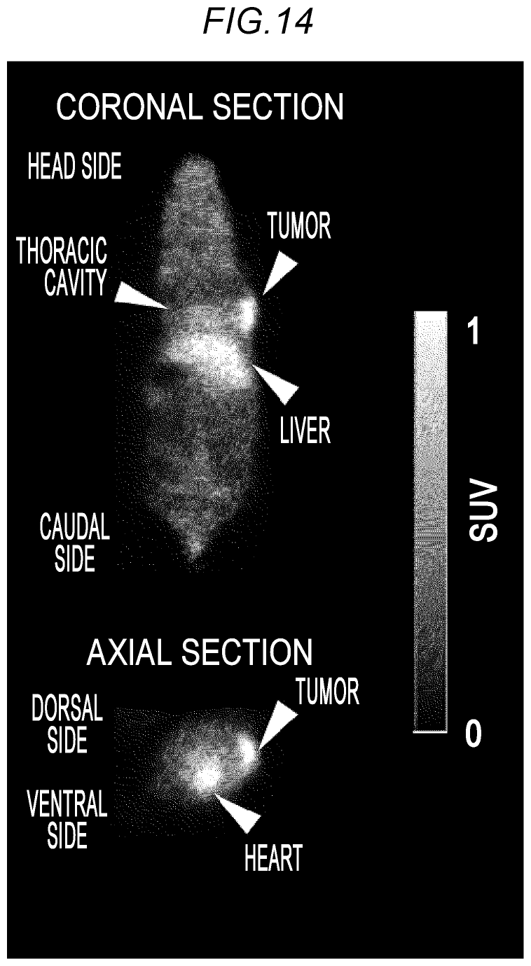

The PET imaging results 96 hours after injection are illustrated in FIG. 14. Bars with gradations in FIG. 14 reflect SUVs, with arrows indicating heart, chest cavity, tumor, and liver. As a quantitative analysis, the volume of interest was set in the tumor, the heart (reflecting the radioactive concentration in the blood), the chest cavity, and the muscle (chest dorsal side) on the SUV-converted PET image, and the maximum value (SUVₘₐₓ) of the SUV of the tumor and the average value (SUVₘₑₐₙ) of the SUVs of the heart, chest cavity, and muscle were acquired. The tumor/organ ratio was calculated according to the following formula.

Tumor/organ ratio=SUVₘₐₓ of tumor/SUVₘₑₐₙ of each organ

The results of the calculated tumor/organ ratio are illustrated in Table 8.

| | Tumor/organ ratio |
|---|---|
| | Mean ± standard deviation, n = 3 |
| Tumor/heart ratio | 1.10 ± 0.37 |
| Tumor/thoracic cavity ratio | 2.91 ± 0.41 |
| Tumor/muscle ratio | 5.19 ± 2.29 |

As a result of PET imaging, the tumor-organ ratio of the ⁸⁹Zr complex labeled antibody in the heart, chest cavity, or muscle was higher than 1, indicating that radioactivity was accumulated in the tumor at a higher concentration than in the normal organs, and the ⁸⁹Zr complex labeled antibody visualized a HEG1-positive tumor.

### Evaluation 4: Evaluation of Drug Efficacy

Using a mouse, a subcutaneous tumor-bearing model of HEG1 positive cells is prepared, and the antitumor effect of the ²²⁵Ac complex labeled antibody is confirmed. The ²²⁵Ac complex labeled antibody used in the following evaluation is produced according to the description of Example 1, and formulated according to the description of Example 3.

The HEG1-positive cell-bearing mice are prepared in the same manner as in Evaluation 3. The tumor volume is confirmed to be approximately 100 to 300 mm³, and grouping is performed randomly from individuals having a shape suitable for tumor diameter measurement. The ²²⁵Ac complex labeled antibody is injected tail-intravenously at doses of 2.5, 5.0, and 10.0 kBq/animal (50 μg/animal as antibody). In addition, as the control group, a group (antibody control group) to which an unlabeled antibody (IgG4PE) having the same antibody amount as the ²²⁵Ac complex labeled antibody is injected and a vehicle group to which a preservation buffer is injected are set. There are 6 animals in each group, and general condition observation, body weight measurement, and tumor volume measurement are performed over time until 60 days after injection.

In the group to which the ²²⁵Ac complex labeled antibody was injected, a significant difference in antitumor effect was observed at the final observation day as compared with the two control groups (Antibody control group, Vehicle group). In addition, there is no significant differences in general condition in each group, and no side effects such as significant weight loss are observed.

### Example 4: Production of Peptide-Modified Antibody Using Antibody (IgG1) of Present Disclosure

A peptide-modified antibody is produced according to “1-2. Modification of SKM9-2 with antibody modification linker s” in Example 1 except that the antibody (IgG1) of the present disclosure produced according to Production Example 1 is used. In the resulting peptide-modified antibody, the Fc region of the antibody is site-specifically modified with the peptide described above.

### Example 5: Production of Peptide-Modified Antibody Using Antibody (IgG2) of Present Disclosure

A peptide-modified antibody is produced according to “1-2. Modification of SKM9-2 with antibody modification linkers” in Example 1 except that the antibody (IgG2) of the present disclosure produced according to Production Example 1 is used. In the resulting peptide-modified antibody, the Fc region of the antibody is site-specifically modified with the peptide described above.

### Example 6: Production of Peptide-Modified Antibody Using Antibody (IgG4) of Present Disclosure

A peptide-modified antibody is produced according to “1-2. Modification of SKM9-2 with antibody modification linkers” in Example 1 except that the antibody of the present disclosure (IgG4) produced according to Production Example 1 is used. In the resulting peptide-modified antibody, the Fc region of the antibody is site-specifically modified with the peptide described above.

Correspondence table between sequence numbers and their contents in sequence list

| | |
|---|---|
| SEQ ID NO: 1 | xiH |
| SEQ ID NO: 2 | zuH3 |
| SEQ ID NO: 3 | zuH5 |
| SEQ ID NO: 4 | zuH3a |
| SEQ ID NO: 5 | zuH3b |
| SEQ ID NO: 6 | zuH3c |
| SEQ ID NO: 7 | zuH3d |
| SEQ ID NO: 8 | zuH3e |
| SEQ ID NO: 9 | zuH3f |
| SEQ ID NO: 10 | xiL |
| SEQ ID NO: 11 | zuL2 |
| SEQ ID NO: 12 | zuL3 |
| SEQ ID NO: 13 | zuL4 |
| SEQ ID NO: 14 | zuL5 |
| SEQ ID NO: 15 | zuL5a |
| SEQ ID NO: 16 | zuL5b |
| SEQ ID NO: 17 | zuL5c |
| SEQ ID NO: 18 | zuL5d |
| SEQ ID NO: 19 | zuL5e |
| SEQ ID NO: 20 | zuL5f |
| SEQ ID NO: 21 | zuL5g |
| SEQ ID NO: 22 | zuL5h |
| SEQ ID NO: 23 | zuL5i |
| SEQ ID NO: 24 | zuL5j |
| SEQ ID NO: 25 | zuL5k |
| SEQ ID NO: 26 | zuL5L |
| SEQ ID NO: 27 | zuL5m |
| SEQ ID NO: 28 | zuL5n |
| SEQ ID NO: 29 | zuL5o |
| SEQ ID NO: 30 | zuL5p |
| SEQ ID NO: 31 | zuL5q |
| SEQ ID NO: 32 | H-FR1 |
| SEQ ID NO: 33 | |
| SEQ ID NO: 34 | |
| SEQ ID NO: 35 | |
| SEQ ID NO: 36 | |
| SEQ ID NO: 37 | H-CDR1 |
| SEQ ID NO: 38 | |
| SEQ ID NO: 39 | |
| SEQ ID NO: 40 | H-FR2 |
| SEQ ID NO: 41 | |
| SEQ ID NO: 42 | |
| SEQ ID NO: 43 | H-CDR2 |
| SEQ ID NO: 44 | |
| SEQ ID NO: 45 | |
| SEQ ID NO: 46 | H-FR3 |
| SEQ ID NO: 47 | |
| SEQ ID NO: 48 | |
| SEQ ID NO: 49 | |
| SEQ ID NO: 50 | |
| SEQ ID NO: 51 | H-CDR3 |
| SEQ ID NO: 52 | |
| SEQ ID NO: 53 | |
| SEQ ID NO: 54 | H-FR4 |
| SEQ ID NO: 55 | |
| SEQ ID NO: 56 | |
| SEQ ID NO: 57 | L-FR1 |
| SEQ ID NO: 58 | |
| SEQ ID NO: 59 | |
| SEQ ID NO: 60 | |
| SEQ ID NO: 61 | |
| SEQ ID NO: 62 | L-CDR1 |
| SEQ ID NO: 63 | |
| SEQ ID NO: 64 | |
| SEQ ID NO: 65 | |
| SEQ ID NO: 66 | |
| SEQ ID NO: 67 | |
| SEQ ID NO: 68 | |
| SEQ ID NO: 69 | |
| SEQ ID NO: 70 | |
| SEQ ID NO: 71 | |
| SEQ ID NO: 72 | L-FR2 |
| SEQ ID NO: 73 | |
| SEQ ID NO: 74 | |
| SEQ ID NO: 75 | L-CDR2 |
| SEQ ID NO: 76 | |
| SEQ ID NO: 77 | |
| SEQ ID NO: 78 | |
| SEQ ID NO: 79 | L-FR3 |
| SEQ ID NO: 80 | |
| SEQ ID NO: 81 | |
| SEQ ID NO: 82 | L-CDR3 |
| SEQ ID NO: 83 | |
| SEQ ID NO: 84 | |
| SEQ ID NO: 85 | |
| SEQ ID NO: 86 | L-FR4 |
| SEQ ID NO: 87 | |
| SEQ ID NO: 88 | |
| SEQ ID NO: 89 | xiH-CDR1 |
| SEQ ID NO: 90 | xiH-CDR2 |
| SEQ ID NO: 91 | xiH-CDR3 |
| SEQ ID NO: 92 | zuH3-CDR1 |
| SEQ ID NO: 93 | zuH3-CDR2 |
| SEQ ID NO: 94 | zuH3-CDR3 |
| SEQ ID NO: 95 | zuH5-CDR1 |
| SEQ ID NO: 96 | zuH5-CDR2 |
| SEQ ID NO: 97 | zuH5-CDR3 |
| SEQ ID NO: 98 | zuH3a-CDR1 |
| SEQ ID NO: 99 | zuH3a-CDR2 |
| SEQ ID NO: 100 | zuH3a-CDR3 |
| SEQ ID NO: 101 | zuH3b-CDR1 |
| SEQ ID NO: 102 | zuH3b-CDR2 |
| SEQ ID NO: 103 | zuH3b-CDR3 |
| SEQ ID NO: 104 | zuH3c-CDR1 |
| SEQ ID NO: 105 | zuH3c-CDR2 |
| SEQ ID NO: 106 | zuH3c-CDR3 |
| SEQ ID NO: 107 | zuH3d-CDR1 |
| SEQ ID NO: 108 | zuH3d-CDR2 |
| SEQ ID NO: 109 | zuH3d-CDR3 |
| SEQ ID NO: 110 | zuH3e-CDR1 |
| SEQ ID NO: 111 | zuH3e-CDR2 |
| SEQ ID NO: 112 | zuH3e-CDR3 |
| SEQ ID NO: 113 | zuH3f-CDR1 |
| SEQ ID NO: 114 | zuH3f-CDR2 |
| SEQ ID NO: 115 | zuH3f-CDR3 |
| SEQ ID NO: 116 | xiL-CDR1 |
| SEQ ID NO: 117 | xiL-CDR2 |
| SEQ ID NO: 118 | xiL-CDR3 |
| SEQ ID NO: 119 | zuL2-CDR1 |
| SEQ ID NO: 120 | zuL2-CDR2 |
| SEQ ID NO: 121 | zuL2-CDR3 |
| SEQ ID NO: 122 | zuL3-CDR1 |
| SEQ ID NO: 123 | zuL3-CDR2 |
| SEQ ID NO: 124 | zuL3-CDR3 |
| SEQ ID NO: 125 | zuL4-CDR1 |
| SEQ ID NO: 126 | zuL4-CDR2 |
| SEQ ID NO: 127 | zuL4-CDR3 |
| SEQ ID NO: 128 | zuL5-CDR1 |
| SEQ ID NO: 129 | zuL5-CDR2 |
| SEQ ID NO: 130 | zuL5-CDR3 |
| SEQ ID NO: 131 | zuL5a-CDR1 |
| SEQ ID NO: 132 | zuL5a-CDR2 |
| SEQ ID NO: 133 | zuL5a-CDR3 |
| SEQ ID NO: 134 | zuL5b-CDR1 |
| SEQ ID NO: 135 | zuL5b-CDR2 |
| SEQ ID NO: 136 | zuL5b-CDR3 |
| SEQ ID NO: 137 | zuL5c-CDR1 |
| SEQ ID NO: 138 | zuL5c-CDR2 |
| SEQ ID NO: 139 | zuL5c-CDR3 |
| SEQ ID NO: 140 | zuL5d-CDR1 |
| SEQ ID NO: 141 | zuL5d-CDR2 |
| SEQ ID NO: 142 | zuL5d-CDR3 |
| SEQ ID NO: 143 | zuL5e-CDR1 |
| SEQ ID NO: 144 | zuL5e-CDR2 |
| SEQ ID NO: 145 | zuL5e-CDR3 |
| SEQ ID NO: 146 | zuL5f-CDR1 |
| SEQ ID NO: 147 | zuL5f-CDR2 |
| SEQ ID NO: 148 | zuL5f-CDR3 |
| SEQ ID NO: 149 | zuL5g-CDR1 |
| SEQ ID NO: 150 | zuL5g-CDR2 |
| SEQ ID NO: 151 | zuL5g-CDR3 |
| SEQ ID NO: 152 | zuL5h-CDR1 |
| SEQ ID NO: 153 | zuL5h-CDR2 |
| SEQ ID NO: 154 | zuL5h-CDR3 |
| SEQ ID NO: 155 | zuL5i-CDR1 |
| SEQ ID NO: 156 | zuL5i-CDR2 |
| SEQ ID NO: 157 | zuL5i-CDR3 |
| SEQ ID NO: 158 | zuL5j-CDR1 |
| SEQ ID NO: 159 | zuL5j-CDR2 |
| SEQ ID NO: 160 | zuL5j-CDR3 |
| SEQ ID NO: 161 | zuL5k-CDR1 |
| SEQ ID NO: 162 | zuL5k-CDR2 |
| SEQ ID NO: 163 | zuL5k-CDR3 |
| SEQ ID NO: 164 | zuL5L-CDR1 |
| SEQ ID NO: 165 | zuL5L-CDR2 |
| SEQ ID NO: 166 | zuL5L-CDR3 |
| SEQ ID NO: 167 | zuL5m-CDR1 |
| SEQ ID NO: 168 | zuL5m-CDR2 |
| SEQ ID NO: 169 | zuL5m-CDR3 |
| SEQ ID NO: 170 | zuL5n-CDR1 |
| SEQ ID NO: 171 | zuL5n-CDR2 |
| SEQ ID NO: 172 | zuL5n-CDR3 |
| SEQ ID NO: 173 | zuL5o-CDR1 |
| SEQ ID NO: 174 | zuL5o-CDR2 |
| SEQ ID NO: 175 | zuL5o-CDR3 |
| SEQ ID NO: 176 | zuL5p-CDR1 |
| SEQ ID NO: 177 | zuL5p-CDR2 |
| SEQ ID NO: 178 | zuL5p-CDR3 |
| SEQ ID NO: 179 | zuL5q-CDR1 |
| SEQ ID NO: 180 | zuL5q-CDR2 |
| SEQ ID NO: 181 | zuL5q-CDR3 |
| SEQ ID NO: 182 | HEGl partial peptide |
| SEQ ID NO: 183 | SLURPgpi |
| SEQ ID NO: 184 | Signal sequence |
| SEQ ID NO: 185 | IgG binding peptide |
| SEQ ID NO: 186 | |
| SEQ ID NO: 187 | |
| SEQ ID NO: 188 | |
| SEQ ID NO: 189 | |
| SEQ ID NO: 190 | |
| SEQ ID NO: 191 | |
| SEQ ID NO: 192 | |
| SEQ ID NO: 193 | |
| SEQ ID NO: 194 | |
| SEQ ID NO: 195 | |
| SEQ ID NO: 196 | |
| SEQ ID NO: 197 | |
| SEQ ID NO: 198 | |
| SEQ ID NO: 199 | |
| SEQ ID NO: 200 | |
| SEQ ID NO: 201 | |
| SEQ ID NO: 202 | |
| SEQ ID NO: 203 | |
| SEQ ID NO: 204 | |
| SEQ ID NO: 205 | |
| SEQ ID NO: 206 | |
| SEQ ID NO: 207 | |
| SEQ ID NO: 208 | |
| SEQ ID NO: 209 | |
| SEQ ID NO: 210 | |
| SEQ ID NO: 211 | |
| SEQ ID NO: 212 | |
| SEQ ID NO: 213 | |
| SEQ ID NO: 214 | |
| SEQ ID NO: 215 | IgG1 CH |
| SEQ ID NO: 216 | IgG1 hinge region |
| SEQ ID NO: 217 | IgG4 hinge region |
| SEQ ID NO: 218 | IgG4PE hinge region |
| SEQ ID NO: 219 | IgG1 CH2 region |
| SEQ ID NO: 220 | IgG4 or IgG4PE CH2 region |
| SEQ ID NO: 221 | Heavy chain signal sequence VH1 |
| SEQ ID NO: 222 | Heavy chain signal sequence VH2 |
| SEQ ID NO: 223 | Heavy chain signal sequence VH3 |
| SEQ ID NO: 224 | Heavy chain signal sequence VH4 |
| SEQ ID NO: 225 | Heavy chain signal sequence VH5 |
| SEQ ID NO: 226 | Heavy chain signal sequence VH6 |
| SEQ ID NO: 227 | Light chain signal sequence KL1 |
| SEQ ID NO: 228 | Light chain signal sequence KL2 |
| SEQ ID NO: 229 | Mouse heavy chain signal sequence |
| SEQ ID NO: 230 | Mouse light chain signal sequence |

1. A complex comprising:
an antibody or antigen-binding fragment thereof; and
a radionuclide, wherein
the antibody is a humanized antibody capable of binding a human HEG1 protein expressed on mesothelioma cells.

2. The complex according to claim 1, wherein
the antibody includes a heavy chain variable region including a heavy chain CDR1 having an amino acid sequence set forth in SEQ ID NO: 37, a heavy chain CDR2 having an amino acid sequence set forth in SEQ ID NO: 43, and a heavy chain CDR3 having an amino acid sequence set forth in SEQ ID NO: 51, and a light chain variable region including a light chain CDR1 having an amino acid sequence set forth in SEQ ID NO: 62, a light chain CDR2 having an amino acid sequence set forth in SEQ ID NO: 75, and a light chain CDR3 having an amino acid sequence set forth in SEQ ID NO: 82.

3. The complex according to claim 1, wherein
the antibody includes a heavy chain variable region including a heavy chain CDR1 having an amino acid sequence set forth in SEQ ID NO: 37, a heavy chain CDR2 having an amino acid sequence set forth in SEQ ID NO: 43, and a heavy chain CDR3 having an amino acid sequence set forth in SEQ ID NO: 51, and a light chain variable region including a light chain CDR1 having an amino acid sequence set forth in SEQ ID NO: 63, a light chain CDR2 having an amino acid sequence set forth in SEQ ID NO: 75, and a light chain CDR3 having an amino acid sequence set forth in SEQ ID NO: 82.

4. The complex according to claim 1, wherein
the heavy chain variable region includes
(1) a heavy chain CDR1 having an amino acid sequence set forth in SEQ ID NO: 89, a heavy chain CDR2 having an amino acid sequence set forth in SEQ ID NO: 90, and a heavy chain CDR3 having an amino acid sequence set forth in SEQ ID NO: 91;
(2) a heavy chain CDR1 having an amino acid sequence set forth in SEQ ID NO: 92, a heavy chain CDR2 having an amino acid sequence set forth in SEQ ID NO: 93, and a heavy chain CDR3 having an amino acid sequence set forth in SEQ ID NO: 94;
(3) a heavy chain CDR1 having an amino acid sequence set forth in SEQ ID NO: 95, a heavy chain CDR2 having an amino acid sequence set forth in SEQ ID NO: 96, and a heavy chain CDR3 having an amino acid sequence set forth in SEQ ID NO: 97;
(4) a heavy chain CDR1 having an amino acid sequence set forth in SEQ ID NO: 98, a heavy chain CDR2 having an amino acid sequence set forth in SEQ ID NO: 99, and a heavy chain CDR3 having an amino acid sequence set forth in SEQ ID NO: 100;
(5) a heavy chain CDR1 having an amino acid sequence set forth in SEQ ID NO: 101, a heavy chain CDR2 having an amino acid sequence set forth in SEQ ID NO: 102, and a heavy chain CDR3 having an amino acid sequence set forth in SEQ ID NO: 103;
(6) a heavy chain CDR1 having an amino acid sequence set forth in SEQ ID NO: 104, a heavy chain CDR2 having an amino acid sequence set forth in SEQ ID NO: 105, and a heavy chain CDR3 having an amino acid sequence set forth in SEQ ID NO: 106;
(7) a heavy chain CDR1 having an amino acid sequence set forth in SEQ ID NO: 110, a heavy chain CDR2 having an amino acid sequence set forth in SEQ ID NO: 111, and a heavy chain CDR3 having an amino acid sequence set forth in SEQ ID NO: 112; or
(8) a heavy chain CDR1 having an amino acid sequence set forth in SEQ ID NO: 113, a heavy chain CDR2 having an amino acid sequence set forth in SEQ ID NO: 114, and a heavy chain CDR3 having an amino acid sequence set forth in SEQ ID NO: 115.

5. The complex according to claim 1, wherein
the light chain variable region includes:
(1) a light chain CDR1 having an amino acid sequence set forth in SEQ ID NO: 116, a light chain CDR2 having an amino acid sequence set forth in SEQ ID NO: 117, and a light chain CDR3 having an amino acid sequence set forth in SEQ ID NO: 118;
(2) a light chain CDR1 having an amino acid sequence set forth in SEQ ID NO: 119, a light chain CDR2 having an amino acid sequence set forth in SEQ ID NO: 120, and a light chain CDR3 having an amino acid sequence set forth in SEQ ID NO: 121;
(3) a light chain CDR1 having an amino acid sequence set forth in SEQ ID NO: 122, a light chain CDR2 having an amino acid sequence set forth in SEQ ID NO: 123, and a light chain CDR3 having an amino acid sequence set forth in SEQ ID NO: 124;
(4) a light chain CDR1 having an amino acid sequence set forth in SEQ ID NO: 128, a light chain CDR2 having an amino acid sequence set forth in SEQ ID NO: 129, and a light chain CDR3 having an amino acid sequence set forth in SEQ ID NO: 130;
(5) a light chain CDR1 having an amino acid sequence set forth in SEQ ID NO: 131, a light chain CDR2 having an amino acid sequence set forth in SEQ ID NO: 132, and a light chain CDR3 having an amino acid sequence set forth in SEQ ID NO: 133;
(6) a light chain CDR1 having an amino acid sequence set forth in SEQ ID NO: 134, a light chain CDR2 having an amino acid sequence set forth in SEQ ID NO: 135, and a light chain CDR3 having an amino acid sequence set forth in SEQ ID NO: 136;
(7) a light chain CDR1 having an amino acid sequence set forth in SEQ ID NO: 137, a light chain CDR2 having an amino acid sequence set forth in SEQ ID NO: 138, and a light chain CDR3 having an amino acid sequence set forth in SEQ ID NO: 139;
(8) a light chain CDR1 having an amino acid sequence set forth in SEQ ID NO: 140, a light chain CDR2 having an amino acid sequence set forth in SEQ ID NO: 141, and a light chain CDR3 having an amino acid sequence set forth in SEQ ID NO: 142;
(9) a light chain CDR1 having an amino acid sequence set forth in SEQ ID NO: 143, a light chain CDR2 having an amino acid sequence set forth in SEQ ID NO: 144, and a light chain CDR3 having an amino acid sequence set forth in SEQ ID NO: 145;
(10) a light chain CDR1 having an amino acid sequence set forth in SEQ ID NO: 146, a light chain CDR2 having an amino acid sequence set forth in SEQ ID NO: 147, and a light chain CDR3 having an amino acid sequence set forth in SEQ ID NO: 148;
(11) a light chain CDR1 having an amino acid sequence set forth in SEQ ID NO: 149, a light chain CDR2 having an amino acid sequence set forth in SEQ ID NO: 150, and a light chain CDR3 having an amino acid sequence set forth in SEQ ID NO: 151;
(12) a light chain CDR1 having an amino acid sequence set forth in SEQ ID NO: 152, a light chain CDR2 having an amino acid sequence set forth in SEQ ID NO: 153, and a light chain CDR3 having an amino acid sequence set forth in SEQ ID NO: 154;
(13) a light chain CDR1 having an amino acid sequence set forth in SEQ ID NO: 155, a light chain CDR2 having an amino acid sequence set forth in SEQ ID NO: 156, and a light chain CDR3 having an amino acid sequence set forth in SEQ ID NO: 157;
(14) a light chain CDR1 having an amino acid sequence set forth in SEQ ID NO: 158, a light chain CDR2 having an amino acid sequence set forth in SEQ ID NO: 159, and a light chain CDR3 having an amino acid sequence set forth in SEQ ID NO: 160;
(15) a light chain CDR1 having an amino acid sequence set forth in SEQ ID NO: 161, a light chain CDR2 having an amino acid sequence set forth in SEQ ID NO: 162, and a light chain CDR3 having an amino acid sequence set forth in SEQ ID NO: 163;
(16) a light chain CDR1 having an amino acid sequence set forth in SEQ ID NO: 173, a light chain CDR2 having an amino acid sequence set forth in SEQ ID NO: 174, and a light chain CDR3 having an amino acid sequence set forth in SEQ ID NO: 175;
(17) a light chain CDR1 having an amino acid sequence set forth in SEQ ID NO: 176, a light chain CDR2 having the amino acid sequence set forth in SEQ ID NO: 177, and a light chain CDR3 having an amino acid sequence set forth in SEQ ID NO: 178; or
(18) a light chain CDR1 having an amino acid sequence set forth in SEQ ID NO: 179, a light chain CDR2 having an amino acid sequence set forth in SEQ ID NO: 180, and a light chain CDR3 having an amino acid sequence set forth in SEQ ID NO: 181.

6. The complex according to claim 1, further comprising:
any of the heavy chain variable regions defined in claim 4; and
any of the light chain variable regions defined in claim 5.

7. The complex according to claim 2, wherein
the heavy chain variable region includes a heavy chain CDR1 having an amino acid sequence set forth in SEQ ID NO: 104, a heavy chain CDR2 having an amino acid sequence set forth in SEQ ID NO: 105, and a heavy chain CDR3 having an amino acid sequence set forth in SEQ ID NO: 106, and
the light chain variable region includes a light chain CDR1 having an amino acid sequence set forth in SEQ ID NO: 149, a light chain CDR2 having an amino acid sequence set forth in SEQ ID NO: 150, and a light chain CDR3 having an amino acid sequence set forth in SEQ ID NO: 151.

8. The complex according to claim 2, wherein the heavy chain variable region includes a framework region 1 having an amino acid sequence set forth in SEQ ID NO: 32, a framework region 2 having an amino acid sequence set forth in SEQ ID NO: 40, a framework region 3 having an amino acid sequence set forth in SEQ ID NO: 46, and a framework region 4 having an amino acid sequence set forth in SEQ ID NO: 54.

9. The complex according to claim 2, wherein the light chain variable region includes a framework region 1 having an amino acid sequence set forth in SEQ ID NO: 57, a framework region 2 having an amino acid sequence set forth in SEQ ID NO: 72, a framework region 3 having an amino acid sequence set forth in SEQ ID NO: 79, and a framework region 4 having an amino acid sequence set forth in SEQ ID NO: 86.

10. The complex according to claim 1, wherein
the heavy chain variable region has an amino acid sequence selected from the group consisting of SEQ ID NOs: 1 to 6, 8, and 9, and
the light chain variable region has an amino acid sequence selected from the group consisting of SEQ ID NOs: 10, 12 to 15, 17 to 21, and 29 to 31.

11. The complex according to claim 1, wherein
the heavy chain variable region has an amino acid sequence set forth in SEQ ID NO: 6, and
the light chain variable region has an amino acid sequence set forth in SEQ ID NO: 21.

12. The complex according to claim 1, wherein
the radionuclide is a metal radionuclide,
the antibody and the metal radionuclide are linked via a linker,
the linker has a chelate site, and
the metal radionuclide is chelated to the chelate site.

13. The complex according to claim 12, wherein
the linker has a peptide having an amino acid sequence consisting of 13 to 17 amino acid residues represented by Formula I below:
(X₁₋₃)-C-(X₂)—H-(Xaa1)-G-(Xaa2)-L-V-W-C-(X₁₋₃) (I)
{wherein, each X is independently any amino acid residue other than cysteine,
Xaa1 is a lysine residue, a cysteine residue, an aspartic acid residue, a glutamic acid residue, 2-aminosuberic acid, or diaminopropionic acid, and
Xaa2 is a glutamic acid residue, a glutamine residue, or an asparagine residue}, and a chelate site that chelates the radionuclide linked to the peptide, and
the peptide is cross-linked with the antibody.

14. The complex according to claim 13, wherein the number of bonds of the linker to the antibody is 1.

15. The complex according to claim 1, wherein the radionuclide is a diagnostic metal radionuclide.

16. The complex according to claim 1, wherein the radionuclide is a therapeutic metal radionuclide.

17. A composition comprising the complex according to claim 1.

18. A composition comprising:
the complex according to claim 1; and
a pharmaceutically acceptable carrier, excipient or additive.


SEQUENCE LISTING (230 sequences)

SEQ ID NO: 1 - protein, 113 aa, Artificial Sequence
  xiH
QVQLQQPGAELVRPGASVKLSCKASGFSFTTYWITWVKQRPGQGLEWIGMIHPSDSETRLSQTFKDKATLTVDKSSNTAYMQFSSPTSEDSAVYYCVRDFDVWGAGTTVTVSS

SEQ ID NO: 2 - protein, 113 aa, Artificial Sequence
  zuH3
QVQLVQSGAEVKKPGASVKLSCKASGFSFTTYWITWVRQAPGQGLEWIGMIHPSDSETRLSQTFKDKATLTADKSTNTAYMELSSLRSEDTAVYYCARDFDVWGQGTLVTVSS

SEQ ID NO: 3 - protein, 113 aa, Artificial Sequence
  zuH5
QVQLVQSGAEVKKPGASVKLSCKASGFSFTTYWITWVRQAPGQGLEWIGMIHPSDSETRLSQTFKDKATLTADESTNTAYMELSSLRSEDTAVYYCARDFDVWGQGTLVTVSS

SEQ ID NO: 4 - protein, 113 aa, Artificial Sequence
  zuH3a
QVQLVQSGAEVKKPGASVKLSCKASGFSFTTYWITWVRQAPGQGLEWIGMIHPSDAETRLSQTFKDKATLTADKSTNTAYMELSSLRSEDTAVYYCARDFDVWGQGTLVTVSS

SEQ ID NO: 5 - protein, 113 aa, Artificial Sequence
  zuH3b
QVQLVQSGAEVKKPGASVKVSCKASGFSFTTYWITWVRQAPGQGLEWIGMIHPSDAETRLSQTFKDKATLTADKSTNTAYMELSSLRSEDTAVYYCARDFDVWGQGTLVTVSS

SEQ ID NO: 6 - protein, 113 aa, Artificial Sequence
  zuH3c
QVQLVQSGAEVKKPGASVKVSCKASGFSFTTYWITWVRQAPGQGLEWIGMIHPSDAETRLSQTFKDKATLTADKSTSTAYMELSSLRSEDTAVYYCARDFDVWGQGTLVTVSS

SEQ ID NO: 7 - protein, 113 aa, Artificial Sequence
  zuH3d
QVQLVQSGAEVKKPGASVKVSCKASGFSFTTYWITWVRQAPGQGLEWIGMIHPSDAETRLSQTFKDKATLTADKSTNTAYMELSSLRSEDTAVYYCARDFIVWGQGTLVTVSS

SEQ ID NO: 8 - protein, 113 aa, Artificial Sequence
  zuH3e
QVQLVQSGAEVKKPGASVKVSCKASGFHFTTYWITWVRQAPGQGLEWIGMIHPSDAETRLSQTFKDKATLTADKSTNTAYMELSSLRSEDTAVYYCARDFDVWGQGTLVTVSS

SEQ ID NO: 9 - protein, 113 aa, Artificial Sequence
  zuH3f
QVQLVQSGAEVKKPGASVKVSCKASGFSFRTYWITWVRQAPGQGLEWIGMIHPSDAETRLSQTFKDKATLTADKSTNTAYMELSSLRSEDTAVYYCARDFDVWGQGTLVTVSS

SEQ ID NO: 10 - protein, 112 aa, Artificial Sequence
  xiL
DVLMTQTPLSLPVSLGDQASISCRSSQNIVHNNGNTYLGWYLQKPGQSPKLLIFKVSNRFSGVPDRFSGSGSGTDFTLKISRVEAEDLGVYYCFQGSHIPRTFGGGTRLEIK

SEQ ID NO: 11 - protein, 112 aa, Artificial Sequence
  zuL2
DIQMTQSPSSLSASVGDRVTITCRSSQNIVHNNGNTYLGWYQQKPGKAPKLLIFKVSNRFSGVPSRFSGSGSGTDFTLTISSLQPEDFATYYCFQGSHIPRTFGQGTKVEIK

SEQ ID NO: 12 - protein, 112 aa, Artificial Sequence
  zuL3
DVVMTQTPLSLPVTLGQPASISCRSSQNIVHNNGNTYLGWYLQKPGQSPQLLIFKVSNRFSGVPDRFSGSGSGTDFTLKISRVEAEDVGVYYCFQGSHIPRTFGQGTRLEIK

SEQ ID NO: 13 - protein, 112 aa, Artificial Sequence
  zuL4
DVVMTQTPLSLPVTPGEPASISCRSSQNIVHNNGNTYLGWYLQKPGQSPQLLIFKVSNRFSGVPDRFSGSGSGTDFTLKISRVEAEDVGVYYCFQGSHIPRTFGQGTRLEIK

SEQ ID NO: 14 - protein, 112 aa, Artificial Sequence
  zuL5
DVVMTQSPLSLPVTLGQPASISCRSSQNIVHNNGNTYLGWYLQKPGQSPQLLIFKVSNRFSGVPDRFSGSGSGTDFTLKISRVEAEDVGVYYCFQGSHIPRTFGQGTRLEIK

SEQ ID NO: 15 - protein, 112 aa, Artificial Sequence
  zuL5a
DVVMTQSPLSLPVTLGQPASISCRSSQNIVHQNGNTYLGWYLQKPGQSPQLLIFKVSNRFSGVPDRFSGSGSGTDFTLKISRVEAEDVGVYYCFQGSHIPRTFGQGTRLEIK

SEQ ID NO: 16 - protein, 112 aa, Artificial Sequence
  zuL5b
DVVMTQSPLSLPVTLGQPASISCRSSQNIVHNQGNTYLGWYLQKPGQSPQLLIFKVSNRFSGVPDRFSGSGSGTDFTLKISRVEAEDVGVYYCFQGSHIPRTFGQGTRLEIK

SEQ ID NO: 17 - protein, 112 aa, Artificial Sequence
  zuL5c
DVVMTQSPLSLPVTLGQPASISCRSSQNIVHNNGQTYLGWYLQKPGQSPQLLIFKVSNRFSGVPDRFSGSGSGTDFTLKISRVEAEDVGVYYCFQGSHIPRTFGQGTRLEIK

SEQ ID NO: 18 - protein, 112 aa, Artificial Sequence
  zuL5d
DVVMTQSPLSLPVTLGQPASISCRSSQNIVHNNGNTYLGWYLQKPGQSPQLLIFRVSNRFSGVPDRFSGSGSGTDFTLKISRVEAEDVGVYYCFQGSHIPRTFGQGTRLEIK

SEQ ID NO: 19 - protein, 112 aa, Artificial Sequence
  zuL5e
DVVMTQSPLSLPVTLGQPASISCRSSQNIVHQNAQTYLGWYLQKPGQSPQLLIFKVSNRFSGVPDRFSGSGSGTDFTLKISRVEAEDVGVYYCFQGSHIPRTFGQGTRLEIK

SEQ ID NO: 20 - protein, 112 aa, Artificial Sequence
  zuL5f
DVVMTQSPLSLPVTLGQPASISCRSSQNIVHQNAQTYLGWYLQKPGQSPQLLIFRVSNRFSGVPDRFSGSGSGTDFTLKISRVEAEDVGVYYCFQGSHIPRTFGQGTRLEIK

SEQ ID NO: 21 - protein, 112 aa, Artificial Sequence
  zuL5g
DVVMTQSPLSLPVTLGQPASISCRSSQNIVHQNGQTYLGWYLQKPGQSPQLLIFRVSNRFSGVPDRFSGSGSGTDFTLKISRVEAEDVGVYYCFQGSHIPRTFGQGTRLEIK

SEQ ID NO: 22 - protein, 112 aa, Artificial Sequence
  zuL5h
DVVMTQSPLSLPVTLGQPASISCRSSQNIVHQHGQTYLGWYLQKPGQSPQLLIFRVSNRFSGVPDRFSGSGSGTDFTLKISRVEAEDVGVYYCFQGSHIPRTFGQGTRLEIK

SEQ ID NO: 23 - protein, 112 aa, Artificial Sequence
  zuL5i
DVVMTQSPLSLPVTLGQPASISCRSSQNIVHQTGQTYLGWYLQKPGQSPQLLIFRVSNRFSGVPDRFSGSGSGTDFTLKISRVEAEDVGVYYCFQGSHIPRTFGQGTRLEIK

SEQ ID NO: 24 - protein, 112 aa, Artificial Sequence
  zuL5j
DVVMTQSPLSLPVTLGQPASISCRSSQNIVHQNRQTYLGWYLQKPGQSPQLLIFRVSNRFSGVPDRFSGSGSGTDFTLKISRVEAEDVGVYYCFQGSHIPRTFGQGTRLEIK

SEQ ID NO: 25 - protein, 112 aa, Artificial Sequence
  zuL5k
DVVMTQSPLSLPVTLGQPASISCRSSQNIVHQNAGTYLGWYLQKPGQSPQLLIFRVSNRFSGVPDRFSGSGSGTDFTLKISRVEAEDVGVYYCFQGSHIPRTFGQGTRLEIK

SEQ ID NO: 26 - protein, 111 aa, Artificial Sequence
  zuL5L
DVVMTQSPLSLPVTLGQPASISCRSSQNIVHQNQTYLGWYLQKPGQSPQLLIFRVSNRFSGVPDRFSGSGSGTDFTLKISRVEAEDVGVYYCFQGSHIPRTFGQGTRLEIK

SEQ ID NO: 27 - protein, 111 aa, Artificial Sequence
  zuL5m
DVVMTQSPLSLPVTLGQPASISCRSSQNIVHQGQTYLGWYLQKPGQSPQLLIFRVSNRFSGVPDRFSGSGSGTDFTLKISRVEAEDVGVYYCFQGSHIPRTFGQGTRLEIK

SEQ ID NO: 28 - protein, 111 aa, Artificial Sequence
  zuL5n
DVVMTQSPLSLPVTLGQPASISCRSSQNIVHQQGTYLGWYLQKPGQSPQLLIFRVSNRFSGVPDRFSGSGSGTDFTLKISRVEAEDVGVYYCFQGSHIPRTFGQGTRLEIK

SEQ ID NO: 29 - protein, 112 aa, Artificial Sequence
  zuL5o
DVVMTQSPLSLPVTLGQPASISCRSSQNIVHQNGQTYLGWYLQKPGQSPQLLIFRVSNRFSGVPDRFSGSGSGTDFTLKISRVEAEDVGVYYCFQGSHNPRTFGQGTRLEIK

SEQ ID NO: 30 - protein, 112 aa, Artificial Sequence
  zuL5p
DVVMTQSPLSLPVTLGQPASISCRSSQNIVHQNGQTYLGWYLQKPGQSPQLLIFRVSNRFSGVPDRFSGSGSGTDFTLKISRVEAEDVGVYYCFQGSHHPRTFGQGTRLEIK

SEQ ID NO: 31 - protein, 112 aa, Artificial Sequence
  zuL5q
DVVMTQSPLSLPVTLGQPASISCRSSQNIVHQNGQTYLGWYLQKPGQSPQLLIFRVSNRFLGVPDRFSGSGSGTDFTLKISRVEAEDVGVYYCFQGSHIPRTFGQGTRLEIK

SEQ ID NO: 32 - protein, 25 aa, Artificial Sequence
  H-FR1
  MISC_FEATURE (5)..(5): Xaa = Q or V
  MISC_FEATURE (7)..(7): Xaa = S or P
  MISC_FEATURE (11)..(11): Xaa = V or L
  MISC_FEATURE (12)..(12): Xaa = V or K
  MISC_FEATURE (13)..(13): Xaa = R or K
  MISC_FEATURE (20)..(20): Xaa = L or V
QVQLXQXGAEXXXPGASVKXSCKAS

SEQ ID NO: 33 - protein, 25 aa, Artificial Sequence
  H-FR1-2
  MISC_FEATURE (20)..(20): X = V or L
QVQLVQSGAEVKKPGASVKXSCKAS

SEQ ID NO: 34 - protein, 25 aa, Artificial Sequence
  H-FR1-3
QVQLQQPGAELVRPGASVKLSCKAS

SEQ ID NO: 35 - protein, 25 aa, Artificial Sequence
  H-FR1-4
QVQLVQSGAEVKKPGASVKLSCKAS

SEQ ID NO: 36 - protein, 25 aa, Artificial Sequence [sequence omitted]

SEQ ID NO: 37 - protein, 10 aa, Artificial Sequence
  H-CDR1-1
  MISC_FEATURE (5)..(5): Xaa = T or R
GFSFXTYWIT

SEQ ID NO: 38 - protein, 10 aa, Artificial Sequence
  H-CDR1-2
GFSFTTYWIT

SEQ ID NO: 39 - protein, 10 aa, Artificial Sequence
  H-CDR1-3
GFSFRTYWIT

SEQ ID NO: 40 - protein, 14 aa, Artificial Sequence
  H-FR2-1
  MISC_FEATURE (3)..(3): Xaa = K or R
  MISC_FEATURE (5)..(5): Xaa = R or A
WVXQXPGQGLEWIG

SEQ ID NO: 41 - protein, 14 aa, Artificial Sequence
  H-FR2-2
WVKQRPGQGLEWIG

SEQ ID NO: 42 - protein, 14 aa, Artificial Sequence
  H-FR2-3
WVRQAPGQGLEWIG

SEQ ID NO: 43 - protein, 17 aa, Artificial Sequence
  H-CDR2-1
  MISC_FEATURE (7)..(7): X = A or S
MIHPSDXETRLSQTFKD

SEQ ID NO: 44 - protein, 17 aa, Artificial Sequence
  H-CDR2-2
MIHPSDSETRLSQTFKD

SEQ ID NO: 45 - protein, 17 aa, Artificial Sequence
  H-CDR2-3
MIHPSDAETRLSQTFKD

SEQ ID NO: 46 - protein, 32 aa, Artificial Sequence
  H-FR3-1
  MISC_FEATURE (6)..(6): Xaa = V or A
  MISC_FEATURE (8)..(8): Xaa = K or E
  MISC_FEATURE (10)..(10): Xaa = S or T
  MISC_FEATURE (11)..(11): Xaa = N or S
  MISC_FEATURE (16)..(16): Xaa = E or Q
  MISC_FEATURE (20)..(20): Xaa = P or L
  MISC_FEATURE (21)..(21): Xaa = T or R
  MISC_FEATURE (25)..(25): Xaa = S or T
  MISC_FEATURE (31)..(31): Xaa = V or A
KATLTXDXSXXTAYMXFSSXXSEDXAVYYCXR

SEQ ID NO: 47 - protein, 32 aa, Artificial Sequence
  H-FR3-2
KATLTVDKSSNTAYMQFSSPTSEDSAVYYCVR

SEQ ID NO: 48 - protein, 32 aa, Artificial Sequence
  H-FR3-3
KATLTADKSTNTAYMELSSLRSEDTAVYYCAR

SEQ ID NO: 49 - protein, 32 aa, Artificial Sequence
  H-FR3-4
KATLTADESTNTAYMELSSLRSEDTAVYYCAR

SEQ ID NO: 50 - protein, 32 aa, Artificial Sequence
  H-FR3-5
KATLTADKSTSTAYMELSSLRSEDTAVYYCAR

SEQ ID NO: 51 - protein, 4 aa, Artificial Sequence
  H-CDR3-1
  MISC_FEATURE (1)..(1): Xaa = A or V
DFDV

SEQ ID NO: 52 - protein, 4 aa, Artificial Sequence
  H-CDR3-2
DFDV

SEQ ID NO: 53 - protein, 4 aa, Artificial Sequence
  H-CDR3-3
DFDV

SEQ ID NO: 54 - protein, 11 aa, Artificial Sequence
  H-FR4-1
  MISC_FEATURE (3)..(3): Xaa = A or G
  MISC_FEATURE (6)..(6): Xaa = T or L
WGXGTXVTVSS

SEQ ID NO: 55 - protein, 11 aa, Artificial Sequence
  H-FR4-2
WGAGTTVTVSS

SEQ ID NO: 56 - protein, 11 aa, Artificial Sequence
  H-FR4-3
WGQGTLVTVSS

SEQ ID NO: 57 - protein, 23 aa, Artificial Sequence
  L-FR1-1
  MISC_FEATURE (3)..(3): Xaa = L or V
  MISC_FEATURE (7)..(7): Xaa = T or S
  MISC_FEATURE (14)..(14): Xaa = S or T
  MISC_FEATURE (15)..(15): Xaa = L or P
  MISC_FEATURE (17)..(17): Xaa = D, Q, or E
  MISC_FEATURE (18)..(18): Xaa = Q or P
DVXMTQXPLSLPVXXGXXASISC

SEQ ID NO: 58 - protein, 23 aa, Artificial Sequence
  L-FR1-2
DVLMTQTPLSLPVSLGDQASISC

SEQ ID NO: 59 - protein, 23 aa, Artificial Sequence
  L-FR1-3
DVVMTQTPLSLPVTLGQPASISC

SEQ ID NO: 60 - protein, 23 aa, Artificial Sequence
  L-FR1-4
DVVMTQTPLSLPVTPGEPASISC

SEQ ID NO: 61 - protein, 23 aa, Artificial Sequence
  L-FR1-5
DVVMTQSPLSLPVTLGQPASISC

SEQ ID NO: 62 - protein, 16 aa, Artificial Sequence
  L-CDR1-1
  MISC_FEATURE (9)..(9): Xaa = N or Q
  MISC_FEATURE (10)..(10): Xaa = N or T
  MISC_FEATURE (11)..(11): Xaa = A, R, or G
  MISC_FEATURE (12)..(12): Xaa = N, Q, or G
RSSQNIVHXXXXTYLG

SEQ ID NO: 63 - protein, 16 aa, Artificial Sequence
  L-CDR1-2
  MISC_FEATURE (10)..(10): Xaa = N or T
  MISC_FEATURE (11)..(11): Xaa = A, R, or G
RSSQNIVHQXXQTYLG

SEQ ID NO: 64 - protein, 16 aa, Artificial Sequence
  L-CDR1-3
RSSQNIVHNNGNTYLG

SEQ ID NO: 65 - protein, 16 aa, Artificial Sequence
  L-CDR1-4
RSSQNIVHQNGNTYLG

SEQ ID NO: 66 - protein, 16 aa, Artificial Sequence
  L-CDR1-5
RSSQNIVHNNGQTYLG

SEQ ID NO: 67 - protein, 20 aa, Artificial Sequence
  L-CDR1-6
RSSQNIVHQNAQTYLGWYLQ

SEQ ID NO: 68 - protein, 16 aa, Artificial Sequence
  L-CDR1-7
RSSQNIVHQNGQTYLG

SEQ ID NO: 69 - protein, 16 aa, Artificial Sequence
  L-CDR1-8
RSSQNIVHQTGQTYLG

SEQ ID NO: 70 - protein, 16 aa, Artificial Sequence
  L-CDR1-9
RSSQNIVHQNRQTYLG

SEQ ID NO: 71 - protein, 16 aa, Artificial Sequence
  L-CDR1-10
RSSQNIVHQNAGTYLG

SEQ ID NO: 72 - protein, 14 aa, Artificial Sequence
  L-FR2-1
  MISC_FEATURE (11)..(11): X = K or Q
WYLQKPGQSPXLLI

SEQ ID NO: 73 - protein, 14 aa, Artificial Sequence
  L-FR2-2
WYLQKPGQSPKLLI

SEQ ID NO: 74 - protein, 14 aa, Artificial Sequence
  L-FR2-3
WYLQKPGQSPQLLI

SEQ ID NO: 75 - protein, 8 aa, Artificial Sequence
  L-CDR2-1
  MISC_FEATURE (2)..(2): Xaa = K or R
  MISC_FEATURE (8)..(8): Xaa = S or L
FXVSNRFX

SEQ ID NO: 76 - protein, 8 aa, Artificial Sequence
  L-CDR2-2
FKVSNRFS

SEQ ID NO: 77 - protein, 8 aa, Artificial Sequence
  L-CDR2-3
FRVSNRFS

SEQ ID NO: 78 - protein, 8 aa, Artificial Sequence
  L-CDR2-4
FRVSNRFL

SEQ ID NO: 79 - protein, 32 aa, Artificial Sequence
  L-FR3-1
  MISC_FEATURE (27)..(27): Xaa = L or V
GVPDRFSGSGSGTDFTLKISRVEAEDXGVYYC

SEQ ID NO: 80 - protein, 32 aa, Artificial Sequence
  L-FR3-2
GVPDRFSGSGSGTDFTLKISRVEAEDLGVYYC

SEQ ID NO: 81 - protein, 32 aa, Artificial Sequence
  L-FR3-3
GVPDRFSGSGSGTDFTLKISRVEAEDVGVYYC

SEQ ID NO: 82 - protein, 9 aa, Artificial Sequence
  L-CDR3-1
  MISC_FEATURE (6)..(6): Xaa = I, N, or H
FQGSHXPRT

SEQ ID NO: 83 - protein, 9 aa, Artificial Sequence
  L-CDR3-2
FQGSHIPRT

SEQ ID NO: 84 - protein, 9 aa, Artificial Sequence
  L-CDR3-3
FQGSHNPRT

SEQ ID NO: 85 - protein, 9 aa, Artificial Sequence
  L-CDR3-4
FQGSHHPRT

SEQ ID NO: 86 - protein, 10 aa, Artificial Sequence
  L-FR4-1
  MISC_FEATURE (3)..(3): Xaa = G or Q
FGXGTRLEIK

SEQ ID NO: 87 - protein, 10 aa, Artificial Sequence
  L-FR4-2
FGGGTRLEIK

SEQ ID NO: 88 - protein, 10 aa, Artificial Sequence
  L-FR4-3
FGQGTRLEIK

SEQ ID NO: 89 - protein, 10 aa, Artificial Sequence
  xiH-CDR1
GFSFTTYWIT

SEQ ID NO: 90 - protein, 17 aa, Artificial Sequence
  xiH-CDR2
MIHPSDSETRLSQTFKD

SEQ ID NO: 91 - protein, 4 aa, Artificial Sequence
  xiH-CDR3
DFDV

SEQ ID NO: 92 - protein, 10 aa, Artificial Sequence
  zuH3-CDR1
GFSFTTYWIT

SEQ ID NO: 93 - protein, 17 aa, Artificial Sequence
  zuH3-CDR2
MIHPSDSETRLSQTFKD

SEQ ID NO: 94 - protein, 4 aa, Artificial Sequence
  zuH3-CDR3
DFDV

SEQ ID NO: 95 - protein, 10 aa, Artificial Sequence
  zuH5-CDR1
GFSFTTYWIT

SEQ ID NO: 96 - protein, 17 aa, Artificial Sequence
  zuH5-CDR2
MIHPSDSETRLSQTFKD

SEQ ID NO: 97 - protein, 4 aa, Artificial Sequence
  zuH5-CDR3
DFDV

SEQ ID NO: 98 - protein, 10 aa, Artificial Sequence
  zuH3a-CDR1
GFSFTTYWIT

SEQ ID NO: 99 - protein, 17 aa, Artificial Sequence
  zuH3a-CDR2
MIHPSDAETRLSQTFKD

SEQ ID NO: 100 - protein, 4 aa, Artificial Sequence
  zuH3a-CDR3
DFDV

SEQ ID NO: 101 - protein, 10 aa, Artificial Sequence
  zuH3b-CDR1
GFSFTTYWIT

SEQ ID NO: 102 - protein, 17 aa, Artificial Sequence
  zuH3b-CDR2
MIHPSDAETRLSQTFKD

SEQ ID NO: 103 - protein, 4 aa, Artificial Sequence
  zuH3b-CDR3
DFDV

SEQ ID NO: 104 - protein, 10 aa, Artificial Sequence
  zuH3c-CDR1
GFSFTTYWIT

SEQ ID NO: 105 - protein, 17 aa, Artificial Sequence
  zuH3c-CDR2
MIHPSDAETRLSQTFKD

SEQ ID NO: 106 - protein, 4 aa, Artificial Sequence
  zuH3c-CDR3
DFDV

SEQ ID NO: 107 - protein, 10 aa, Artificial Sequence
  zuH3d-CDR1
GFSFTTYWIT

SEQ ID NO: 108 - protein, 17 aa, Artificial Sequence
  zuH3d-CDR2
MIHPSDAETRLSQTFKD

SEQ ID NO: 109 - protein, 4 aa, Artificial Sequence
  zuH3d-CDR3
DFIV

SEQ ID NO: 110 - protein, 10 aa, Artificial Sequence
  zuH3e-CDR1
GFHFTTYWIT

SEQ ID NO: 111 - protein, 17 aa, Artificial Sequence
  zuH3e-CDR2
MIHPSDAETRLSQTFKD

SEQ ID NO: 112 - protein, 4 aa, Artificial Sequence
  zuH3e-CDR3
DFDV

SEQ ID NO: 113 - protein, 10 aa, Artificial Sequence
  zuH3f-CDR1
GFSFRTYWIT

SEQ ID NO: 114 - protein, 17 aa, Artificial Sequence
  zuH3f-CDR2
MIHPSDAETRLSQTFKD

SEQ ID NO: 115 - protein, 4 aa, Artificial Sequence
  zuH3f-CDR3
DFDV

SEQ ID NO: 116 - protein, 16 aa, Artificial Sequence
  xiL-CDR1
RSSQNIVHNNGNTYLG

SEQ ID NO: 117 - protein, 8 aa, Artificial Sequence
  xiL-CDR2
FKVSNRFS

SEQ ID NO: 118 - protein, 9 aa, Artificial Sequence
  xiL-CDR3
FQGSHIPRT

SEQ ID NO: 119 - protein, 16 aa, Artificial Sequence
  zuL2-CDR1
RSSQNIVHNNGNTYLG

SEQ ID NO: 120 - protein, 8 aa, Artificial Sequence
  zuL2-CDR2
FKVSNRFS

SEQ ID NO: 121 - protein, 9 aa, Artificial Sequence
  zuL2-CDR3
FQGSHIPRT

SEQ ID NO: 122 - protein, 16 aa, Artificial Sequence
  zuL3-CDR1
RSSQNIVHNNGNTYLG

SEQ ID NO: 123 - protein, 8 aa, Artificial Sequence
  zuL3-CDR2
FKVSNRFS

SEQ ID NO: 124 - protein, 9 aa, Artificial Sequence
  zuL3-CDR3
FQGSHIPRT

SEQ ID NO: 125 - protein, 16 aa, Artificial Sequence
  zuL4-CDR1
RSSQNIVHNNGNTYLG

SEQ ID NO: 126 - protein, 8 aa, Artificial Sequence
  zuL4-CDR2
FKVSNRFS

SEQ ID NO: 127 - protein, 9 aa, Artificial Sequence
  zuL4-CDR3
FQGSHIPRT

SEQ ID NO: 128 - protein, 16 aa, Artificial Sequence
  zuL5-CDR1
RSSQNIVHNNGNTYLG

SEQ ID NO: 129 - protein, 8 aa, Artificial Sequence
  zuL5-CDR2
FKVSNRFS

SEQ ID NO: 130 - protein, 9 aa, Artificial Sequence
  zuL5-CDR3
FQGSHIPRT

SEQ ID NO: 131 - protein, 16 aa, Artificial Sequence
  zuL5a-CDR1
RSSQNIVHQNGNTYLG

SEQ ID NO: 132 - protein, 8 aa, Artificial Sequence
  zuL5a-CDR2
FKVSNRFS

SEQ ID NO: 133 - protein, 9 aa, Artificial Sequence
  zuL5a-CDR3
FQGSHIPRT

SEQ ID NO: 134 - protein, 16 aa, Artificial Sequence
  zuL5b-CDR1
RSSQNIVHNQGNTYLG

SEQ ID NO: 135 - protein, 8 aa, Artificial Sequence
  zuL5b-CDR2
FKVSNRFS

SEQ ID NO: 136 - protein, 9 aa, Artificial Sequence
  zuL5b-CDR3
FQGSHIPRT

SEQ ID NO: 137 - protein, 16 aa, Artificial Sequence
  zuL5c-CDR1
RSSQNIVHNNGQTYLG

SEQ ID NO: 138 - protein, 8 aa, Artificial Sequence
  zuL5c-CDR2
FKVSNRFS

SEQ ID NO: 139 - protein, 9 aa, Artificial Sequence
  zuL5c-CDR3
FQGSHIPRT

SEQ ID NO: 140 - protein, 16 aa, Artificial Sequence
  zuL5d-CDR1
RSSQNIVHNNGNTYLG

SEQ ID NO: 141 - protein, 8 aa, Artificial Sequence
  zuL5d-CDR2
FRVSNRFS

SEQ ID NO: 142 - protein, 9 aa, Artificial Sequence
  zuL5d-CDR3
FQGSHIPRT

SEQ ID NO: 143 - protein, 16 aa, Artificial Sequence
  zuL5e-CDR1
RSSQNIVHQNAQTYLG

SEQ ID NO: 144 - protein, 8 aa, Artificial Sequence
  zuL5e-CDR2
FKVSNRFS

SEQ ID NO: 145 - protein, 9 aa, Artificial Sequence
  zuL5e-CDR3
FQGSHIPRT

SEQ ID NO: 146 - protein, 16 aa, Artificial Sequence
  zuL5f-CDR1
RSSQNIVHQNAQTYLG

SEQ ID NO: 147 - protein, 8 aa, Artificial Sequence
  zuL5f-CDR2
FRVSNRFS

SEQ ID NO: 148 - protein, 9 aa, Artificial Sequence
  zuL5f-CDR3
FQGSHIPRT

SEQ ID NO: 149 - protein, 16 aa, Artificial Sequence
  zuL5g-CDR1
RSSQNIVHQNGQTYLG

SEQ ID NO: 150 - protein, 8 aa, Artificial Sequence
  zuL5g-CDR2
FRVSNRFS

SEQ ID NO: 151 - protein, 9 aa, Artificial Sequence
  zuL5g-CDR3
FQGSHIPRT

SEQ ID NO: 152 - protein, 16 aa, Artificial Sequence
  zuL5h-CDR1
RSSQNIVHQHGQTYLG

SEQ ID NO: 153 - protein, 8 aa, Artificial Sequence
  zuL5h-CDR2
FRVSNRFS

SEQ ID NO: 154 - protein, 9 aa, Artificial Sequence
  zuL5h-CDR3
FQGSHIPRT

SEQ ID NO: 155 - protein, 16 aa, Artificial Sequence
  zuL5i-CDR1
RSSQNIVHQTGQTYLG

SEQ ID NO: 156 - protein, 8 aa, Artificial Sequence
  zuL5i-CDR2
FRVSNRFS

SEQ ID NO: 157 - protein, 9 aa, Artificial Sequence
  zuL5i-CDR3
FQGSHIPRT

SEQ ID NO: 158 - protein, 16 aa, Artificial Sequence
  zuL5j-CDR1
RSSQNIVHQNRQTYLG

SEQ ID NO: 159 - protein, 8 aa, Artificial Sequence
  zuL5j-CDR2
FRVSNRFS

SEQ ID NO: 160 - protein, 9 aa, Artificial Sequence
  zuL5j-CDR3
FQGSHIPRT

SEQ ID NO: 161 - protein, 16 aa, Artificial Sequence
  zuL5k-CDR1
RSSQNIVHQNAGTYLG

SEQ ID NO: 162 - protein, 8 aa, Artificial Sequence
  zuL5k-CDR2
FRVSNRFS

SEQ ID NO: 163 - protein, 9 aa, Artificial Sequence
  zuL5k-CDR3
FQGSHIPRT

SEQ ID NO: 164 - protein, 15 aa, Artificial Sequence
  zuL5L-CDR1
RSSQNIVHQNQTYLG

SEQ ID NO: 165 - protein, 8 aa, Artificial Sequence
  zuL5L-CDR2
FRVSNRFS

SEQ ID NO: 166 - protein, 9 aa, Artificial Sequence
  zuL5L-CDR3
FQGSHIPRT

SEQ ID NO: 167 - protein, 15 aa, Artificial Sequence
  zuL5m-CDR1
RSSQNIVHQGQTYLG

SEQ ID NO: 168 - protein, 8 aa, Artificial Sequence
  zuL5m-CDR2
FRVSNRFS

SEQ ID NO: 169 - protein, 9 aa, Artificial Sequence
  zuL5m-CDR3
FQGSHIPRT

SEQ ID NO: 170 - protein, 15 aa, Artificial Sequence
  zuL5n-CDR1
RSSQNIVHQQGTYLG

SEQ ID NO: 171 - protein, 8 aa, Artificial Sequence
  zuL5n-CDR2
FRVSNRFS

SEQ ID NO: 172 - protein, 9 aa, Artificial Sequence
  zuL5n-CDR3
FQGSHIPRT

SEQ ID NO: 173 - protein, 16 aa, Artificial Sequence
  zuL5o-CDR1
RSSQNIVHQNGQTYLG

SEQ ID NO: 174 - protein, 8 aa, Artificial Sequence
  zuL5o-CDR2
FRVSNRFS

SEQ ID NO: 175 - protein, 9 aa, Artificial Sequence
  zuL5o-CDR3
FQGSHNPRT

SEQ ID NO: 176 - protein, 16 aa, Artificial Sequence
  zuL5p-CDR1
RSSQNIVHQNGQTYLG

SEQ ID NO: 177 - protein, 8 aa, Artificial Sequence
  zuL5p-CDR2
FRVSNRFS

SEQ ID NO: 178 - protein, 9 aa, Artificial Sequence
  zuL5p-CDR3
FQGSHHPRT

SEQ ID NO: 179 - protein, 16 aa, Artificial Sequence
  zuL5q-CDR1
RSSQNIVHQNGQTYLG

SEQ ID NO: 180 - protein, 8 aa, Artificial Sequence
  zuL5q-CDR2
FRVSNRFL

SEQ ID NO: 181 - protein, 9 aa, Artificial Sequence
  zuL5q-CDR3
FQGSHIPRT

SEQ ID NO: 182 - protein, 11 aa, Artificial Sequence
  HEG1 fragment
SKSPSLVSLPT

SEQ ID NO: 183 - protein, 111 aa, Artificial Sequence
  SLURPgpi
GSLKCYTCKEPMTSASCRTITRCKPEDTACMTTLVTVEAEYPFNQSPVVTRSCSSSCVATDPDSIGAAHLIFCCFRDLCNSELSRDGAPSLGSPGGLLLALALFLLLGVLL

SEQ ID NO: 184 - protein, 34 aa, Artificial Sequence
  Signal sequence
MASPRASRWPPPLLLLLLPLLLLPPAAPGTRDGT

SEQ ID NO: 185 - protein, 13 aa, Artificial Sequence
  IgG-binding protein 1
  MISC_FEATURE (6)..(6): Xaa = R, C, D, E, 2-amino suberic acid, or diaminopropionic acid
DCAYHXGELVWCT

SEQ ID NO: 186 - protein, 17 aa, Artificial Sequence
  IgG-binding protein 2
  MISC_FEATURE (8)..(8): Xaa = R, C, D, E, 2-amino suberic acid, or diaminopropionic acid
GPDCAYHXGELVWCTFH

SEQ ID NO: 187 - protein, 13 aa, Artificial Sequence
  IgG-binding protein 3
  MISC_FEATURE (6)..(6): Xaa = R, C, D, E, 2-amino suberic acid, or diaminopropionic acid
RCAYHXGELVWCS

SEQ ID NO: 188 - protein, 17 aa, Artificial Sequence
  IgG-binding protein 4
  MISC_FEATURE (8)..(8): Xaa = R, C, D, E, 2-amino suberic acid, or diaminopropionic acid
GPRCAYHXGELVWCSFH

SEQ ID NO: 189 - protein, 17 aa, Artificial Sequence
  IgG-binding protein 5
  MISC_FEATURE (8)..(8): Xaa = R, C, D, E, 2-amino suberic acid, or diaminopropionic acid
SPDCAYHXGELVWCTFH

SEQ ID NO: 190 - protein, 17 aa, Artificial Sequence
  IgG-binding protein 6
  MISC_FEATURE (8)..(8): Xaa = R, C, D, E, 2-amino suberic acid, or diaminopropionic acid
GDDCAYHXGELVWCTFH

SEQ ID NO: 191 - protein, 17 aa, Artificial Sequence
  IgG-binding protein 7
  MISC_FEATURE (8)..(8): Xaa = R, C, D, E, 2-amino suberic acid, or diaminopropionic acid
GPSCAYHXGELVWCTFH

SEQ ID NO: 192 - protein, 17 aa, Artificial Sequence
  IgG-binding protein 8
  MISC_FEATURE (8)..(8): Xaa = R, C, D, E, 2-amino suberic acid, or diaminopropionic acid
GPDCAYHXGELVWCSFH

SEQ ID NO: 193 - protein, 17 aa, Artificial Sequence
  IgG-binding protein 9
  MISC_FEATURE (8)..(8): Xaa = R, C, D, E, 2-amino suberic acid, or diaminopropionic acid
GPDCAYHXGELVWCTHH

SEQ ID NO: 194 - protein, 17 aa, Artificial Sequence
  IgG-binding protein 10
  MISC_FEATURE (8)..(8): Xaa = R, C, D, E, 2-amino suberic acid, or diaminopropionic acid
GPDCAYHXGELVWCTFY

SEQ ID NO: 195 - protein, 17 aa, Artificial Sequence
  IgG-binding protein 11
  MISC_FEATURE (8)..(8): Xaa = R, C, D, E, 2-amino suberic acid, or diaminopropionic acid
SPDCAYHXGELVWCTFY

SEQ ID NO: 196 - protein, 17 aa, Artificial Sequence
  IgG-binding protein 12
  MISC_FEATURE (8)..(8): Xaa = R, C, D, E, 2-amino suberic acid, or diaminopropionic acid
SDDCAYHXGELVWCTFY

SEQ ID NO: 197 - protein, 17 aa, Artificial Sequence
  IgG-binding protein 13
  MISC_FEATURE (8)..(8): Xaa = R, C, D, E, 2-amino suberic acid, or diaminopropionic acid
RGNCAYHXGQLVWCTYH

SEQ ID NO: 198 - protein, 17 aa, Artificial Sequence
  IgG-binding protein 14
  MISC_FEATURE (2)..(2): Xaa = homocystein
  MISC_FEATURE (8)..(8): Xaa = R, C, D, E, 2-amino suberic acid, or diaminopropionic acid
  MISC_FEATURE (16)..(16): Xaa = homocystein
GXDCAYHXGELVWCTXH

SEQ ID NO: 199 - protein, 13 aa, Artificial Sequence
  IgG-binding protein 15
  MISC_FEATURE (6)..(6): Xaa = R, C, D, E, 2-amino suberic acid, or diaminopropionic acid
DCTYHXGNLVWCT

SEQ ID NO: 200 - protein, 13 aa, Artificial Sequence
  IgG-binding protein 16
  MISC_FEATURE (6)..(6): Xaa = R, C, D, E, 2-amino suberic acid, or diaminopropionic acid
DCAYHXGNLVWCT

SEQ ID NO: 201 - protein, 13 aa, Artificial Sequence
  IgG-binding protein 17
  MISC_FEATURE (6)..(6): Xaa = R, C, D, E, 2-amino suberic acid, or diaminopropionic acid
DCTYHXGELVWCT

SEQ ID NO: 202 - protein, 13 aa, Artificial Sequence
  IgG-binding protein 18
  MISC_FEATURE (6)..(6): Xaa = R, C, D, E, 2-amino suberic acid, or diaminopropionic acid
DCAWHXGELVWCT

SEQ ID NO: 203 - protein, 13 aa, Artificial Sequence
  IgG-binding protein 19
  MISC_FEATURE (6)..(6): Xaa = R, C, D, E, 2-amino suberic acid, or diaminopropionic acid
DCTYTXGNLVWCT

SEQ ID NO: 204 - protein, 13 aa, Artificial Sequence
  IgG-binding protein 20
  MISC_FEATURE (6)..(6): Xaa = R, C, D, E, 2-amino suberic acid, or diaminopropionic acid
DCAYTXGNLVWCT

SEQ ID NO: 205 - protein, 13 aa, Artificial Sequence
  IgG-binding protein 21
  MISC_FEATURE (6)..(6): Xaa = R, C, D, E, 2-amino suberic acid, or diaminopropionic acid
DCSYTXGNLVWCT

SEQ ID NO: 206 - protein, 13 aa, Artificial Sequence
  IgG-binding protein 22
  MISC_FEATURE (6)..(6): Xaa = R, C, D, E, 2-amino suberic acid, or diaminopropionic acid
DCTWTXGNLVWCT

SEQ ID NO: 207 - protein, 13 aa, Artificial Sequence
  IgG-binding protein 23
  MISC_FEATURE (6)..(6): Xaa = R, C, D, E, 2-amino suberic acid, or diaminopropionic acid
DCTYHXGNLVWCT

SEQ ID NO: 208 - protein, 13 aa, Artificial Sequence
  IgG-binding protein 24
  MISC_FEATURE (6)..(6): Xaa = R, C, D, E, 2-amino suberic acid, or diaminopropionic acid
DCTYRXGNLVWCT

SEQ ID NO: 209 - protein, 13 aa, Artificial Sequence
  IgG-binding protein 25
  MISC_FEATURE (6)..(6): Xaa = R, C, D, E, 2-amino suberic acid, or diaminopropionic acid
DCTYSXGNLVWCT

SEQ ID NO: 210 - protein, 13 aa, Artificial Sequence
  IgG-binding protein 26
  MISC_FEATURE (6)..(6): Xaa = R, C, D, E, 2-amino suberic acid, or diaminopropionic acid
DCTYTXGNLVWCT

SEQ ID NO: 211 - protein, 13 aa, Artificial Sequence
  IgG-binding protein 27
  MISC_FEATURE (6)..(6): Xaa = R, C, D, E, 2-amino suberic acid, or diaminopropionic acid
DCTYTXGELVWCT

SEQ ID NO: 212 - protein, 13 aa, Artificial Sequence
  IgG-binding protein 28
  MISC_FEATURE (6)..(6): Xaa = R, C, D, E, 2-amino suberic acid, or diaminopropionic acid
DCTYTXGRLVWCT

SEQ ID NO: 213 - protein, 13 aa, Artificial Sequence
  IgG-binding protein 29
  MISC_FEATURE (6)..(6): Xaa = R, C, D, E, 2-amino suberic acid, or diaminopropionic acid
DCTYTXGDLVWCT

SEQ ID NO: 214 - protein, 13 aa, Artificial Sequence
  IgG-binding protein 30
  MISC_FEATURE (6)..(6): Xaa = R, C, D, E, 2-amino suberic acid, or diaminopropionic acid
DCTYTXGNLIWCT

SEQ ID NO: 215 - protein, 110 aa, Artificial Sequence
  IgG1 CH2
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 216 - protein, 11 aa, Artificial Sequence
  IgG1 hinge region
CPPCPAPELLG

SEQ ID NO: 217 - protein, 11 aa, Artificial Sequence
  IgG4 hinge region
CPSCPAPEFLG

SEQ ID NO: 218 - protein, 11 aa, Artificial Sequence
  IgG4PE hinge region
CPPCPAPEFEG

SEQ ID NO: 219 - protein, 104 aa, Artificial Sequence
  IgG1 CH2 region
GPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 220 - protein, 104 aa, Artificial Sequence
  IgG4 or IgG4PE CH2 region
GPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAK

SEQ ID NO: 221 - protein, 19 aa, Artificial Sequence
  Heavy chain signal sequence VH1
MDWTWRILFLVAAATGAHS

SEQ ID NO: 222 - protein, 19 aa, Artificial Sequence
  Heavy chain signal sequence VH2
MDILCSTLLLLTVPSWVLS

SEQ ID NO: 223 - protein, 19 aa, Artificial Sequence
  Heavy chain signal sequence VH3
MEFGLSWVFLVALLRGVQC

SEQ ID NO: 224 - protein, 19 aa, Artificial Sequence
  Heavy chain signal sequence VH4
MKHLWFFLLLVAAPRWVLS

SEQ ID NO: 225 - protein, 19 aa, Artificial Sequence
  Heavy chain signal sequence VH5
MGSTAILALLLAVLQGVCA

SEQ ID NO: 226 - protein, 20 aa, Artificial Sequence
  Heavy chain signal sequence VH6
MSVSFLIFLPVLGLPWGVLS

SEQ ID NO: 227 - protein, 22 aa, Artificial Sequence
  Light chain signal sequence KL1
MDMRVPAQLLGLLLLWVPGARC

SEQ ID NO: 228 - protein, 20 aa, Artificial Sequence
  Light chain signal sequence KL2
MRLPAQLLGLLMLWVPGSSG

SEQ ID NO: 229 - protein, 19 aa, Artificial Sequence
  Mouse heavy chain signal sequence
MGWSSIILFLVATTTGVHS

SEQ ID NO: 230 - protein, 19 aa, Artificial Sequence
  Mouse light chain signal sequence
MKLPVRLLVLMFWIPASIS